SEQUENCE LISTING (114 sequences)

SEQ ID NO: 1 - protein, 288 aa, Streptomyces albireticuli
MPQESAQELKVTADEVGDWYDRFGDIYHETLGESVHCGLWFPPDEPHPTSMDLVDLSSRAQDRYTDYLIETLDPRPGDHVLDIGCGTGRSALRLVQQRDARVTGVAISKEQIARADRLANEHGLTDRLTFAYADAQALPYEDGTFDRAWAVESICHMDRAKALQEAWRVLRPGGDLMVLESVLTGELTAEDTAVFQVMLASNLPPTLPEFFGLVGDAGFETLELKDLSANLAMTMNVMALVCHDRKEEFTERFGAEFMEGVVQGLPKAREVVARKTRFFLVMLRKPLA

SEQ ID NO: 2 - protein, 291 aa, Streptomyces clavuligerus
MPQELAGELRVTAAQVGAWYDQFGDIYHQTLGESIHCGLWFPPDEPHPARVDLVSLSSEAQDRFTDYLIKTLDPHADQHVLDIGCGTGRSALRLSQQRGAKVTGVAISKVQIEHANRLAETHDLSDRLVFEHADAMHLPYEDESFDSAWAIESLCHMDRAKALREAYRVLRPGGDFLLLESVLTNPLTEAEATSLDTMLAANTPLWLPEFFELITRAGFETLELKDLSANLAMTMNVLELVCHDRREEFTRRFGAEFTELLMAGLPEARNITARKTRFFMLLLRKPPVPAN

SEQ ID NO: 3 - protein, 289 aa, Streptomyces blastmyceticus
MPQEARTPQQQVTADEVGDWYDKFGEVYHLTLGESVHCGLWFPPDAPVPQDMELVTMSSQAQDRYTDYLIETLDPKAGQHLLDIGCGTGRTALKAARQRGIAVTGVAVSKEQIAAANRLAAGHGLTERLTFEVADAMRLPYEDESFDCAWAIESLCHMDRAKALGEAWRVLKPGGDLLVLESVVTEELTEPETALFETLYAANVPPRLGEFFDIVSGAGFHTLSLKDLSANLAMTMNVFALGVYSRRAEFTERFGAEFVDGLLAGLGSAQETLIRKTRFFMATLRKPAV

SEQ ID NO: 4 - protein, 289 aa, Artificial sequence
  Modified from pMT1
MPQEARTPQQQVTADEVGDWYDKFGEVYHLTLGESVHCGLWFPPDAPVPQDMELVTMSSQAQDRYTDYLIETLDPKAGQHLLDIGCGTGRTALKAARQRGIAVTGVAVSKEQIAAANRLAAGHGLTERLTFEVADAMRLPYEDESFDCAWAIESLCHMDRAKALGEAWRVLKPGGDLLVLESVVTEELTEPETALFETLFAANVPPRLGEFFDIVSGAGFHTLSLKDLSANLAMTMNVFALGVYSRRAEFTERFGAEFVDGLLAGLGSAQETLIRKTRFFMATLRKPAV

SEQ ID NO: 5 - protein, 289 aa, Artificial sequence
  Modified from pMT1
MPQEARTPQQQVTADEVGDWYDKFGEVYHLTLGESVHCGLWFPPDAPVPQDMELVTMSSQAQDRYTDYLIETLDPKAGQHLLDIGCGTGRTALKAARQRGIAVTGVAVSKEQIAAANRLAAGHGLTERLTFEVADAMRLPYEDESFDCAWAIESLCHMDRAKALGEAWRVLKPGGDLLVLEEVVTEELTEPETALFETLFAANVPPRLGEFFDIVSGAGFHTLSLKDLSANLAMTMNVFALGVYSRRAEFTERFGAEFVDGLLAGLGSAQETLIRKTRFFMATLRKPAV

SEQ ID NO: 6 - protein, 289 aa, Artificial sequence
  Modified from pMT1
MPQEARTPQQQVTADEVGDWYDKFGEVYHLTLGESVHCGLWFPPDAPVPQDMELVTMSSQAQDRYTDYLIETLDPKAGQHLLDIGCGTGRTALKAARQRGIAVTGVAVSKEQIAAANRLAAGHGLTERLTFEVADAMRLPYEDESFDCAWAIESLCHMDRAKALGEAWRVLKPGGDLLVLEEVVTEELTEPETALFETLFAANVPPRLGEFFDIVSGAGFHTLSLKDLSANLAMTMNVFALGVYSRRAEFTERFGAEFVDGLLAGLGSAQETVIRKTRFFMATLRKPAV

SEQ ID NO: 7 - protein, 289 aa, Artificial sequence
  Modified from pMT1
MPQEARTPQQQVTADEVGDWYDKFGEVYHLTLGESVHCGLWFPPDAPVPQDMELVTMSSQAQDRYTDYLIETLDPKAGQHLLDIGCGTGRTALKAARQRGIAVTGVAVSKEQIAAANRLAAGHGLTERLTFEVADAMRLPYEDESFDCAWAIESLCHMDRAKALGEAWRVLKPGGDLLVAEEVVTEELTEPETALFETLFAANVPPRLGEFFDIVSGAGFHTLSLKDLSANLAMTMNVFALGVYSRRAEFTERFGAEFVDGLLAGLGSAQETVIRKTRFFMATLRKPAV

SEQ ID NO: 8 - protein, 289 aa, Artificial sequence
  Modified from pMT1
MPQEARTPQQQVTADEVGDWYDKFGEVYHLTLGESVHCGLWFPPDAPVPQDMELVTMSSQAQDRYTDYLIETLDPKAGQHLLDIGCGTGRTALKAARQRGIAVTGVAVSKEQIAAANRLAAGHGLTERLTFEVADAMRLPYEDESFDCAWAIESLCHMDRAKALGEAWRVLKPGGDLLVAEEVVTEELTEPETALFETLFALNVPPRLGEFFDIVSGAGFHTLSLKDLSANLAMTMNVFALGVYSRRAEFTERFGAEFVDGLLAGLGSAQETVIRKTRFFMATLRKPAV

SEQ ID NO: 9 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRTALKAARQRGIAVTGVAVSKEQIAAANRLAAGHGLTERLTFEVADAMRLPYEDESFDCAWAIESLCHMDRAKALGEAWRVLKPGGDLLVAEEVVTEELTEPETALFETLFALNVPPRLGEFFDIVSGAGFHTLSLKDLSANLAMTMNVFALGVYSRRAEFTERFGAEFVDGLLAGLGSAQETVIRKTRFFMATLRKPAV

SEQ ID NO: 10 - protein, 289 aa, Artificial sequence
  Modified from pMT1
MPQEARTPQQQVTADEVGDWYDKFGEVYHLTLGESVHCGLWFPPDAPVPQDMELVTMSSQAQDRYTDYLIETLDPKAGQHLLDIGCGTGRTALKAARQRGIAVTGVAVSKEQIAAANRLAAGHGLTERLTFEVADAMRLPYEDESFDCAWAIESLPHMDRAKALGEAWRVLKPGGDLLVAEEVVTEELTEPETALFETLFALNVPPRLGEFFDIVSGAGFHTLSLKDLSANLAMTMNVFALGVYSRRAEFTERFGAEFVDGLLAGLGSAQETVIRKTRFFMATLRKPAV

SEQ ID NO: 11 - protein, 289 aa, Artificial sequence
  Modified from pMT1
MPQEARTPQQQVTADEVGDWYDKFGEVYHLTLGESVHCGLWFPPDAPVPQDMELVTMSSQAQDRYTDYLIETLDPKAGQHLLDIGCGTGRTALKAARQRGIAVTGVAVSKEQIAAANRLAAGHGLTERLTFEVADAMRLPYEDESFDCAWAIESLAHMDRAKALGEAWRVLKPGGDLLVAEEVVTEELTEPETALFETLFALNVPPRLGEFFDIVSGAGFHTLSLKDLSANLAMTMNVFALGVYSRRAEFTERFGAEFVDGLLAGLGSAQETVIRKTRFFMATLRKPAV

SEQ ID NO: 12 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLAHMDRAKALGEAWRVLKPGGDLLVAEEVVTEELTEPETALFETLFALNVPPRLGEFFDIVSGAGFHTLSLKDLSANLAMTMNVFALGVYSRRAEFTERFGAEFVDGLLAGLGSAQETVIRKTRFFMATLRKPAV

SEQ ID NO: 13 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSANLAMTMNVFALGVYSRRAEFTERFGAEFVDGLLAGLGSAQETVIRKTRFFMATLRKPAV

SEQ ID NO: 14 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 15 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVAARRAEFTERFGAEFVDGLLAGLPSAQETVIRKTRFFMATLRKPAV

SEQ ID NO: 16 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGDLLVAEEVVTEELTEPETALFRTLFALNVPPRLGEFFDIVSGAGFHTLSLKDLSADLAMTMNVFALGVAARRAEFTERFGAEFVDGLLAGLPSAQETVIRKTRFFMATLRKPAV

SEQ ID NO: 17 - DNA, 885 bp, Streptomyces albireticuli
atgcatcatcatcaccatcacccgcaggagtctgcgcaggaactgaaagttaccgcggacgaagttggggattggtacgatcgtttcggtgacatctaccacgaaaccctcggagaaagcgtgcactgcggcctgtggtttccgccggatgaaccacatccgactagcatggatctggtagacctgtcttctcgcgcccaagatcgttacaccgattacctgatcgaaaccctggatccgcgcccgggcgaccacgtgctggacattggttgtggcaccggtcgttctgcgctccgtctggtacaacaacgcgacgcgcgtgttaccggtgtagctatttctaaagagcagatcgcccgtgcggaccgcctggcgaatgaacacggtctgaccgaccgcctgaccttcgcatacgcagatgctcaagctctgccgtacgaagatggtacctttgatcgcgcttgggcggttgagtccatctgccacatggaccgtgcgaaagctctgcaggaagcgtggcgtgttttacgccctggtggagatctgatggttctggaatctgtgctgaccggtgaactgaccgcggaagatacggctgtgttccaggtaatgctggcgtctaacctgccgccgactctgccggaattcttcggcctggttggtgacgcaggctttgaaaccctggaactgaaagacttatctgcgaacctggctatgaccatgaacgtgatggccctggtttgccatgaccgcaaagaagaattcaccgaacgttttggcgcggaattcatggagggtgtagtacagggtctcccgaaggcgcgcgaagttgttgcacgtaaaacccgttttttcctcgtgatgctgcgcaaaccgctggcttaa

SEQ ID NO: 18 - DNA, 894 bp, Streptomyces clavuligerus
atgcatcatcatcaccatcacccacaggaactggcgggtgaactgcgtgtgaccgctgcccaggtaggcgcgtggtatgatcagttcggtgacatctaccaccaaaccttaggtgagtccatccattgcggtctgtggttcccaccggacgaaccgcatccagctcgtgttgatctggtttctctgtcgagcgaagcgcaggatcgtttcaccgactaccttattaaaacgctggacccgcacgcagaccagcacgttctggatatcggttgtggcactggccgctctgcactgcgtctgtcgcaacagcgtggtgcaaaggttaccggcgtagcaatttccaaagtgcagatcgaacatgccaatcgcctggcggaaacccacgacttgagcgatcgtctggtcttcgaacacgcagatgctatgcatctgccgtatgaagatgaatctttcgattccgcttgggctatcgaatccctctgccacatggaccgcgcgaaagcgctgcgcgaagcttaccgtgtactgcgcccgggtggcgatttcctgctgctggagagcgttctgaccaacccgctgaccgaagcagaagccaccagcttagatactatgctggcggcaaataccccgctgtggctgccagaattctttgaactgatcacccgtgctggttttgaaactctggaactgaaagacctgagcgcgaacctggccatgaccatgaacgttctggaactggtttgccatgatcgtcgcgaagaatttacccgtcgttttggtgctgagtttaccgaactgttgatggctggtctgccagaggcgcgtaacatcaccgcgcgcaagacgcgtttctttatgctgctgctgcgtaagccgccggtgccggcgaactaa

SEQ ID NO: 19 - DNA, 891 bp, Streptomyces blastmyceticus
atgcatcatcatcaccatcacgtcccgcaggaagcccgtaccccgcagcagcaggttaccgccgatgaagtcggcgattggtacgataaattcggcgaagtgtaccatctgactctgggtgaaagcgtgcattgcggtctgtggttcccgccggacgccccggttccgcaggacatggagctggttaccatgtcctctcaggcgcaggatcgttacacggattatctgattgaaaccctggatccgaaagcgggtcagcatctgttagacatcggctgtggtaccggtcgcaccgctctgaaagccgcacgccagcgcggtatcgcggtgaccggtgtagcagttagtaaagaacagattgctgcagcgaaccgcctggcggcaggtcacggtctgactgagcgtctgaccttcgaagtagccgacgctatgcgtctgccgtacgaagacgaatcgttcgactgtgcttgggcgatcgagtcactgtgccacatggatcgtgcaaaggctcttggtgaagcttggcgtgtcttgaaaccgggtggtgacctgctggtactggaatccgtcgtaactgaagaactgactgaaccggaaaccgcactgttcgaaacgctgtacgccgcgaatgttccgccgcgtctgggtgaattctttgatatcgtatctggtgcgggtttccacaccctgagcttaaaagacctgtccgcaaacctggccatgactatgaacgttttcgcactgggtgtgtattctcgtcgtgccgaatttaccgaacgcttcggcgcggaattcgttgacggcctgctggccggtctgggctcggcgcaggaaaccctcattcgcaaaacccgtttctttatggctactctgcgcaagccggcggtctaa

SEQ ID NO: 20 - protein, 289 aa, Artificial sequence
  Modified from pMT1
MPQEARTPQQQVTADEVGDWYDKFGEVYHLTLGESVHCGLWFPPDAPVPQDMELVTMSSVAQDRFTDYLIETLDPKAGQHLLDIGCGTGRTALKAARQRGIAVTGVAVSKEQIAAANRLAAGHGLTERLTFEVADAMRLPYEDESFDCAWAIESLCHMDRAKALGEAWRVLKPGGDLLVAEEVVTEELTEPETALFETLFALNVPPRLGEFFDIVSGAGFHTLSLKDLSANLAMTMNVFALGVYSRRAEFTERFGAEFVDGLLAGLGSAQETVIRKTRFFMATLRKPAV

SEQ ID NO: 21 - protein, 289 aa, Artificial sequence
  Modified from pMT1
MPQEARTPQQQVTADEVGDWYDKFGEVYHLTLGESVHCGLWFPPDAPVPQDMELVTMSSKAQDRFTDYLIETLDPKAGQHLLDIGCGTGRTALKAARQRGIAVTGVAVSKEQIAAANRLAAGHGLTERLTFEVADAMRLPYEDESFDCAWAIESLCHMDRAKALGEAWRVLKPGGDLLVAEEVVTEELTEPETALFETLFALNVPPRLGEFFDIVSGAGFHTLSLKDLSANLAMTMNVFALGVYSRRAEFTERFGAEFVDGLLAGLGSAQETVIRKTRFFMATLRKPAV

SEQ ID NO: 22 - protein, 289 aa, Artificial sequence
  Modified from pMT1
MPQEARTPQQQVTADEVGDWYDKFGEVYHLTLGESVHCGLWFPPDAPVPQDMELVTMSSRAQDRFTDYLIETLDPKAGQHLLDIGCGTGRTALKAARQRGIAVTGVAVSKEQIAAANRLAAGHGLTERLTFEVADAMRLPYEDESFDCAWAIESLCHMDRAKALGEAWRVLKPGGDLLVAEEVVTEELTEPETALFETLFALNVPPRLGEFFDIVSGAGFHTLSLKDLSANLAMTMNVFALGVYSRRAEFTERFGAEFVDGLLAGLGSAQETVIRKTRFFMATLRKPAV

SEQ ID NO: 23 - protein, 289 aa, Artificial sequence
  Modified from pMT1
MPQEARTPQQHVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 24 - protein, 289 aa, Artificial sequence
  Modified from pMT1
MPQEARTPQQQRTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 25 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKCARQRGIAVTGVAVSKEQIAAANRLAAGHGLTERLTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 26 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGQGLTERLTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 27 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTTRLTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 28 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADATRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 29 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGVLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 30 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGYLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 31 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGDLLVAEEVVTEELTESETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 32 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGDLLVAEEVVTEELTEVETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 33 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGDLLVAEEVVTEELTEPETALFETLFALNVPPRLGEFLDIVSGAGFHTLSLKDLSADLAMTMNVFALGVYSRRAEFTERFGAEFVDGLLAGLGSAQETVIRKTRFFMATLRKPAV

SEQ ID NO: 34 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGHAQETVIRKTRFFMATLRKPAV

SEQ ID NO: 35 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSAQEAVIRKTRFFMATLRKPAV

SEQ ID NO: 36 - protein, 289 aa, Artificial sequence
  Modified from pMT1
MPQEARTPQQLVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 37 - protein, 289 aa, Artificial sequence
  Modified from pMT1
MPQEARTPQQPVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 38 - protein, 289 aa, Artificial sequence
  Modified from pMT1
MPQEARTPQQMVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 39 - protein, 289 aa, Artificial sequence
  Modified from pMT1
MPQEARTPQQVVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 40 - protein, 289 aa, Artificial sequence
  Modified from pMT1
MPQEARTPQQWVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 41 - protein, 289 aa, Artificial sequence
  Modified from pMT1
MPQEARTPQQQKTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 42 - protein, 289 aa, Artificial sequence
  Modified from pMT1
MPQEARTPQQQATADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 43 - protein, 289 aa, Artificial sequence
  Modified from pMT1
MPQEARTPQQQGTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 44 - protein, 289 aa, Artificial sequence
  Modified from pMT1
MPQEARTPQQQVLADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 45 - protein, 289 aa, Artificial sequence
  Modified from pMT1
MPQEARTPQQQVMADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 46 - protein, 289 aa, Artificial sequence
  Modified from pMT1
MPQEARTPQQQVQADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 47 - protein, 289 aa, Artificial sequence
  Modified from pMT1
MPQEARTPQQQVAADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 48 - protein, 289 aa, Artificial sequence
  Modified from pMT1
MPQEARTPQQQVGADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 49 - protein, 289 aa, Artificial sequence
  Modified from pMT1
MPQEARTPQQQVTMDEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 50 - protein, 289 aa, Artificial sequence
  Modified from pMT1
MPQEARTPQQQVTRDEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 51 - protein, 289 aa, Artificial sequence
  Modified from pMT1
MPQEARTPQQQVTGDEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 52 - protein, 289 aa, Artificial sequence
  Modified from pMT1
MPQEARTPQQQVTPDEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 53 - protein, 289 aa, Artificial sequence
  Modified from pMT1
MPQEARTPQQQVTLDEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 54 - protein, 289 aa, Artificial sequence
  Modified from pMT1
MPQEARTPQQQVTTDEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 55 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALRAARQRGIAVTGVAVSKEQIAAANRLAAGHGLTERLTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 56 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALVAARQRGIAVTGVAVSKEQIAAANRLAAGHGLTERLTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 57 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKIARQRGIAVTGVAVSKEQIAAANRLAAGHGLTERLTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 58 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKVARQRGIAVTGVAVSKEQIAAANRLAAGHGLTERLTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 59 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKLARQRGIAVTGVAVSKEQIAAANRLAAGHGLTERLTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 60 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVSVSKEQIAAANRLAAGHGLTERLTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 61 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVGVSKEQIAAANRLAAGHGLTERLTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 62 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGNGLTERLTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 63 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGSGLTERLTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 64 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLSERLTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 65 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLEERLTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 66 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTGRLTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 67 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERCTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 68 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERKTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 69 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERVTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 70 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAGRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 71 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAARLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 72 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADADRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 73 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAHRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 74 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADANRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 75 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADASRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 76 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMLRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 77 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGLLLVAEEVVTEELTEPETALFETLFALNVPPRLGEFFDIVSGAGFHTLSLKDLSADLAMTMNVFALGVYSRRVEFTERFGAEFVDGLLAGLGSAQETVIRKTRFFMATLRKPAV

SEQ ID NO: 78 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGLLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 79 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGKLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 80 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGRLLVAEEVVTEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 81 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGDLLVAEEVVVEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 82 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGDLLVAEEVVLEELTEPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 83 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGDLLVAEEVVTEELTQPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 84 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGDLLVAEEVVTEELTAPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 85 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAASHGLTERLTFEVADAMRLPYEDESFDCAWAIESLDHMDRAKALGEAWRVLKPGGDLLVAEEVVTEELTPPETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 86 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGDLLVAEEVVTEELTELETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 87 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGDLLVAEEVVTEELTEIETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 88 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGDLLVAEEVVTEELTEYETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 89 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGDLLVAEEVVTEELTEKETALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 90 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGDLLVAEEVVTEELTEPSTALFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 91 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGDLLVAEEVVTEELTEPETAIFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 92 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGDLLVAEEVVTEELTEPETAVFETLFALNVPPRLGEFFDIVSGAGFHTLSLKDLSADLAMTMNVFALGVYSRRAEFTERFGAEFVDGLLAGLGSAQETVIRKTRFFMATLRKPAV

SEQ ID NO: 93 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGKAQETVIRKTRFFMATLRKPAV

SEQ ID NO: 94 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGQAQETVIRKTRFFMATLRKPAV

SEQ ID NO: 95 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGRAQETVIRKTRFFMATLRKPAV

SEQ ID NO: 96 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGLAQETVIRKTRFFMATLRKPAV

SEQ ID NO: 97 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSLQETVIRKTRFFMATLRKPAV

SEQ ID NO: 98 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSGQETVIRKTRFFMATLRKPAV

SEQ ID NO: 99 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSRQETVIRKTRFFMATLRKPAV

SEQ ID NO: 100 - protein, 289 aa, Artificial sequence
  Modified from pMT1
MPQEARTPQQQVTADEVGDWYDKFGEVYHLTLGESVHCGLWFPPDAPVPQDMELVTMSSQAQDRFTDYLIETLDPKAGQHLLDIGCGTGRPALKAARQRGIAVTGVAVSKEQIAAANRLAAGHGLTERLTFEVADAMRLPYEDESFDCAWAIESLDHMDRAKALGEAWRVLKPGGDLLVAEEVVTEELTEPETALFETLFALNVPPRLGEFFDIVSGAGFHTLSLKDLSADLAMTMNVFALGVYSRRAEFTERFGAEFVDGLLAGLGSYQETVIRKTRFFMATLRKPAV

SEQ ID NO: 101 - DNA, 1476 bp, Artificial sequence
  Modified from pMT1
atgaaactctcaactaaactttgttggtgtggtattaaaggaagacttaggccgcaaaagcaacaacaattacacaatacaaacttgcaaatgactgaactaaaaaaacaaaagaccgctgaacaaaaaaccagacctcaaaatgtcggtattaaaggtatccaaatttacatcccaactcaatgtgtcaaccaatctgagctagagaaatttgatggcgtttctcaaggtaaatacacaattggtctgggccaaaccaacatgtcttttgtcaatgacagagaagatatctactcgatgtccctaactgttttgtctaagttgatcaagagttacaacatcgacaccaacaaaattggtagattagaagtcggtactgaaactctgattgacaagtccaagtctgtcaagtctgtcttgatgcaattgtttggtgaaaacactgacgtcgaaggtattgacacgcttaatgcctgttacggtggtaccaacgcgttgttcaactctttgaactggattgaatctaacgcatgggatggtagagacgccattgtagtttgcggtgatattgccatctacgataagggtgccgcaagaccaaccggtggtgccggtactgttgctatgtggatcggtcctgatgctccaattgtatttgactctgtaagagcttcttacatggaacacgcctacgatttttacaagccagatttcaccagcgaatatccttacgtcgatggtcatttttcattaacttgttacgtcaaggctcttgatcaagtttacaagagttattccaagaaggctatttctaaagggttggttagcgatcccgctggttcggatgctttgaacgttttgaaatatttcgactacaacgttttccatgttccaacctgtaaattggtcacaaaatcatacggtagattactatataacgatttcagagccaatcctcaattgttcccagaagttgacgccgaattagctactcgcgattatgacgaatctttaaccgataagaacattgaaaaaacttttgttaatgttgctaagccattccacaaagagagagttgcccaatctttgattgttccaacaaacacaggtaacatgtacaccgcatctgtttatgccgcctttgcatctctattaaactatgttggatctgacgacttacaaggcaagcgtgttggtttattttcttacggttccggtttagctgcatctctatattcttgcaaaattgttggtgacgtccaacatattatcaaggaattagatattactaacaaattagccaagagaatcaccgaaactccaaaggattacgaagctgccatcgaattgagagaaaatgcccatttgaagaagaacttcaaacctcaaggttccattgagcatttgcaaagtggtgtttactacttgaccaacatcgatgacaaatttagaagatcttacgatgttaaaaaataa

SEQ ID NO: 102 - DNA, 1185 bp, Artificial sequence
  Modified from pMT1
atgaaaaattgtgtcatcgtcagtgcggtacgtactgctatcggtagttttaacggttcactcgcttccaccagcgccatcgacctgggggcgacagtaattaaagccgccattgaacgtgcaaaaatcgattcacaacacgttgatgaagtgattatgggtaacgtgttacaagccgggctggggcaaaatccggcgcgtcaggcactgttaaaaagcgggctggcagaaacggtgtgcggattcacggtcaataaagtatgtggttcgggtcttaaaagtgtggcgcttgccgcccaggccattcaggcaggtcaggcgcagagcattgtggcggggggtatggaaaatatgagtttagccccctacttactcgatgcaaaagcacgctctggttatcgtcttggagacggacaggtttatgacgtaatcctgcgcgatggcctgatgtgcgccacccatggttatcatatggggattaccgccgaaaacgtggctaaagagtacggaattacccgtgaaatgcaggatgaactggcgctacattcacagcgtaaagcggcagccgcaattgagtccggtgcttttacagccgaaatcgtcccggtaaatgttgtcactcgaaagaaaaccttcgtcttcagtcaagacgaattcccgaaagcgaattcaacggctgaagcgttaggtgcattgcgcccggccttcgataaagcaggaacagtcaccgctgggaacgcgtctggtattaacgacggtgctgccgctctggtgattatggaagaatctgcggcgctggcagcaggccttacccccctggctcgcattaaaagttatgccagcggtggcgtgccccccgcattgatgggtatggggccagtacctgccacgcaaaaagcgttacaactggcggggctgcaactggcggatattgatctcattgaggctaatgaagcatttgctgcacagttccttgccgttgggaaaaacctgggctttgattctgagaaagtgaatgtcaacggcggggccatcgcgctcgggcatcctatcggtgccagtggtgctcgtattctggtcacactattacatgccatgcaggcacgcgataaaacgctggggctggcaacactgtgcattggcggcggtcagggaattgcgatggtgattgaacggttgaattaa

SEQ ID NO: 103 - DNA, 1509 bp, Artificial sequence
  Modified from pMT1
atggttttaaccaataaaacagtcatttctggatcgaaagtcaaaagtttatcatctgcgcaatcgagctcatcaggaccttcatcatctagtgaggaagatgattcccgcgatattgaaagcttggataagaaaatacgtcctttagaagaattagaagcattattaagtagtggaaatacaaaacaattgaagaacaaagaggtcgctgccttggttattcacggtaagttacctttgtacgctttggagaaaaaattaggtgatactacgagagcggttgcggtacgtaggaaggctctttcaattttggcagaagctcctgtattagcatctgatcgtttaccatataaaaattatgactacgaccgcgtatttggcgcttgttgtgaaaatgttataggttacatgcctttgcccgttggtgttataggccccttggttatcgatggtacatcttatcatataccaatggcaactacagagggttgtttggtagcttctgccatgcgtggctgtaaggcaatcaatgctggcggtggtgcaacaactgttttaactaaggatggtatgacaagaggcccagtagtccgtttcccaactttgaaaagatctggtgcctgtaagatatggttagactcagaagagggacaaaacgcaattaaaaaagcttttaactctacatcaagatttgcacgtctgcaacatattcaaacttgtctagcaggagatttactcttcatgagatttagaacaactactggtgacgcaatgggtatgaatatgatttctaaaggtgtcgaatactcattaaagcaaatggtagaagagtatggctgggaagatatggaggttgtctccgtttctggtaactactgtaccgacaaaaaaccagctgccatcaactggatcgaaggtcgtggtaagagtgtcgtcgcagaagctactattcctggtgatgttgtcagaaaagtgttaaaaagtgatgtttccgcattggttgagttgaacattgctaagaatttggttggatctgcaatggctgggtctgttggtggatttaacgcacatgcagctaatttagtgacagctgttttcttggcattaggacaagatcctgcacaaaatgttgaaagttccaactgtataacattgatgaaagaagtggacggtgatttgagaatttccgtatccatgccatccatcgaagtaggtaccatcggtggtggtactgttctagaaccacaaggtgccatgttggacttattaggtgtaagaggcccgcatgctaccgctcctggtaccaacgcacgtcaattagcaagaatagttgcctgtgccgtcttggcaggtgaattatccttatgtgctgccctagcagccggccatttggttcaaagtcatatgacccacaacaggaaacctgctgaaccaacaaaacctaacaatttggacgccactgatataaatcgtttgaaagatgggtccgtcacctgcattaaatcctaa

SEQ ID NO: 104 - DNA, 1332 bp, Artificial sequence
  Modified from pMT1
atgagcttaccgttcctgacttcggcaccgggcaaagttatcattttcggcgagcactctgctgtttacaacaaaccggcagttgcggcctccgtatctgcactgcgcacttatctgctgatctctgaaagctccgccccggatactattgaactggactttccggacatttcctttaaccacaaatggagcattaacgactttaacgcgatcactgaagatcaggtaaactcccagaaactggcaaaagcacagcaggctaccgatggtctgagccaggaactggtgtccctcctcgatcctttgctggctcaactctcggaatcgttccattaccatgctgctttctgttttctgtatatgtttgtttgcctctgcccgcacgcgaaaaacatcaaattctctctgaaatcgactctgccgattggtgccggcctgggttcgtccgcatctatttccgtttccctggcgctggccatggcctatctgggcggtctgatcggttccaacgacctggaaaaactctcggaaaacgataagcacatcgttaaccagtgggcgttcatcggtgaaaaatgtatccacggtaccccatccggtatcgataatgcggttgctacctacggtaacgcgttactgttcgaaaaagattctcataacggtactatcaacactaacaacttcaaatttttggacgattttccggcgattccgatgatcctgacttacacccgcatcccgcgtagcaccaaggatctggttgcacgcgttcgtgtactcgtgaccgaaaaattcccggaggttatgaaaccgatcctggatgcaatgggtgagtgcgcgctgcagggattagaaatcatgaccaaactgtcgaagtgtaaaggtacggacgacgaagctgttgaaacaaacaacgaactgtatgaacagctgctggaactgatccgtatcaaccacggcctgctggtcagcattggtgtgagccacccgggcctggaactgattaaaaatctttcggatgacctgcgcattggttctaccaagctgactggtgctggcggcggtggctgttctctgaccctcctgcgtcgcgatattacccaggaacaaatcgactcgttcaaaaaaaaactgcaggatgacttttcttatgaaaccttcgaaaccgacctgggcggtaccggctgttgtctcctgtccgccaaaaacttgaacaaagatctgaaaatcaaatctctcgtctttcagctgtttgaaaacaaaactaccaccaaacaacaaattgatgacctgctgctcccgggcaacaccaacttaccgtggacttcctaa

SEQ ID NO: 105 - DNA, 1356 bp, Artificial sequence
  Modified from pMT1
atgtctgagcttcgcgctttctccgctccgggcaaggccctgctggccggaggctatctggtgctggacaccaaatatgaagcgtttgtagttggtctgtctgcccgtatgcatgcggtcgcgcacccgtatgggtcgctgcagggttcagataaattcgaggtgcgagtgaaaagcaaacagtttaaagacggtgagtggctgtatcacattagcccgaaatctggttttatcccggtatccatcggcggttccaaaaacccgttcattgaaaaagttattgccaacgttttctcctattttaaacctaacatggatgactactgtaaccgtaacctgttcgtgattgatattttctctgacgatgcttaccattcccaggaagacagcgttacggaacaccgtggcaaccgtcgtctgtcgtttcattcccaccgtatcgaagaagttccgaagactggcctgggtagctctgcaggcctggttaccgtcctgactactgctctggcctctttttttgtgtcggatctggaaaacaacgttgacaaatatcgtgaggtaattcataacctggctcaggtcgcacactgccaggcgcagggcaaaatcggctccggtttcgatgttgctgcggcagcttatggctccattcgttaccgtcgcttcccgcctgctctgatctcaaacctgccggatattggtagcgcaacctacggatcgaagctggctcacctggtggatgaggaagattggaatatcaccattaaatctaaccacctgccgtctggcctgaccctgtggatgggtgatatcaaaaacggctctgaaaccgtcaaactggtacagaaagttaagaattggtatgattctcacatgccggaatccctgaaaatctacaccgagctggatcacgcgaactcacgtttcatggacggtctgtccaaactggaccgtctgcacgaaacccacgatgattacagcgaccagatcttcgaatctctggaacgtaacgactgcacctgtcaaaaatacccggaaatcaccgaagttcgtgacgcggtagcgaccatccgccgctctttccgtaaaatcactaaggaaagcggcgctgacatcgaaccgccggttcagacctccctgctggacgattgccagactctgaaaggggtgctgacctgtctgattccgggtgcgggtggttatgacgctatcgcagtgatcacgaaacaggatgtagacctgcgtgcgcagactgcaaacgacaaacgttttagcaaagtacaatggctggatgttacccaggcggattggggtgttcgtaaagaaaaggaccctgaaacctacctggataaataa

SEQ ID NO: 106 - DNA, 1191 bp, Artificial sequence
  Modified from pMT1
atgaccgtttacacagcatccgttaccgcacccgtcaacatcgcaacccttaagtattgggggaaaagggacacgaagttgaatctgcccaccaattcgtccatatcagtgactttatcgcaagatgacctcagaacgttgacctctgcggctactgcacctgagtttgaacgcgacactttgtggttaaatggagaaccacacagcatcgacaatgaaagaactcaaaattgtctgcgcgacctacgccaattaagaaaggaaatggaatcgaaggacgcctcattgcccacattatctcaatggaaactccacattgtctccgaaaataactttcctacagcagctggtttagcttcctccgctgctggctttgctgcattggtctctgcaattgctaagttataccaattaccacagtcaacttcagaaatatcccgtatagcaagaaaggggtctggttcagcttgtagatcgttgtttggcggatacgtggcctgggaaatgggaaaagctgaagatggtcatgattccatggcagtacaaatcgcagacagctctgactggcctcagatgaaagcttgtgtcttagtcgtcagcgatattaaaaaggatgtgagttccactcagggtatgcaattgaccgtggcaacctccgaactatttaaagaaagaattgaacatgtcgtaccaaagagatttgaagtcatgcgtaaagccattgttgaaaaagatttcgccacctttgcaaaggaaacaatgatggattccaactctttccatgccacatgtttggactctttccctccaatattctacatgaatgacacttccaagcgtatcatcagttggtgccacaccattaatcagttttacggagaaacaatcgttgcatacacgtttgatgcaggtccaaatgctgtgttgtactacttagctgaaaatgagtcgaaactctttgcatttatctataaattgtttggctctgttcctggatgggacaagaaatttactactgagcagcttgaggctttcaaccatcaatttgaatcatctaactttactgcacgtgaattggatcttgagttgcaaaaggatgttgccagagtgattttaactcaagtcggttcaggcccacaagaaacaaacgaatctttgattgacgcaaagactggtctaccaaaggaataa

SEQ ID NO: 107 - DNA, 555 bp, Artificial sequence
  Modified from pMT1
atggagctccaaacggaacacgtcattttattgaatgcacagggagttcccacgggtacgctggaaaagtatgccgcacacacggcagacacccgcttacatctcgcgttctccagttggctgtttaatgccaaaggacaattattagttacccgccgcgcactgagcaaaaaagcatggcctggcgtgtggactaactcggtttgtgggcacccacaactgggagaaagcaacgaagacgcagtgatccgccgttgccgttatgagcttggcgtggaaattacgcctcctgaatctatctatcctgactttcgctaccgcgccaccgatccgagtggcattgtggaaaatgaagtgtgtccggtatttgccgcacgcaccactagtgcgttacagatcaatgatgatgaagtgatggattatcaatggtgtgatttagcagatgtattacacggtattgatgccacgccgtgggcgttcagtccgtggatggtgatgcaggcgacaaatcgcgaagccagaaaacgattatctgcatttacccagcttaaataa

SEQ ID NO: 108 - DNA, 924 bp, Artificial sequence
  Modified from pMT1
atggtgagtggcagtaaagcgggcgtttcgcctcatcgcgaaatagaagtaatgagacaatccattgacgatcacctggctggcctgttacctgaaaccgacagccaggatatcgtcagccttgcgatgcgtgaaggcgtcatggcacccggtaaacggatccgtccgctgctgatgctgctggccgcccgcgacctccgctaccagggcagtatgcctacgctgctcgatctcgcctgcgccgttgaactgacccataccgcgtcgctgatgctcgacgacatgccctgcatggacaacgccgagctgcgccgcggtcagcccactacccacaaaaaatttggtgagagcgtggcgatccttgcctccgttgggctgctctctaaagcctttggtctgatcgccgccaccggcgatctgccgggggagaggcgtgcccaggcggtcaacgagctctctaccgccgtgggcgtgcagggcctggtactggggcagtttcgcgatcttaacgatgccgccctcgaccgtacccctgacgctatcctcagcaccaaccacctcaagaccggcattctgttcagcgcgatgctgcagatcgtcgccattgcttccgcctcgtcgccgagcacgcgagagacgctgcacgccttcgccctcgacttcggccaggcgtttcaactgctggacgatctgcgtgacgatcacccggaaaccggtaaagatcgcaataaggacgcgggaaaatcgacgctggtcaaccggctgggcgcagacgcggcccggcaaaagctgcgcgagcatattgattccgccgacaaacacctcacttttgcctgtccgcagggcggcgccatccgacagtttatgcatctgtggtttggccatcaccttgccgactggtcaccggtcatgaaaatcgcctga

SEQ ID NO: 109 - DNA, 930 bp, Artificial sequence
  Modified from pMT1
atgagccaaccgccgctgcttgaccacgccacgcagaccatggccaacggctcgaaaagttttgccaccgctgcgaagctgttcgacccggccacccgccgtagcgtgctgatgctctacacctggtgccgccactgcgatgacgtcattgacgaccagacccacggcttcgccagcgaggccgcggcggaggaggaggccacccagcgcctggcccggctgcgcacgctgaccctggcggcgtttgaaggggccgagatgcaggatccggccttcgctgcctttcaggaggtggcgctgacccacggtattacgccccgcatggcgctcgatcacctcgacggctttgcgatggacgtggctcagacccgctatgtcacctttgaggatacgctgcgctactgctatcacgtggcgggcgtggtgggtctgatgatggccagggtgatgggcgtgcgggatgagcgggtgctggatcgcgcctgcgatctggggctggccttccagctgacgaatatcgcccgggatattattgacgatgcggctattgaccgctgctatctgcccgccgagtggctgcaggatgccgggctgaccccggagaactatgccgcgcgggagaatcgggccgcgctggcgcgggtggcggagcggcttattgatgccgcagagccgtactacatctcctcccaggccgggctacacgatctgccgccgcgctgcgcctgggcgatcgccaccgcccgcagcgtctaccgggagatcggtattaaggtaaaagcggcgggaggcagcgcctgggatcgccgccagcacaccagcaaaggtgaaaaaattgccatgctgatggcggcaccggggcaggttattcgggcgaagacgacgagggtgacgccgcgtccggccggtctttggcagcgtcccgtttag

SEQ ID NO: 110 - DNA, 1461 bp, Artificial sequence
  Modified from pMT1
atgaaaaaaaccgttgtgattggcgcaggctttggtggcctggcgctggcgattcgcctgcaggcggcagggatcccaaccgtactgctggagcagcgggacaagcccggcggtcgggcctacgtctggcatgaccagggctttacctttgacgccgggccgacggtgatcaccgatcctaccgcgcttgaggcgctgttcaccctggccggcaggcgcatggaggattacgtcaggctgctgccggtaaaacccttctaccgactctgctgggagtccgggaagaccctcgactatgctaacgacagcgccgagcttgaggcgcagattacccagttcaacccccgcgacgtcgagggctaccggcgctttctggcttactcccaggcggtattccaggagggatatttgcgcctcggcagcgtgccgttcctctcttttcgcgacatgctgcgcgccgggccgcagctgcttaagctccaggcgtggcagagcgtctaccagtcggtttcgcgctttattgaggatgagcatctgcggcaggccttctcgttccactccctgctggtaggcggcaaccccttcaccacctcgtccatctacaccctgatccacgcccttgagcgggagtggggggtctggttccctgagggcggcaccggggcgctggtgaacggcatggtgaagctgtttaccgatctgggcggggagatcgaactcaacgcccgggtcgaagagctggtggtggccgataaccgcgtaagccaggtccggctggcggatggtcggatctttgacaccgacgccgtagcctcgaacgctgacgtggtgaacacctataaaaagctgctcggccaccatccggtggggcagaagcgggcggcagcgctggagcgcaagagcatgagcaactcgctgtttgtgctctacttcggcctgaaccagcctcattcccagctggcgcaccataccatctgttttggtccccgctaccgggagctgatcgacgagatctttaccggcagcgcgctggcggatgacttctcgctctacctgcactcgccctgcgtgaccgatccctcgctcgcgcctcccggctgcgccagcttctacgtgctggccccggtgccgcatcttggcaacgcgccgctggactgggcgcaggaggggccgaagctgcgcgaccgcatctttgactaccttgaagagcgctatatgcccggcctgcgtagccagctggtgacccagcggatctttaccccggcagacttccacgacacgctggatgcgcatctgggatcggccttctccatcgagccgctgctgacccaaagcgcctggttccgcccgcacaaccgcgacagcgacattgccaacctctacctggtgggcgcaggtactcaccctggggcgggcattcctggcgtagtggcctcggcgaaagccaccgccagcctgtga

SEQ ID NO: 111 - DNA, 906 bp, Artificial sequence
  Modified from pMT1
atggagctcgactttccgcagcaactcgaagcctgcgttaagcaggccaaccaggcgctgagccgttttatcgccccactgccctttcagaacactcccgtggtcgaaaccatgcagtatggcgcattattaggtggtaagcgcctgcgacctttcctggtttatgccaccggtcatatgttcggcgttagcacaaacacgctggacgcacccgctgccgccgttgagtgtatccacgcttactcattaattcatgatgatttaccggcaatggatgatgacgatctgcgtcgcggtttgccaacctgccatgtgaagtttggcgaagcaaacgcgattctcgctggcgacgctttacaaacgctggcgttctcgattttaagcgatgccgatatgccggaagtgtcggaccgcgacagaatttcgatgatttctgaactggcgagcgccagtggtattgccggaatgtgcggtggtcaggcattagatttagacgcggaaggcaaacacgtacctctggacgcgcttgagcgtattcatcgtcataaaaccggcgcattgattcgcgccgccgttcgccttggtgcattaagcgccggagataaaggacgtcgtgctctgccggtactcgacaagtatgcagagagcatcggccttgccttccaggttcaggatgacatcctggatgtggtgggagatactgcaacgttgggaaaacgccagggtgccgaccagcaacttggtaaaagtacctaccctgcacttctgggtcttgagcaagcccggaagaaagcccgggatctgatcgacgatgcccgtcagtcgctgaaacaactggctgaacagtcactcgatacctcggcactggaagcgctagcggactacatcatccagcgtaataaataa

SEQ ID NO: 112 - DNA, 1968 bp, Artificial sequence
  Modified from pMT1
atgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggcgggtcgtaaagaaagcgacgacggcgttgaacgcatcgaaggtggcgtggttgttgtaaacccaaaaccaaagaaaggcatcactgcatcgaaagcaattgactggctggagtggctgtttgttaaaatgatgcatgacagcaaacagccgctgcactacttgtccggtaacttcgcaccggttgacgaaaccccgccgctgaaagacctgccggtgaccggccatctgccggaatgcctgaacggcgagtttgtgcgtgtcggtccgaacccgaaattcgcttccatcgccggttaccactggtttgacggtgatggtatgatccatggtatgcgcattaaggacggtaaagccacctatgtttctcgttatgttcagacctcccgcctcaaacaggaagaattcttcggtcgtgctatgtttatgaaaattggagatctgaaaggtatgttcggcctgttcaccgtttacatgcagatgctgcgtgcgaaactgaaagtcctggacgcatcttacggtattggtaccgccaacaccgcgctggtttaccaccatggcaaactgctcgctctgagcgaagctgacaaaccgtatgcgatcaaagttctggaagatggcgacctgcagaccatcggcctgttagattacgacaaacgcctggcacatagtttcaccgcacatcccaaggttgacccgtttacaggcgaaatgttcacgttcggttattcccacaccccgccgtatgtgacctaccgtgttatctctaaagatggtgcaatgaacgatccggtacctatcacggtctccggcccaatcatgatgcacgacttcgccatcacggaaaactacgctattttcatggatttgccgctgtacttcaaaccaaaagagatggtgaaagataaaaagttcatcttcagcttcgacgcaacccagaaggctcgctttggcatcctgccgcgctacgccaaaaacgaatctctgatcaaatggttcgagctcccgaactgcttcatctttcacaatgcgaacgcttgggaagagggtgacgaagttgtccttattacctgccgtctggaaaacccggacctggacatggttaacagcaccgttaaagaacgtctggacaacttcaaaaacgaactgtacgaaatgcgcttcaacctgcagaacggtctggctagccagaaaaaactgagcgtaagcgcggttgattttccgcgtgttaacgaatcgtacaccacccgtaaacagcgttatgtttatggtacgaccctggataacatcgcgaaagttaccggtatcatcaaattcgacctgcacgccgaaccggaaacgggcaaagaaaagctggaactgggtggcaacgtaaaaggtatttttgatctgggcccgggtcgcttcggtagtgaagccgttttcgttccacgtcacccgggcatcacctccgaagaagatgacggttatctcatttttttcgtgcatgatgagaacacaggtaaatctgcagtaaacgtgatcgacgcaaaaaccatgtctcctgacccggttgcagtagtcgaactgccgaaacgcgtgccatacggctttcacgcttttttcgtaaccgaagaccagctgcaggaacaggccaaagtgtaa

SEQ ID NO: 113 - DNA, 1155 bp, Artificial sequence
  Modified from pMT1
atggcaaaacacctttttacgtccgagtccgtctctgaagggcatcctgacaaaattgctgaccaaatttctgatgccgttttagacgcgatcctcgaacaggatccgaaagcacgcgttgcttgcgaaacctacgtaaaaaccggcatggttttagttggcggcgaaatcaccaccagcgcctgggtagacatcgaagagatcacccgtaacaccgttcgcgaaattggctatgtgcattccgacatgggctttgacgctaactcctgtgcggttctgagcgctatcggcaaacagtctcctgacatcaaccagggcgttgaccgtgccgatccgctggaacagggcgcgggtgaccagggtctgatgtttggctacgcaactaatgaaaccgacgtgctgatgccagcacctatcacctatgcacaccgtctggtacagcgtcaggctgaagtgcgtaaaaacggcactctgccgtggctgcgcccggacgcgaaaagccaggtgacttttcagtatgacgacggcaaaatcgttggtatcgatgctgtcgtgctttccactcagcactctgaagagatcgaccagaaatcgctgcaagaagcggtaatggaagagatcatcaagccaattctgcccgctgaatggctgacttctgccaccaaattcttcatcaacccgaccggtcgtttcgttatcggtggcccaatgggtgactgcggtctgactggtcgtaaaattatcgttgatacctacggcggcatggcgcgtcacggtggcggtgcattctctggtaaagatccatcaaaagtggaccgttccgcagcctacgcagcacgttatgtcgcgaaaaacatcgttgctgctggcctggccgatcgttgtgaaattcaggtttcctacgcaatcggcgtggctgaaccgacctccatcatggtagaaactttcggtactgagaaagtgccttctgaacaactgaccctgctggtacgtgagttcttcgacctgcgcccatacggtctgattcagatgctggatctgctgcacccgatctacaaagaaaccgcagcatacggtcactttggtcgtgaacatttcccgtgggaaaaaaccgacaaagcgcagctgctgcgcgatgctgccggtctgaagtaa

SEQ ID NO: 114 - DNA, 699 bp, Artificial sequence
  Modified from pMT1
atgaaaatcggcatcattggtgcaatggaagaagaagttacgctgctgcgtgacaaaatcgaaaaccgtcaaactatcagtctcggcggttgcgaaatctataccggccaactgaatggaaccgaggttgcgcttctgaaatcgggcatcggtaaagtcgctgcggcgctgggtgccactctgctgttggaacactgcaagccagatgtgattattaacaccggttctgccggtggcctggcaccaacgttgaaagtgggcgatatcgttgtctcggacgaagcacgttatcacgacgcggatgtcacggcatttggttatgaatacggtcagttaccaggctgtccggcaggctttaaagctgacgataaactgatcgctgccgctgaggcctgcattgccgaactgaatcttaacgctgtacgtggcctgattgttagcggcgacgctttcatcaacggttctgttggtctggcgaaaatccgccacaacttcccacaggccattgctgtagagatggaagcgacggcaatcgcccatgtctgccacaatttcaacgtcccgtttgtcgtagtacgcgccatctccgacgtggccgatcaacagtctcatcttagcttcgatgagttcctggctgttgccgctaaacagtccagcctgatggttgagtcactggtgcagaaactggcacatggctaa

(19) United States
(12) Patent Application Publication
(10) Pub. No.: US 2024/0240209 A1
(43) Pub. Date: Jul. 18, 2024

(54) SYNTHESIS OF ENANTIOPURE CIS-A-IRONE FROM A RENEWABLE CARBON SOURCE

(71) Applicants: Agency for Science, Technology and Research, Singapore (SG); Institut National De Recherche Pour L' Agriculture, L'Alimentation Et L'Environment, Paris (FR); Centre National De La Recherche Scientifique, Paris (FR); Institut National Des Sciences Appliquées De Toulouse, Toulouse (FR)

(72) Inventors: Xixian CHEN, Singapore (SG); Congqiang ZHANG, Singapore (SG); Rehka T, Singapore (SG); Sudha SHUKAL, Singapore (SG); Derek SMITH, Singapore (SG); Isabelle ANDRÉ, Toulouse (FR); Esque JEREMY, Toulouse (FR)

(21) Appl. No.: 18/556,152

(22) PCT Filed: Apr. 20, 2022
(86) PCT No.: PCT/SG2022/050235

§ 371 Date: Oct. 19, 2023

(30) Foreign Application Priority Data
Apr. 20, 2021 (SG) 10202104047X

(51) Int. Cl. C12P 7/26; C12N 9/10; C12N 9/78; C12N 15/52; C12N 15/70; C12P 41/00

(52) U.S. Cl. CPC C12P 7/26; C12N 9/1007; C12N 9/78; C12N 15/52; C12N 15/70; C12P 41/00; C12Y 201/01; C12Y 201/01067; C12Y 305/04028; C12N 2800/101

(57) ABSTRACT

Synthesis of enantiopure cis-α-irone from a renewable carbon source Disclosed herein are natural and synthetic enzymes capable of performing a method of producing cis-α-irone. The method comprises providing an enzyme capable of converting psi-ionone to cis-α-irone; and contacting the enzyme and psi-ionone under suitable conditions to produce cis-α-irone. The enzyme may be a methyltransferase from Streptomyces albireticuli (SaMT), a promiscuous bifunctional methyltransferase/cyclase (pMT1) enzyme from Streptomyces, or a modified pMT1 enzyme with at least one substitution. The enzymes allow the in-vivo and in-vitro production of cis-α-irone including the use of glucose as feedstock for the biotransformation into cis-α-irone.

The present application claims priority to Singapore patent application number 10202104047X filed on 20 Apr. 2021 which is incorporated by reference herein in its entirety.

### BACKGROUND

Genetically engineered microorganisms have been rapidly developed as the workhorse to produce essential molecules at unprecedented yield¹. Technological advancement has enabled simultaneous optimization of multiple genes in a single microbial strain. To date, multiple genes can be rapidly assembled and transformed into microbial strains to probe the collective functions of the set of genes. In this manner, many complex and valuable natural products can be produced. However, one important prerequisite is the availability of prior knowledge about the product's biosynthetic pathway. Unfortunately for many industrially important molecules, their biosynthetic pathways are often incomplete with many missing enzymatic reactions.

Cis-α-irone, the principal component of orris root oil, is a good example. Traditionally used in “Queen's water”, orris oil commands a hefty price in the fragrance industry due to its lengthy and inefficient manufacturing process. With a production period spanning over three years, approximately 30-70 mg of natural irone can be produced from 1 kg of fresh iris rhizome. In the 1990s, Gil and co-workers patented an enzymatic process to degrade irone precursors from orris roots with lipoxidase or peroxidase². The method improved the production rate but was still reliant on plant materials. Furthermore, different species of iris give rise to divergent odor perceptions, because the composition of irone isomers are non-identical^{3,4} as there are 10 different regio- and stereoisomers of irone⁵. GC-Olfactory analysis of α-irone mixtures had been performed (FIG. 6), and the results agree with previous reports indicating that cis-α-irones are olfactive but not trans-α-irones^{3,5}. A semi-synthetic method exists to produce irones (Irone Alpha® by Givaudan) from methyl psi-ionone (FIG. 1), a non-natural substrate. Irone Alpha is a racemic mixture containing 42% cis-α-irones ((1S,5R)-cis-α-irone (1S5R) and (1R,5S)-cis-α-irone (1R5S)), 53% trans-α-irones ((1R,5R)-trans-α-irone (1R5R) and (1S,5S)-trans-α-irone (1S5S)) and 5% β-irones⁵. Similarly, GC analysis showed synthetic irones differ significantly from natural irones, in which the cis-isomers are the predominant compounds (FIG. 6). Lipase-mediated synthesis can resolve the racemic mixture of Irone Alpha³. However, the process involves more than 5 steps of oxidation and reduction, exacerbating the purity of the final product. Total enzymatic synthesis of cis-α-irone through microbial fermentation could potentially eliminate the reliance on orris rhizomes and shorten the production process, but it is not yet feasible because multiple unknown enzymes along the irone biosynthetic pathway hinder the engineering effort (FIG. 1). A radio-labelling study in the 1980s demonstrated that irones are oxidative degradation products of a triterpenoid, iridal (FIG. 1)⁶. According to the study, it was proposed that the irone moiety is formed by a bifunctional methyltransferase/cyclase (bMTC). However, till now, this plant enzyme has not been identified.

### SUMMARY

Artificial biosynthetic routes could be designed by leveraging on promiscuous enzymes to overcome the problem of identifying the specific enzyme/s in the natural biosynthetic pathway

In a first aspect of the invention, there is provided a modified enzyme comprising a first substitution of base SEQ ID No. 3 at a position selected from the group consisting of position 200, position 180, position 160, and position 236, wherein if the first substitution is at position 200, the first substitution is selected from the group consisting of phenylalanine, isoleucine, leucine, valine, and tryptophan, wherein if the first substitution is at position 180, the first substitution is selected from the group consisting of alanine, cysteine, glutamic acid, isoleucine, methionine, and valine, wherein if the first substitution is at position 160, the first substitution is selected from the group consisting of alanine, cysteine, histidine, isoleucine, leucine, methionine, asparagine, glutamine, threonine, valine, and tyrosine, wherein if the first substitution at position 236 is selected, the first substitution is selected from the group consisting of cysteine, glutamic acid, histidine, isoleucine, leucine, asparagine, serine, threonine, and valine. More preferably, the first substitution is at position 200, position 180, or position 160 as described above.

Preferably, the modified enzyme comprises a second substitution selected from the group consisting of position 182 and position 180, wherein if the second substitution is at position 182, the second substitution is selected from the group consisting of glutamic acid, threonine, asparagine and glutamine, wherein the second substitution may be selected at position 180 only if the first substitution is not at position 180, and if the second substitution is at position 180, the second substitution is selected from the group consisting of alanine, cysteine. In an embodiment, the first substitution is at position 200 and the second substitution is at position 182.

Preferably, the modified enzyme comprises a third substitution at position 273, wherein the third substitution is selected from the group consisting of valine, isoleucine, and lysine. In an embodiment, the first, second, and third substitutions are at positions 200, 182, and 273 respectively.

Preferably, the modified enzyme comprises a fourth substitution at position 180, wherein the first substitution is not at position 180 and the second substitution if present is not at position 180, and the fourth substitution is alanine or cysteine. In an embodiment, the first, second, third, and fourth substitutions are at positions 200, 182, 273, and 180 respectively.

Preferably, the modified enzyme comprises a fifth substitution at position 202 to a bulkier amino acid. For example, the bulkier amino acid may be selected from the group consisting of leucine, valine, and phenylalanine. In an embodiment, the first, second, third, fourth, and fifth substitutions are at positions 200, 182, 273, 180, and 202 respectively.

Preferably, the modified enzyme comprises a sixth substitution at position 65, wherein the sixth substitution is selected from the group consisting of phenylalanine, leucine, and methionine. In an embodiment, the first, second, third, fourth, fifth and sixth substitutions are at positions 200, 182, 273, 180, 202 and 65 respectively.

Preferably, the modified enzyme comprises a seventh substitution at position 156, wherein the seventh substitution is selected from the group consisting of aspartic acid, alanine, proline, glycine, and serine. In an embodiment, the first, second, third, fourth, fifth and seventh substitutions are at positions 200, 182, 273, 180, 202 and 156 respectively. In an embodiment, the first, second, third, fourth, fifth, sixth, and seventh substitutions are at positions 200, 182, 273, 180, 202, 65 and 156 respectively.

Preferably, the modified enzyme comprises an eighth substitution at position 91, wherein the eighth substitution is proline. In an embodiment, the first, second, third, fourth, fifth, sixth, seventh, and eighth substitutions are at positions 200, 182, 273, 180, 202, 65, 156, and 91 respectively.

Preferably, the modified enzyme comprises a ninth substitution at position 231, wherein the ninth substitution is aspartic acid. In an embodiment, the first, second, third, fourth, fifth, sixth, seventh, eighth, and ninth substitutions are at positions 200, 182, 273, 180, 202, 65, 156, 91, and 231 respectively.

Preferably, the modified enzyme comprises a tenth substitution selected from the group consisting of alanine at position 244, alanine at position 245, and proline at position 267. More preferably, the modified enzyme comprises at least two of the three, even more preferably all three substitutions.

Preferably, the modified enzyme comprises an eleventh substitution at position 197, wherein the eleventh substitution is arginine.

Preferably, the modified enzyme comprises a twelfth substitution at position 60, wherein the twelfth substitution is selected from the group consisting of valine, lysine, and arginine. The twelfth substitution may be considered as a seventh substitution described above. In an embodiment, the first, second, third, fourth, fifth, sixth and twelfth substitutions are at positions 200, 182, 273, 180, 202, 65, and 60 respectively.

Preferably, the modified enzyme comprises a thirteenth substitution selected from the group consisting of: (i) the thirteenth substitution is at position 11 and selected from histidine, leucine, proline, methionine, valine, and tryptophan; (ii) the thirteenth substitution is at position 12 and selected from lysine, alanine, glycine, and arginine; (iii) the thirteenth substitution is at position 13 and selected from leucine, methionine, glutamine, alanine, and glycine; (iv) the thirteenth substitution is at position 14 and selected from methionine, arginine, glycine, proline, leucine, and threonine; (v) the thirteenth substitution is at position 94 and selected from arginine, and valine; (vi) the thirteenth substitution is at position 95 and selected from isoleucine, cysteine, valine, and leucine; (vii) the thirteenth substitution is at position 107 and selected from serine and glycine; (viii) the thirteenth substitution is at position 123 and selected from asparagine, glutamine, and serine; (ix) the thirteenth substitution is at position 126 and selected from serine and glutamic acid; (x) the thirteenth substitution is at position 127 and selected from glycine and threonine; (xi) the thirteenth substitution is at position 129 and selected from cysteine, lysine, and valine; (xii) the thirteenth substitution is at position 137 and selected from glycine, alanine, aspartic acid, histidine, asparagine, serine, and threonine; (xiii) the thirteenth substitution is at position 159 and is leucine; (xiv) the thirteenth substitution is at position 176 and selected from leucine, valine, lysine, arginine, and tyrosine, and optionally a fourteenth substitution of valine at position 248, with preferably the thirteenth substitution being leucine; (xv) the thirteenth substitution is at position 185 and selected from valine and leucine; (xvi) the thirteenth substitution is at position 190 and selected from glutamine, alanine and proline, and optionally a fifteenth substitution of serine at position 122; (xvii) the thirteenth substitution is at position 191 and selected from serine, valine, leucine, isoleucine, tyrosine, and lysine; (xviii) the thirteenth substitution is at position 192 and selected from serine; (xix) the thirteenth substitution is at position 195 and selected from isoleucine, and valine; (xx) the thirteenth substitution is at position 212 and is leucine; (xxi) the thirteenth substitution is at position 268 and selected from lysine, glutamine, histidine, arginine, and leucine; (xxii) the thirteenth substitution is at position 269 and selected from leucine, glycine, arginine, and tryptophan; and (xxiii) the thirteenth substitution is at position 272 and is alanine. The thirteenth substitution may be considered as a tenth substitution described above. In an embodiment, the first, second, third, fourth, fifth, sixth, seventh, eighth, ninth and thirteenth substitutions are at positions 200, 182, 273, 180, 202, 65, 156, 91, 231 and as described above for the thirteenth substitution respectively.

Preferably, the modified enzyme comprises a sequence selected from the group consisting of SEQ ID No. 15, SEQ ID No. 14, SEQ ID No. 13, SEQ ID No. 16, SEQ ID No. 12, SEQ ID No. 11, SEQ ID No. 10, SEQ ID No. 9, SEQ ID No. 8, SEQ ID No. 7, SEQ ID No. 6, SEQ ID No. 5, SEQ ID No. 4, and SEQ ID No. 20 to 100.

Preferably, the modified enzyme comprises a polyhistidine-tag.

In a second aspect of the invention, there is provided a method of producing cis-α-irone, the method comprises providing an enzyme capable of converting psi-ionone to cis-α-irone; and contacting the enzyme and psi-ionone under suitable conditions to produce cis-α-irone.

Preferably, the enzyme is selected from the group consisting of a modified enzyme, SEQ ID No. 1, and SEQ ID No. 3, the modified enzyme being as described above for the first aspect of the invention.

Preferably, contacting the modified enzyme and psi-ionone is done in the presence of an auxiliary enzyme that removes or recycles SAH. More preferably, the auxiliary enzyme is selected from the group consisting of S-adenosylmethionine synthase (MetK), adenosylhomocysteinase (SAH1), 5′-methylthioadenosine/S-adenosylhomocysteine nucleosidase (mtn), 5-methylthioadenosine/S-adenosylhomocysteine deaminase (mtaD), and halide methyl transferase (HMT).

Preferably, psi-ionone has a maximum concentration of 5 mM.

Preferably, the modified enzyme is provided as a lysate of a cell expressing the modified enzyme or a host cell comprising a plurality of nucleic acid sequences to encode at least one host cell enzyme and a first nucleic acid sequence encoding the modified enzyme, wherein the plurality of enzymes are produced by the host cell to assist in converting glucose or glycerol to psi-ionone.

Preferably, the host cell enzyme includes at least one of the following: HMG-COA synthase (HmgS), Acetoacetyl-CoA thiolase (AtoB), truncated HMG-COA reductase (tHmgR) or HMG-CoA reductase, mevalonate kinase (MevK), phosphomevalonate kinase (PMK), mevalonate pyrophosphate decarboxylase (PMD), IPP isomerase (Idi), GGPP synthase (CrtE), phytoene synthase (CrtB), phytoene desaturase (Crtl), FPP synthase (IspA), and modified OfCCD1 enzyme or OfCCD1 enzyme.

Preferably, at least one of the following conditions are fulfilled: (i) the host cell further comprises a second nucleic acid sequence to encode SAM cycle enzymes; (ii) a third nucleic acid sequence encoding a transcription regulator metJ is absent in the host cell; (iii)

the host cell further comprises at least one T7 promoter sequence; (iv) the host cell is Escherichia coli.

Preferably, the modified enzyme is provided by the host cell and the suitable conditions include a dissolved oxygen content of 30% or less, preferably 1% to 15%, more preferably 2% to 10%.

Preferably, at least 0.1 μg/L of cis-α-irone is produced or a minimum cis-to-trans α-irone ratio of 2 to 5.

In a third aspect of the invention, there is provided a host cell comprising a plurality of nucleic acid sequences to encode enzymes to allow the host cell to convert glucose or glycerol to psi-ionone and a first nucleic acid sequence encoding the modified enzyme according to the first aspect of the invention. Preferably, the enzymes include at least one of the following: HMG-COA synthase (HmgS), Acetoacetyl-CoA thiolase (AtoB), truncated HMG-COA reductase (tHmgR) or HMG-COA reductase, mevalonate kinase (MevK), phosphomevalonate kinase (PMK), mevalonate pyrophosphate decarboxylase (PMD), IPP isomerase (Idi), GGPP synthase (CrtE), phytoene synthase (CrtB), phytoene desaturase (Crtl), FPP synthase (IspA), and modified OfCCD1 enzyme or OfCCD1 enzyme. More preferably, most of the enzymes are included, even more preferably all the enzymes are included.

Preferably, at least one of the following conditions is fulfilled: (i) the host cell further comprising a second nucleic acid sequence to encode SAM cycle enzymes; (ii) a third nucleic acid sequence encoding a transcription regulator metJ is absent (in the host cell); (iii) the host cell further comprising at least one T7 promoter sequence; (iv) the host cell is Escherichia coli

In a fourth aspect of the invention, there is provided a method of methylating a hydroxyl group, the method comprising providing an enzyme selected from the group consisting of the modified enzyme according to the first aspect of the invention (preferably SEQ ID No. 8), SEQ ID No. 1 and SEQ ID No. 3, or the host cell according to the third aspect of the invention; and contacting the enzyme or the host cell and a hydroxyl group under suitable conditions to methylate the hydroxyl group. Preferably, the hydroxyl group is an allylic hydroxyl group.

In all aspects of the invention, the modified enzyme comprises, or consists essentially of, or consists of, one or more of the substitution/s or SEQ ID No as described herein in the specification. For example, the modified enzyme may comprise, or consists essentially of, or consists of, the first substitution of SEQ ID No. 2, and optionally any additional substitutions as described. For example, the modified enzyme may comprise, or consists essentially of, or consists of, any one of the SEQ ID No. recited above. In the specific examples described herein, whilst substitutions at certain positions of SEQ ID No. 2 is in combination with other substitutions, it is likely that these substitutions alone would have similar effect on improving the selectivity and/or yield of cis-α-irone.

Features that are described in the context of an embodiment may correspondingly be applicable to the same or similar features in the other embodiments. Features that are described in the context of an embodiment may correspondingly be applicable to the other embodiments, even if not explicitly described in these other embodiments. Furthermore, additions and/or combinations and/or alternatives as described for a feature in the context of an embodiment may correspondingly be applicable to the same or similar feature in the other embodiments.

The following detailed description is merely exemplary in nature and is not intended to limit the invention or the application and uses of the invention. Furthermore, there is no intention to be bound by any theory presented in the preceding background of the invention or the following detailed description.

Other technical advantages may become readily apparent to one of ordinary skill in the art after review of the following figures and description.

### DESCRIPTION OF FIGURES

Figure (FIG. 1 shows schematic representations of the different pathways to produce irone.

FIG. 2a shows the results of selected methyltransferases (pMT1, SaMT, and ScMT) tested for their promiscuous activities to convert psi-ionone to irone.

FIG. 2b shows the quantification of cis-α-irone, trans-α-irone and β-irone detected for each pMT mutant by HS-SPME-GCMS against an external standard curve. Bar chart represents the average fold change in cis-α-irone concentration as compared to pMT1 over replicated reactions. The ratio between cis-α-irone to trans-α-irone concentration was calculated. The percentage of cis-α-irone in the irone mixture was also calculated by diving cis-α-irone concentration by the total irone concentration. Data shown is an average of replicate experiments.

FIG. 2c shows the crystallographic structure of hexameric pMT1 in complex with a cofactor S-adenosyl-I-homocysteine (yellow sticks) and the substrate teleocidin A1 (pink sticks) (PDB id: 5GM2). Enlarged view of amino acid residues lining the active site.

FIGS. 2d and 2e show the superposition of 3D models of complexes of pMT1 with d, cis-α-irone (1S5R and 1R5S in magenta color) and e, trans-α-irone (1S5S and 1R5R in green color) respectively. pMT1 is shown as cartoon with one chain in cyan color and the second chain in grey color. The 24 amino acid positions selected for site mutagenesis are displayed as orange sticks. Labels of residues from the second chain are underlined.

FIG. 3 shows the kinetic characterization of pMT7 and pMT10 enzymes. FIG. 3a shows the fold change in pMT7 activity when S-adenosyl-L-homocysteine (SAH) (0-200 μM), homocysteine (0-200 μM), adenine (0-200 μM), or adenosine (0-200 μM) was supplemented into purified pMT7 reaction. It was calculated by dividing the pMT7 activity where no additive was added. FIG. 3b shows the fold change in pMT7 and pMT10 activities when SAH (0-40 μM) was supplemented into the purified enzyme reactions. It was calculated by dividing the pMT7 and pMT10 activity where no additive was added. FIG. 3c shows the effect of introducing auxiliary enzymes, mtn and mtaD, to hydrolyze SAH on cis-α-irone production by purified pMT7 and pMT10 with or without initial 20 UM SAH. pMT10 produced ˜4.5 and ˜6.5-fold higher amount of cis-α-irone without or with mtaD enzyme, respectively as compared to pMT7 reaction alone. All the purified enzyme reactions were done in triplicates. FIG. 3d shows the in vitro biotransformation of psi-ionone to cis-α-irone by using cell lysates containing overexpressed pMT10. The bar chart represents the mean value of replicated reactions.

FIG. 4 shows the optimizing of in vivo production of cis-α-irone from renewable carbon source. FIG. 4a shows a schematic representation of the plasmids used to produce cis-α-irone (Table 5). Four plasmids were used. The first plasmid carried the SAR module, which contained the upper mevalonate enzymes: HMG-COA synthase (HmgS), acetoacetyl-CoA thiolase (AtoB) and truncated HMG-COA reductase (tHmgR), and engineered OfCCD1 fused with thioredoxin (TofCCD1m)⁷. The second plasmid carried the MPPI module, which contained the lower mevalonate enzymes: mevalonate kinase (MevK), phosphomevalonate kinase (PMK), mevalonate pyrophosphate decarboxylase (PMD) and isopentenyl pyrophosphate (IPP) isomerase (Idi). The third plasmid carried the lycopene synthesis EBIA module: geranylgeranyl pyrophosphate (GGPP) synthase (CrtE), phytoene synthase (CrtB), phytoene desaturase (Crtl) and farnesyl pyrophosphate (FPP) synthase (IspA). The fourth plasmid carried the pMT10 enzyme or pMT10 with the SAM cycle enzymes: S-adenosylmethionine synthase (MetK) and S-adenosylhomocysteine nucleosidase (mtn). Tm1, Tm2, Tm3 are the mutated T7 promoters with different strength (Tm1>Tm2>Tm3)¹⁵. FIG. 4b shows a schematic representation of the pathway to produce cis-α-irone from glucose or glycerol. SAM recycle enzymes (MetK and mtn), and simplified regulation by metJ are shown. Dotted line represents the cell membrane. FIGS. 4c, 4d, 4e, and 4f show bar charts respectively representing the average amount of cis-α-irone produced over triplicated reaction, the average amount of psi-ionone remained over triplicated reaction, the average specific titer of cis-α-irone produced over triplicated reaction, and the average specific titer of psi-ionone remained over triplicated reaction. Refer to Table 5 for strain description. Three conditions were tested for in vivo α-irone production—0 mM L-Met: no methionine or dodecane was added; 10 mM L-Met: 10 mM methionine but no dodecane was added; 10 mM L-Met wDC: 10 mM methionine with 20% v/v dodecane were added.

FIG. 5 shows the results of the fed-batch production process of cis-α-irone. FIGS. 5a and 5b show the time-course profiles of OD₆₀₀, cis-α-irone, β-irone and psi-ionone titers when DO was controlled at 2% and 10% respectively. For both bioprocesses, the production was induced when OD₆₀₀ reached 30-40 and glucose feeding was stopped at 113 h.

FIG. 6 shows the headspace solid-phase microextraction coupled to gas chromatography-olfactory (HS-SPME-GCO) analysis to identify the olfactory property of irone isomers. The black trace is the GC peaks and the red line (rectangular shaped) indicates if aroma is detected. Clearly, only cis-α-irone has olfactory properties whereas trans-α-irone does not have any olfactory properties. The structures of the four α-irone isomers are shown.

FIG. 7 shows the headspace solid-phase microextraction coupled to gas chromatography-time-of-flight mass spectrometry (HS-SPME-GCMS) analysis to identify the initial methyltransferase activity. FIG. 7a shows the results of the cell lysates overexpressing IspD (negative control) or pMT1 were used to assay against psi-ionone. The reaction mixtures were incubated at 28° C. for 2 days and subjected to HS-SPME-GCMS analysis. Methylated products that correspond to trans-α-irone and cis-α-irone were detected in the reaction containing pMT1. The retention time and mass spectrum of the irones detected were the same as the synthetic chemical standard (std) from Sigma Aldrich. FIG. 7b shows the GC analysis of the reaction mixtures containing cell lysates overexpressing IspD (control), SaMT, ScMT and pMT1-Y200L. Methylated products that correspond to trans-α-irone and cis-α-irone were detected in the headspace of reaction by SaMT and pMT1 with Y200L mutation. The retention time of the irones are the same as the synthetic chemical standard.

FIG. 8 shows a schematic representation and results of the site-saturation mutagenesis and pooled-colony reactions to identify the desired residues to mutate.

FIG. 9 shows the directed evolution of pMT1 to produce cis-α-irone. FIG. 9a shows the Western blot analysis of the soluble amount of pMT mutant enzymes taken from an equal volume of cell lysate. Lanes 1 and 16 are the molecular marker. Lanes 2 to 7 are the purified known amount of pMT1 (2-fold dilution from 90 ng/μl to 2.8 ng/μl) for quantification purpose. The signal corresponding to pMT enzymes is indicated by the arrowhead. FIG. 9b shows a bar chart representing the mean total turnover number (TTN) of each pMT mutant over replicated reactions. The TTN was obtained by diving the concentration of cis-α-irone produced by the concentration of soluble pMT enzyme in the cell lysate. The fold change in TTN of the mutants was measured against the TTN of pMT1.

FIG. 10 shows the structural visualization of the mutant S182E (pMT3) and the mutation study to explore the effect of S182E mutation. FIG. 10a shows the hydrogen bond (dashed line) was formed between the protonated E182 residue and the ketone moiety of cis-α-irone (both 1R5S and 1S5R shown together). Cis-α-irone was manually docked into the active site of pMT3 with energy minimization and the snapshot corresponds to the beginning of MD simulation. FIG. 10b shows that when F196 was mutated to R196, it competed with irone for hydrogen bonding with E182, thus the activity of enzyme was negatively affected. FIG. 10c shows a bar chart representing the level of trans- and cis-α-irone detected in the headspace of the reaction catalyzed by a negative (−ve) control (ispD), pMT2 (Y200F), pMT2-S182D (Y200F_S182D), and pMT3 (Y200F_S182E). When S182 was mutated to Asp, hardly any cis-α-irone was detected, whereas when S182 was mutated to Glu, significant increase in cis-α-irone was detected. FIG. 10d shows a bar chart representing the level of trans- and cis-α-irone detected in the headspace of the reaction catalyzed by a negative control (ispD), pMT3 (Y200F_S182E) and pMT3-F196R (Y200F_S182D_F196R). Cis-α-irone level was significantly decreased when F196 was mutated to Arg on top of pMT3.

FIG. 11 shows the structural visualization of active residue E153 based on pMT3. FIG. 11a shows a pMT3 model as a cartoon representation. The residues E153, Y65, irone isomers and SAH cofactors are displayed as sticks. The interaction network for the reactivity is highlighted by dashed lines, where the distances are on average 4-5 Å between the C4 carbon and the Sulphur of SAH or the oxygen of E153. The hydrogen bond between Y65 and E153 is around 2.0 Å. The snapshot was taken from the beginning of the MD simulation. FIG. 11b shows the chiral GC analysis of the α-irone isomers from a synthetic (chemical) standard, orris root oil, reactions by pMT3 and ispD (negative control). The peaks corresponding to the four α-irone isomers were labelled. Clearly, all four isomers are present in the chemical standard. Three isomers were detected in orris root oil with 1S5R and 1R5S being the major irone constituents, and 1S5S as the minor irone constituent. Only two irone isomers (1R5S and 1S5S) were detected in the reaction by pMT3, with 1R5S (the finest iris-like note) being the predominant product.

FIG. 12 shows the structural visualization of the interaction between L273 and irone and mutagenesis to identify the best mutant at L273. FIG. 12a shows the interactions between the four isomers of irone and L273. The cis isomers (1R5S and 1S5R) are colored as magenta gradient whereas the trans isomers (1R5R and 1S5S) are in green gradient. E182, F200 and L273 form a structural environment to interact with irone, mostly the trans isomers. FIG. 12b show a bar chart representing the average concentration of trans- and cis-α-irone detected in the reaction catalyzed by pMT3 (Y200F_S182E), pMT3-L2731, pMT3-L273K and pMT3-L273V over replicate reactions. The cis-to-trans-α-irone ratio is shown as a black circle. The L273V mutation gave rise to highest cis-α-irone and lowest trans-α-irone production.

FIG. 13 shows the HS-SPME-GCMS analysis to identify the products in pMT5 and pMT6 reaction. Cell lysates overexpressing IspD (negative control) pMT5, pMT6 were used to assay against psi-ionone. The reaction mixtures were subjected to HS-SPME-GCMS analysis. An additional methylated product that corresponding to β-irone was detected in the reaction containing pMT6. The mass spectrum of cis-α-irone and β-irone are shown. The retention time and mass spectrum of the irones detected are the same as the synthetic chemical standard (std) from Sigma Aldrich.

FIG. 14 shows the data for the Mutagenesis study based on pMT7. FIG. 14a shows the level of cis-α-irone detected in the headspace of reactions catalyzed by ispD (−ve control), pMT7 and pMT7-C156P. FIG. 14b shows the level of cis-α-irone and β-irone detected in the headspace of reactions catalyzed by ispD (−ve control), pMT7 and pMT9 (pMT7-C156A).

FIG. 15 shows the activity and stability analysis of pMT7. FIG. 15a shows the time course of cis-α-irone production by purified pMT7. FIG. 15b shows the time course of cis-α-irone production and psi-ionone consumption by pMT7 in cell extracts. FIG. 15c shows the final amount of cis-α-irone produced by pMT7 in cell extracts when varied amount of cell extracts (OD) or duration of incubation were tested. FIG. 15d shows the native PAGE gel analysis of the oligomeric state of pMT1 and pMT7 enzyme when stored at −20° C., incubated at 28° C. or reacted at 28° C. FIG. 15e shows the final amount of cis-α-irone produced by pMT7 in cell extracts by overnight pre-incubating at 28° C. (28 C) or 4° C. (4 C). FIG. 15f shows the cis-α-irone produced by purified pMT7 when 20 μM SAH or 2.4 μM irone was added into the reaction. All the data shown is an average of replicate reaction.

FIG. 16 show the further mutations to improve pMT7 activity and reduce SAH inhibition. FIG. 16a shows the structural visualization of the hydrogen bond network (dashed line) between SAH and residue D135 and T91 from pMT3 model. A stronger hydrogen bond network is expected between SAH and D135. FIG. 16b shows the level of cis-α-irone detected in the headspace of reactions catalyzed by pMT7, pMT7-D135A, pMT7-D135E, pMT7-D135G and pMT7-D135P. The cis-α-irone levels were significantly reduced when D135 was mutated. FIG. 16c shows the level of cis-α-irone detected in the headspace of reactions catalyzed by pMT7 and pMT7-T91P. The cis-α-irone level was slightly enhanced when T91 was mutated to Proline.

FIG. 17 shows a summary of the mutations from pMT1 to pMT10. It shows the structural visualization of residues mutated from pMT1 to pM10. The mutated residues are shown as spheres and the mutations are labelled. The co-factor SAH is shown in stick representation.

FIG. 18 shows the auxiliary reactions to remove SAH inhibition to pMT reactions. FIG. 18a shows a schematic representation of reactions catalyzed by mtn and mtaD. Mtn hydrolyses SAH into S-ribosyl-L-homocysteine and adenine. mtaD deaminates SAH to S-inosyl-homocysteine and ammonia. FIGS. 18b and 18c show the SDS-PAGE gel analysis of purification of mtn and mtaD respectively. The protein is indicated by the arrow. The lane labels are as follows. M: marker. T: total protein. S: soluble protein. F: flowthrough after his-tag binding. C: concentrated protein by combining all the elution fraction and desalting by ultracentrifugation.

FIG. 19 shows the optimization of in vivo production of psi-ionone and cis-α-irone from a renewable carbon source. FIG. 19a shows a schematic representation of the four plasmids used to optimize psi-ionone production. The first plasmid carried the SAR module, which contained the upper mevalonate enzymes: HMG-COA synthase (HmgS), Acetoacetyl-CoA thiolase (AtoB) and truncated HMG-COA reductase (tHmgR). The second plasmid carried the MPPI module, which contained the lower mevalonate enzymes:

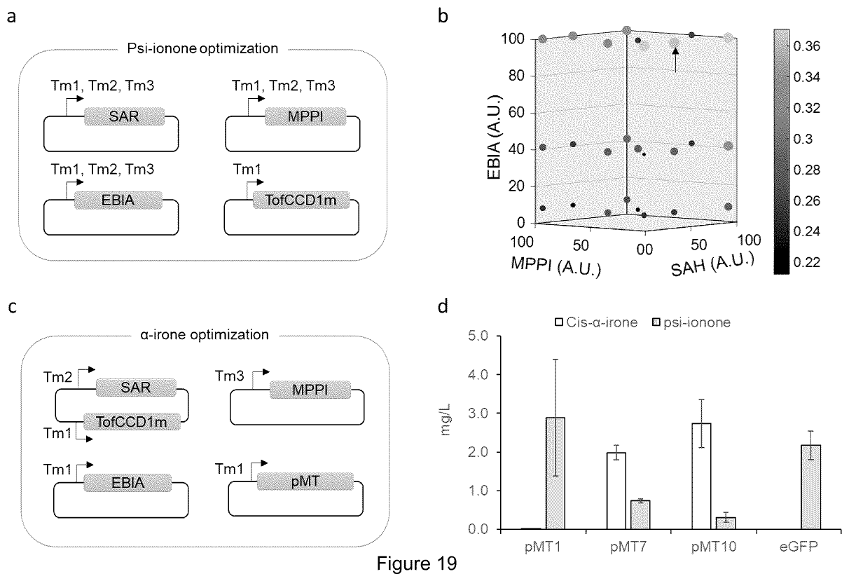

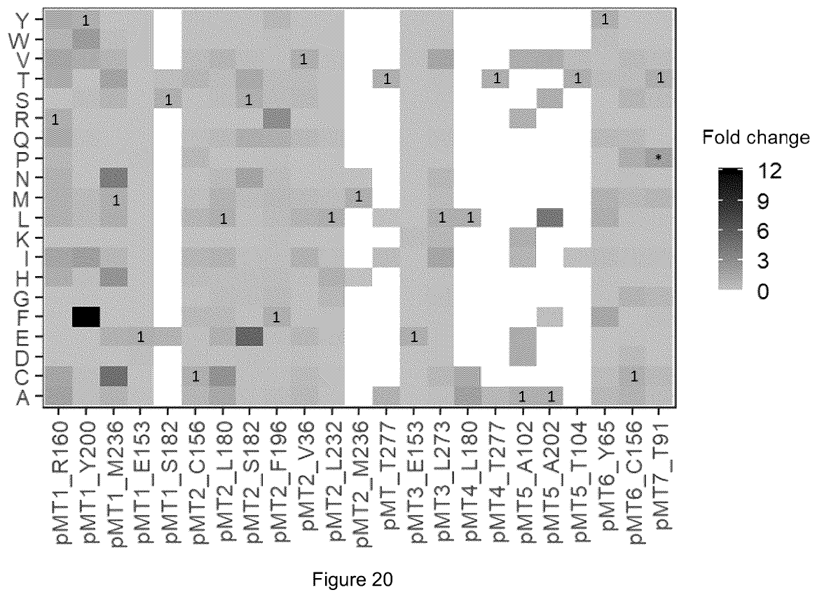

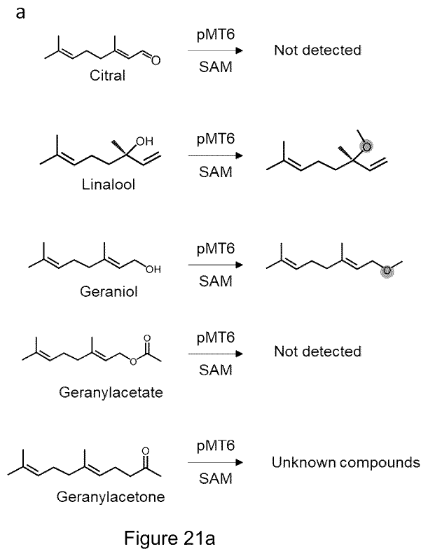

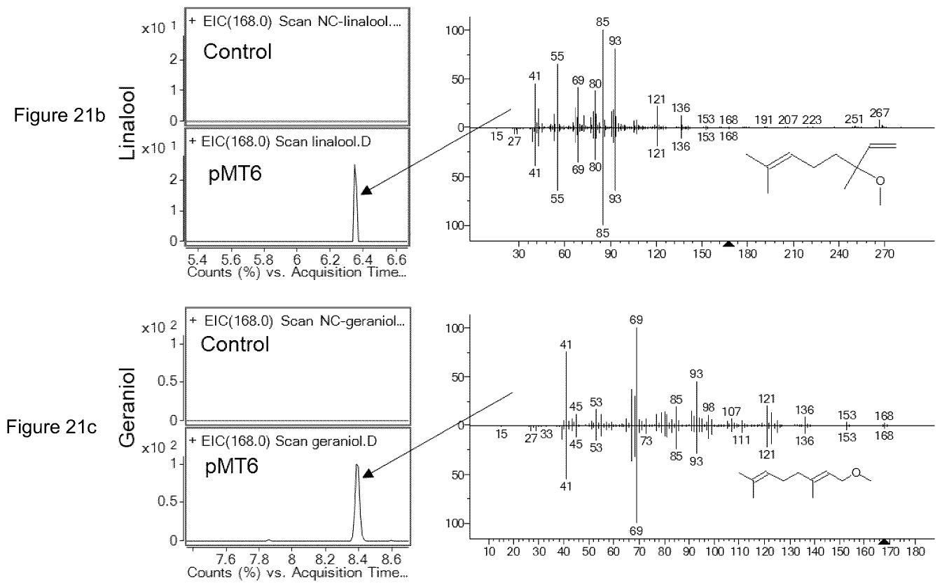

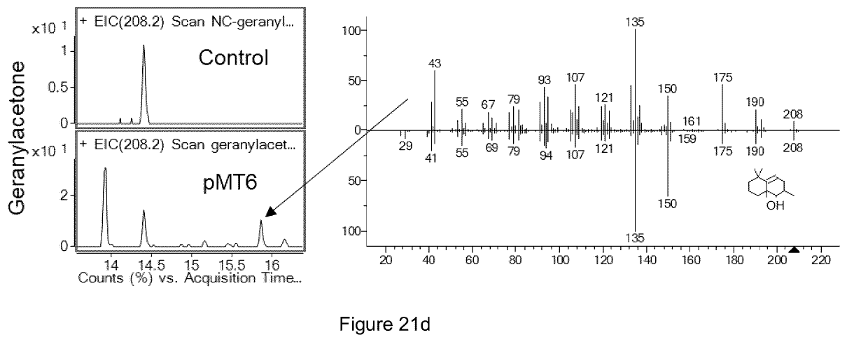

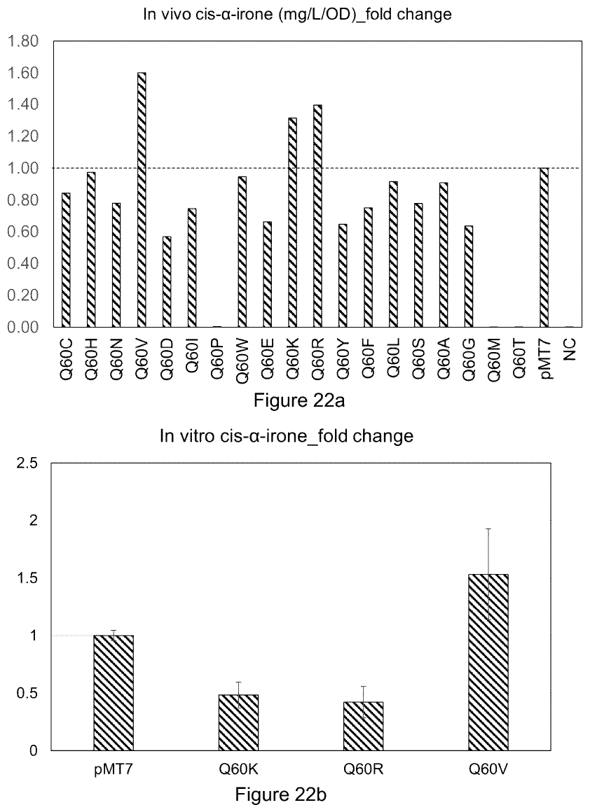

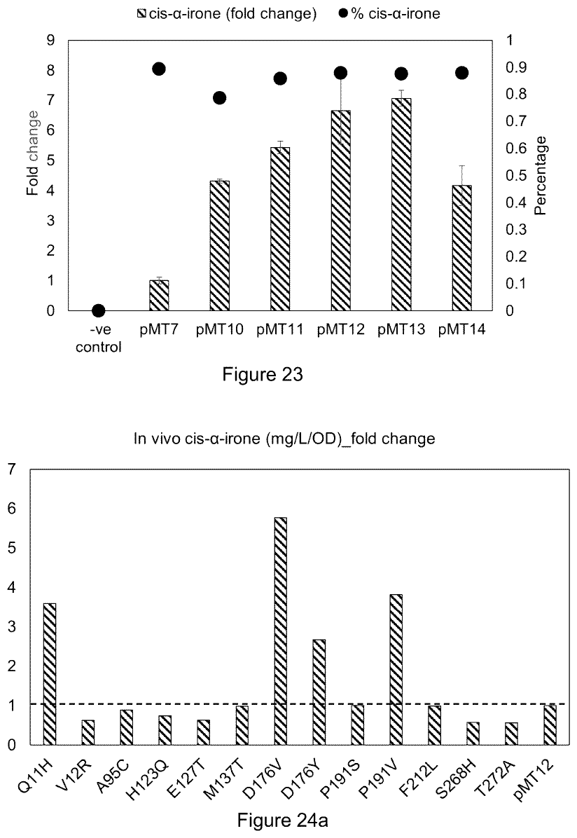

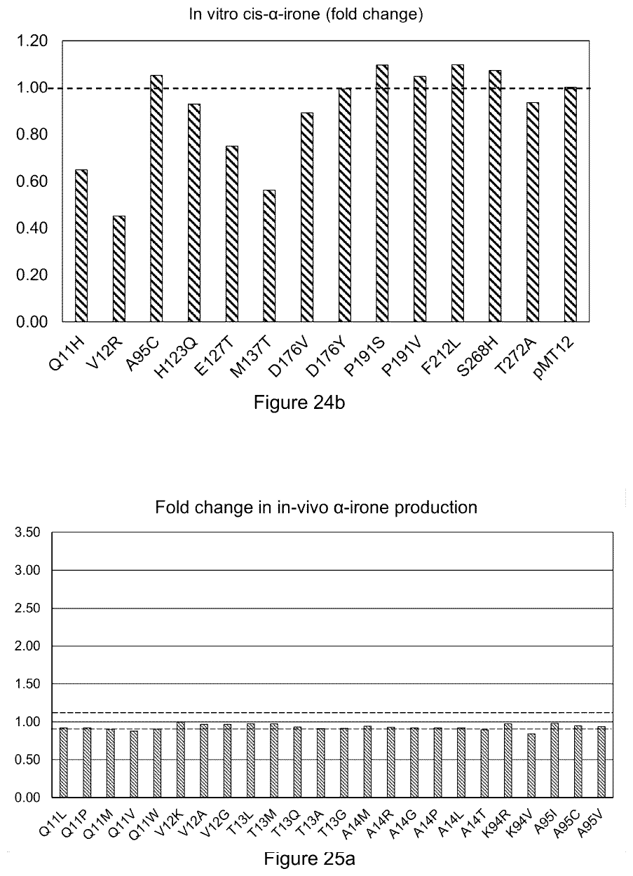

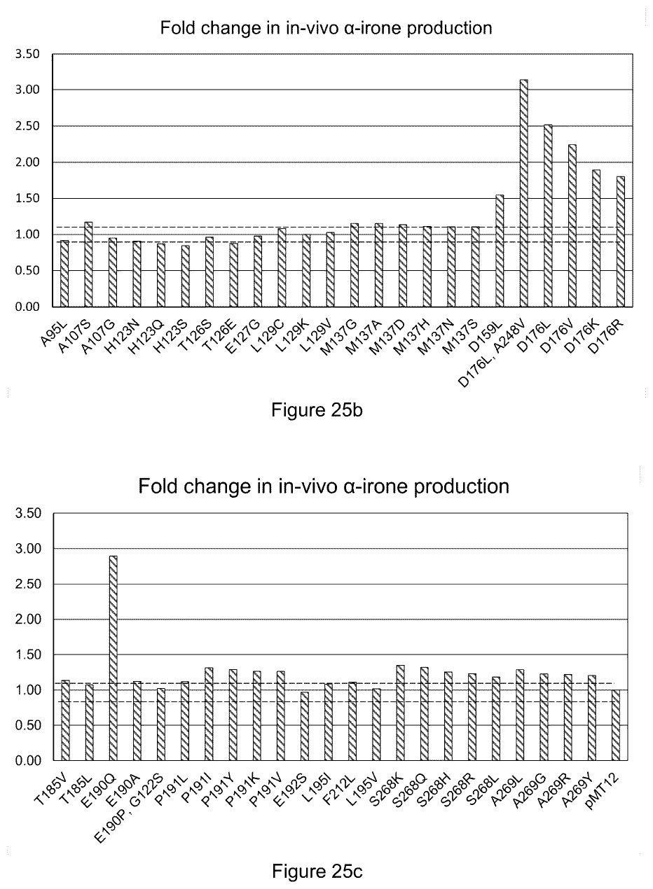

mevalonate kinase (MevK), phosphomevalonate kinase (PMK), mevalonate pyrophosphate decarboxylase (PMD) and IPP isomerase (Idi). The third plasmid carried the lycopene synthesis EBIA module: GGPP synthase (CrtE), phytoene synthase (CrtB), phytoene desaturase (Crtl) and FPP synthase (IspA). The last plasmid carried the modified OfCCD1 enzyme fused with thioredoxin (TofCCD1m)⁷. Tm1, Tm2, Tm3 are mutated T7 promoters with different strength (Tm1>Tm2>Tm3)¹⁵. FIG. 19b shows how MHP was used to optimize psi-ionone specific titer (mg/I/OD) by tuning the promoters controlling the SAR, MPPI and EBIA modules, while keeping the promoter for TofCCD1m as Tm115. The highest specific psi-ionone titer obtained was from strain 2311 (Tm2-SAH, Tm3-MPPI, Tm1-EBIA, Tm1-TofCCD1m) as indicated by the black arrow. To reduce the number of plasmids, the 1st and 4th module were combined into one plasmid and the strain was further named to 2O31. FIG. 19c shows a schematic representation of the four plasmids used to optimize cis-α-irone production in vivo. The first plasmid contained both the SAR module and TofCCD1m. The second plasmid carried the MPPI module. The third plasmid carried the EBIA module. The fourth plasmid carried the pMT enzyme. FIG. 19d shows a bar chart representing the average amount of cis-α-irone and psi-ionone produced over replicated reaction when pMT1, pMT7, pMT10 and eGFP was overexpressed on the fourth plasmid.

FIG. 20 shows a summary of the fold change in cis-α-irone produced by each mutant pMT enzyme in the form of a heatmap. The amount of cis-α-irone produced was estimated based on the area under the gas chromatogram curve (AUC). The fold change is calculated by comparing the AUC of cis-α-irone produced by the mutant enzyme to that of non-mutated which is indicated by 1 in the heatmap. * indicates the mutant pMT10.

FIG. 21 show the methylation and cyclization of selected terpenoids.

FIGS. 22a and 22b show the fold change of cis-α-irone production with Q60 mutants of pMT7 in-vivo and in-vitro respectively.

FIG. 23 shows the fold change and percentage of cis-α-irone with pMT7, pMT10, pMT11, pMT12, pMT13, and pMT14.

FIGS. 24a and 24b show the fold change of cis-α-irone production with mutants of pMT12 in-vivo and in-vitro respectively.

FIGS. 25a, 25b, and 25c show the fold change of cis-α-irone production with mutants of pMT12.

Some of the figures are composed of panels which are labelled alphabetically, the panel may be referred to herein as FIG. Xa where X is the Figure number and “a” is the appropriate panel.

### DETAILED DESCRIPTION

The following references provide one of skill with a general definition of many of the terms used herein: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991).

Various amino acids are described herein by its full name, and conventional 1-letter and 3-letter abbreviations as is known in the art. The substitution of an amino acid residue in a peptide is describe by the conventional notation, for example Y200F indicates that the 200th amino acid residue (or position) of tyrosine (Y) is substituted by phenylalanine (F).

As used herein, the articles “a”, “an” and “the” as used with regard to a feature or element include a reference to one or more of the features or elements. As used herein, the term “and/or” includes any and all combinations of one or more of the associated listed items. As used herein, the terms “first,” “second,” and “third,” etc. are used merely as labels, and are not intended to impose numerical requirements on their objects. In particular, the modified enzyme may comprise any combination of the first substitution, the second substitution, the third substitution and so forth, it is not required that the modified enzyme contains all the substitutions. As an example, the modified enzyme may comprise the first substitution and the third substation without requiring the second substitution, other substitution patterns may also be possible as described herein.

The terms “polypeptide” and “protein”, used interchangeably herein, refer to a polymer of amino acids without regard to the length of the polymer; thus, peptides, oligopeptides, and proteins are included within the definition of polypeptide. This term also does not specify or exclude chemical or post-expression modifications of the polypeptides of the invention, although chemical or post-expression modifications of these polypeptides may be included or excluded as specific embodiments. Therefore, for example, modifications to polypeptides that include the covalent attachment of glycosyl groups, acetyl groups, phosphate groups, lipid groups and the like are expressly encompassed by the term polypeptide. Further, polypeptides with these modifications may be specified as individual species to be included or excluded from the present invention. The natural or other chemical modifications, such as those listed in examples above can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of 14 hosphatidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. [See, for instance Creighton, (1993), Posttranslational Covalent Modification of Proteins, W.H. Freeman and Company, New York B. C. Johnson, Ed., Academic Press, New York 1-12; Seifter, et al., (1990) Meth Enzymol 182:626-646; Rattan et al., (1992) Ann NY Acad Sci 663:48-62]. Also included within the definition are polypeptides which contain one or more analogs of an amino acid (including, for example, non-naturally occurring amino acids, amino acids which only occur naturally in an unrelated biological system, modified amino acids from mammalian systems, etc.), polypeptides with substituted linkages, as well as other modifications known in the art, both naturally occurring and non-naturally occurring.

As used herein, the terms “recombinant polynucleotide” and “polynucleotide construct” are used interchangeably to refer to linear or circular, purified or isolated polynucleotides that have been artificially designed and which comprise at least two nucleotide sequences that are not found as contiguous nucleotide sequences in their initial natural environment. In particular, these terms mean that the polynucleotide or cDNA is adjacent to “backbone” nucleic acid to which it is not adjacent in its natural environment. Additionally, to be “enriched” the cDNAs will represent 5% or more of the number of nucleic acid inserts in a population of nucleic acid backbone molecules. Backbone molecules according to the present invention include nucleic acids such as expression vectors, self-replicating nucleic acids, viruses, integrating nucleic acids, and other vectors or nucleic acids used to maintain or manipulate a nucleic acid insert of interest. Preferably, the enriched cDNAs represent 15% or more of the number of nucleic acid inserts in the population of recombinant backbone molecules. More preferably, the enriched cDNAs represent 50% or more of the number of nucleic acid inserts in the population of recombinant backbone molecules. In a highly preferred embodiment, the enriched cDNAs represent 90% or more (including any number between 90 and 100%, to the thousandth position, e.g., 99.5%) of the number of nucleic acid inserts in the population of recombinant backbone molecules.

The term “recombinant polypeptide” is used herein to refer to polypeptides that have been artificially designed and which comprise at least two polypeptide sequences that are not found as contiguous polypeptide sequences in their initial natural environment, or to refer to polypeptides which have been expressed from a recombinant polynucleotide.

The terms “sequence similarity”, “percentage of sequence identity” and “percentage homology” are used interchangeably herein to refer to comparisons among polynucleotides and polypeptides, and are determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Identity is evaluated using any of the variety of sequence comparison algorithms and programs known in the art. Such algorithms and programs include, but are by no means limited to, TBLASTN, BLASTP, FASTA, TFASTA, CLUSTAL W, FASTDB [Pearson and Lipman, (1988), Proc. Natl. Acad. Sci. USA 85(8):2444-2448; Altschul et al., (1990), J. Mol. Biol. 215(3):403-410; Thompson et al. (1994), Nucleic Acids Res. 22(2):4673-4680; Higgins et al., (1996), Meth. Enzymol. 266:383-402; Altschul et al., (1993), Nature Genetics 3:266-272; Brutlag et al. (1990) Comp. App. Biosci. 6:237-24], the disclosures of which are incorporated by reference in their entireties.

Metabolic engineering has become an attractive method for efficient production of natural products. However, one important pre-requisite is to establish the biosynthetic pathways. Many commercially interesting molecules cannot be biosynthesized as their native biochemical pathways are not fully elucidated. Cis-α-irone, a top-end perfumery molecule, is an example. Retrobiosynthetic pathway design by employing promiscuous enzymes provides a solution. Described herein is a synthetic pathway to produce cis-α-irone with a promiscuous methyltransferase (pMT). By structure-guided enzyme engineering strategies, pMT activity and specificity towards cis-α-irone was improved by greater than 11,000-fold and 700-fold respectively. By incorporating the optimized methyltransferase into engineered microbial cells, approximately 172 mg/L of cis-α-irone was produced from a renewable carbon source in a 5 L bioreactor. This illustrates that integrated retrobiosynthetic pathway design and enzyme engineering can offer novel opportunities to expand the scope of natural molecules that can be biosynthesized.

A total enzymatic synthetic route to produce cis-α-irone from glucose has been developed by identifying and optimizing a promiscuous bifunctional methyltransferase/cyclase enzyme (pMT, FIG. 1). With structure-guided directed evolution, we have improved the activity and selectivity of pMT towards cis-α-irone by greater than 11,000-fold and 700-fold respectively. About 182 mg/L cis-α-irone was produced by one-step biotransformation of synthetic psi-ionone. By incorporating the engineered pMT enzyme in the psi-ionone producing microbe, approximately 172 mg/L cis-α-irone production from glucose was achieved in a fed-batch process. This provides a plant-independent route to biosynthesize enantiopure cis-α-irone. The integrated synthetic pathway design and rational enzyme engineering approach is promising to produce natural products whose native biosynthetic pathways are yet to be discovered.

### Synthetic Pathway Design and Validation

FIG. 1 shows the schematic representations of the different pathways to produce irone. The chemical route uses an acid to catalyze cyclisation of methyl-3-psi-ionone, which is not a known natural product. Chemical synthesis produces a mixture of stereoisomers including (1R,5R)-trans-α-irone (1R5R), (1S,5S)-trans-α-irone (1S5S), (1S,5R)-cis-α-irone (1S5R), (1R,5S)-cis-α-irone (1R5S), (5R)-β-irone and (5S)-β-irone. The native biosynthesis route was proposed based on radiolabeling study⁶, and a bifunctional methyltransferase and cyclase (bMTC) was hypothesized to convert iridal directly to cycloiridal. Cycloiridal is asymmetric, so only one molecule of α-irone is produced from the C30 precursor. The resulting carbon yield is 46.7%.

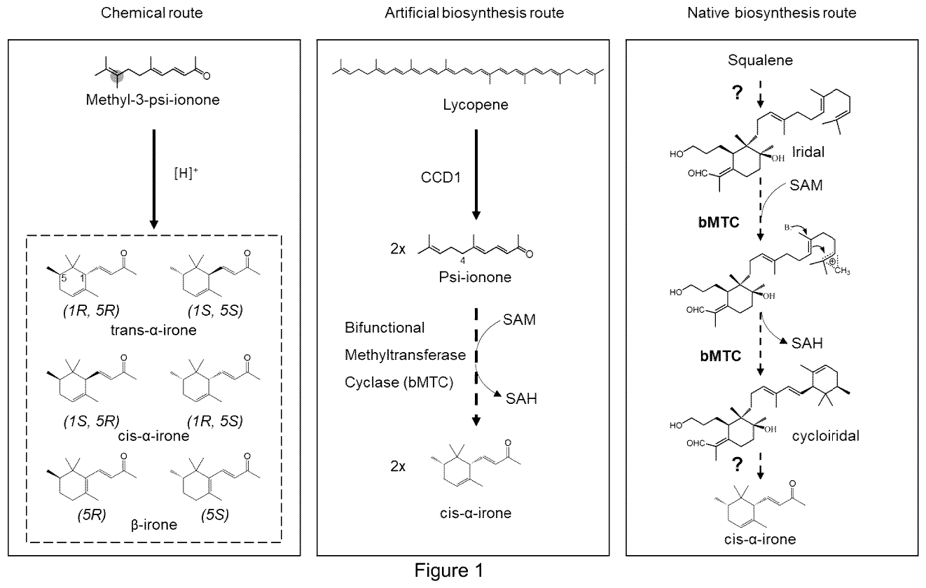

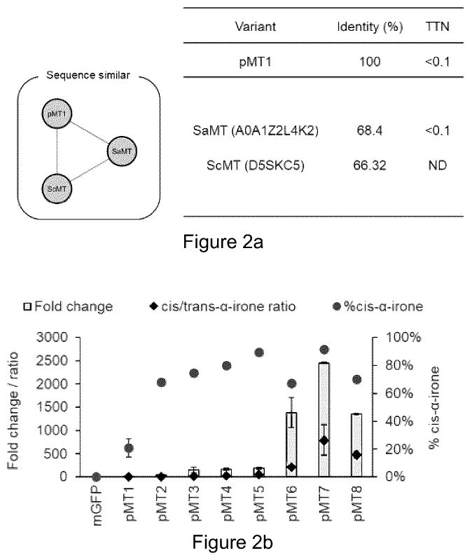

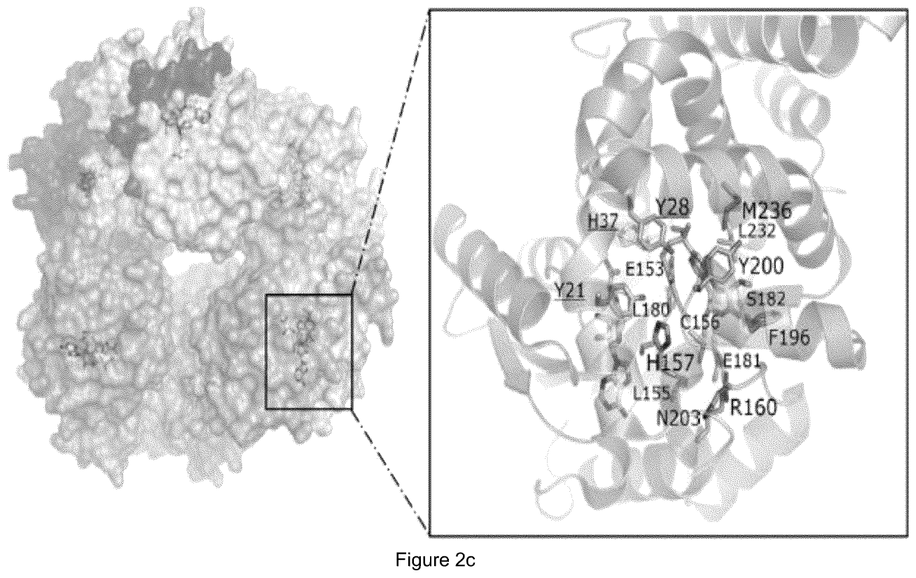

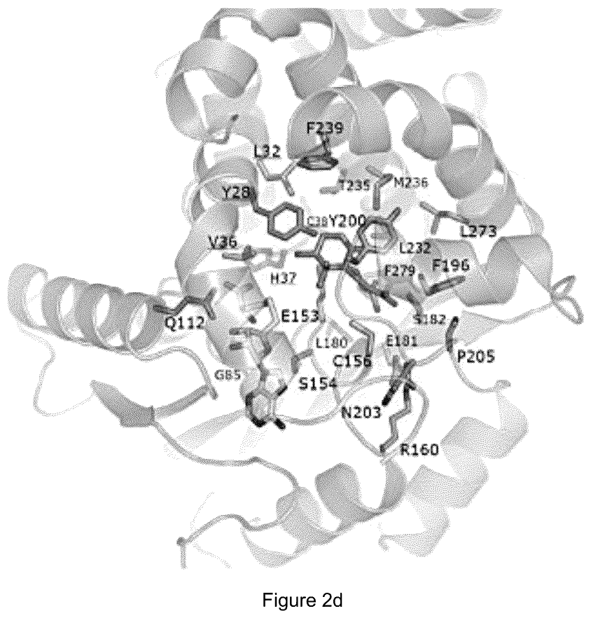

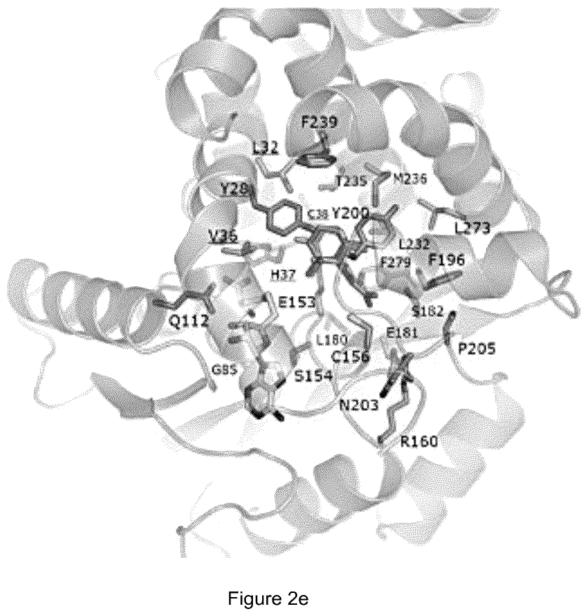

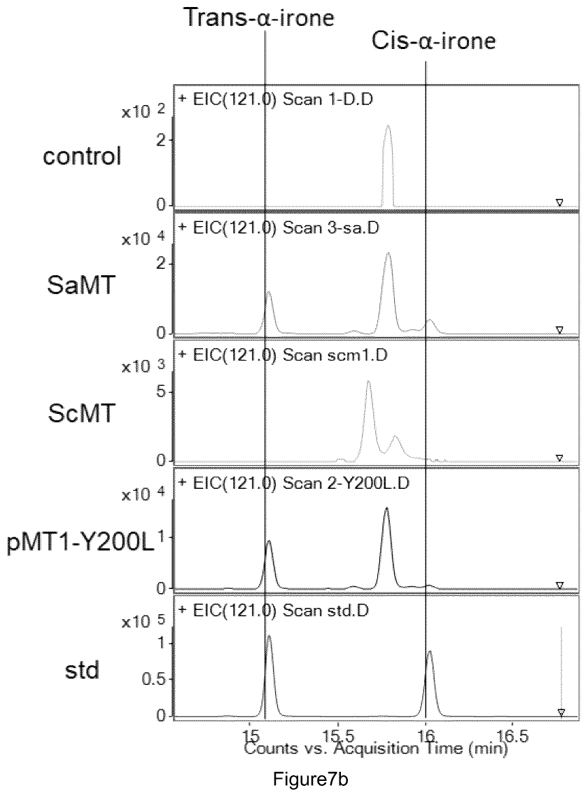

Inferring from knowledge of chemical and biochemical synthesis, it was postulated that psi-ionone can be converted to irone by a bMTC enzyme (artificial biosynthesis route, FIG. 1). The artificial biosynthesis route was designed by combining both the chemical and biological synthesis knowledge, namely, to use a promiscuous bMTC to convert psi-ionone into α-irone. Two molecules of psi-ionone molecules are readily formed from the symmetric C40 lycopene by carotenoid cleavage dioxygenase (CCD1)^{7,8}. Hence, two molecules of α-irone are expected to be formed from the C40 precursor whereas only one molecule of irone can be formed from asymmetric iridal (FIG. 1). Theoretically, the artificial route has a higher carbon yield than that for the native biosynthetic route from their respective terpenoid precursors (65% vs 46.7%, FIG. 1). Serendipitously, a bifunctional methyltransferase and cyclase (TleD or pMT1, SEQ ID No. 3, in this study) was recently discovered from Streptomyces9 and structurally elucidated (PDB id: 5GM1 (substrate-free), 5GM2 (substrate-bound)) 10. By incubating cell extracts overexpressing pMT1 with psi-ionone, both trans-α-irones (79%) and cis-α-irones (21%) were detected (FIGS. 2a, 2b, and 7).

To identify a potentially more active enzyme, pMT1 sequence (SEQ ID No. 3) was mined using BLAST against the non-redundant protein sequence database11 and two sequences were identified to have greater than 60% sequence identity to pMT1 (FIG. 2a). Both enzymes were recombinantly produced in E. coli and tested. Only methyltransferase from Streptomyces albireticuli (SaMT, SEQ ID No. 1, about 68% sequence identity to pMT1) converted psi-ionone to predominantly trans-α-irone. The other about 66% homologous enzyme from Streptomyces clavuligerus (ScMT, SEQ ID No. 2) was not active towards psi-ionone (FIG. 2a and FIG. 7). Based on the crystallographic structure of pMT1, pMT1 was modified to further improve its catalytic efficiency.

### Structure-Based Enzyme Engineering to Improve pMT Activity

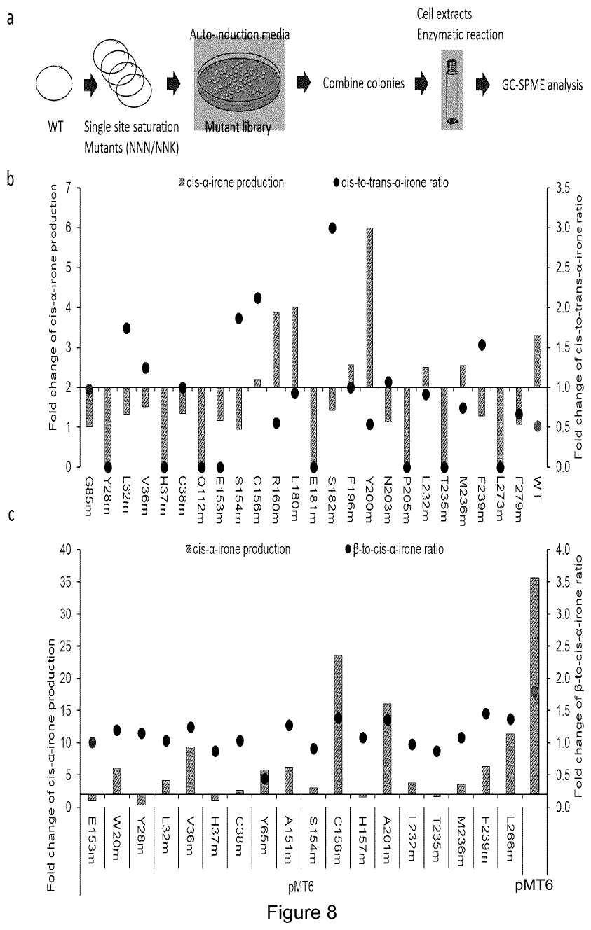

In solution and in X-ray structure, pMT1 shows a hexameric assembly whose minimal functional unit is a dimer10. pMT1 displays a unique active site formed by amino acid residues from two distinct monomeric chains of the hexamer, with the N-terminus of one monomer covering the catalytic cavity of the other monomer (FIG. 2c). Using the structure of pMT1 in complex with the cofactor S-adenosyl-L-homocysteine (SAH) and the substrate teleocidin A1 (PDB id: 5GM2) as a template, pMT1 bound to each of the four isomers of α-irone was modelled with the aim of identifying amino acid residues that could be critical to increase cis-α-irone or decrease trans-α-irone production (FIGS. 2d and 2e). Visual inspection of the pMT1: α-irone complexes enabled identification of 24 amino acid residues lining the substrate- and cofactor-binding pocket. Site-directed mutagenesis of the 24 residues was carried out with degenerative primers. Subsequently, the mutants of each site were pooled together, and examined if the activity or product-selectivity of the pooled mutants were increased (FIG. 8). This reduced the number of screening reactions to 24, which is manageable when a high-throughput enzymatic assay is not available. FIG. 8a shows the schematic representation of the pooled-colony reactions. One residue was chosen and subjected to site-saturation mutagenesis by using degenerative primers containing NNN or NNK. The plasmid library was then transformed into E. coli BL21 (DE3) cells and plated on agar plate containing autoinduction media¹⁹. Optimization was carried out to ensure there were more than 200 colonies formed on the agar plates to achieve a good coverage of the mutants. Subsequently, the colonies were collected and resuspended in phosphate saline buffer. Equal number of cells was taken for lysis and cell lysate reactions were carried out in capped SPME-GC vials at 28° C. for 2 days. HS-SPME-GCMS analysis was used to quantify the amount of irones produced by each pooled-colony reaction. FIG. 8b shows the pooled-colony reactions to identify the beneficial active site residue(s) to mutate to improve pMT1 activity and selectivity towards cis-α-irone. 24 residues including the control Gly85 (G85m) were subjected to site-saturation mutagenesis, using pMT1 as the template. Table 7 and FIG. 20 shows a summary of the fold change in cis-α-irone produced by each mutant pMT enzyme. The amount of cis-α-irone produced was estimated based on the area under the gas chromatogram curve (AUC). The fold change is calculated by comparing the AUC of cis-α-irone produced by the mutant enzyme to that of non-mutated.

Among them, site-saturated mutants of G85 were also included. This is a key residue involved in SAM co-factor binding. Mutating this glycine to the other 19 amino acids significantly reduces the methyltransferase activity. Hence, the activity of the pooled G85 mutants could be an approximate baseline to access the impact of mutation on the activity of pMT: if the pool mutants have lower or similar activities as G85 mutants, the corresponding residues are probably essential for methyltransferase activity and should not be mutated; otherwise, the corresponding residues are potential targets for further analysis to identify the specific beneficial mutation.

The 24 mutant libraries and 1 non-mutated wild-type (WT) pMT1 were transformed into E. coli for pooled-colony reaction. Trans-α-irone and cis-α-irone produced from each pooled mutant library were quantified. The fold change was calculated by dividing the cis-α-irone produced from one reaction with cis-α-irone produced by the G85m reaction. Similarly, cis-α-irone to trans-α-irone ratio (cis-to-trans-α-irone ratio) was also quantified and the fold change was calculated against the G85m control.

Among the 24 pooled reactions, seven positions displayed at least two-fold improvement in cis-α-irone production as compared to the baseline activity (i.e. G85 mutation), and three out of the seven residues, namely Y200, L180 and R160, gave rise to higher cis-α-irone production as compared to pMT1 (FIG. 8). Moreover, more than half of the mutated residues showed altered product selectivity, increasing the cis-to-trans-α-irone ratio as compared to pMT1. Among them, S182 was the most promising residue to be mutated, which showed the highest cis-to-trans-α-irone ratio (FIG. 8).

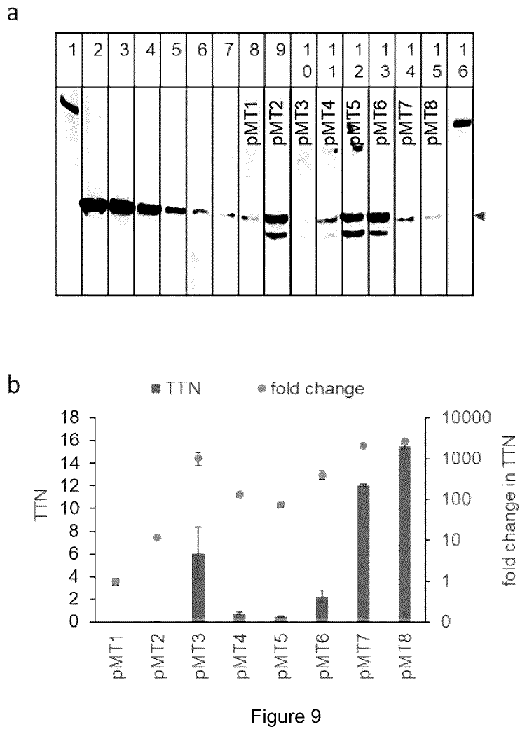

Subsequently, site-saturated mutagenesis of Y200 was carried out. A Y200F mutation (pMT2, SEQ ID No. 4), improved the soluble expression of the methyltransferase and its total turnover number (TTN) by greater than 3-fold and 12-fold respectively (FIGS. 9, 20 and Table 7). More importantly, the cis-isomer content increased to ˜68% (FIG. 2b). It is interesting to note that a Y200L mutation, a naturally occurring residue in SaMT, improved trans-α-irone production (FIG. 7b), suggesting that Y200 plays a role in controlling the stereoselectivity of the products.

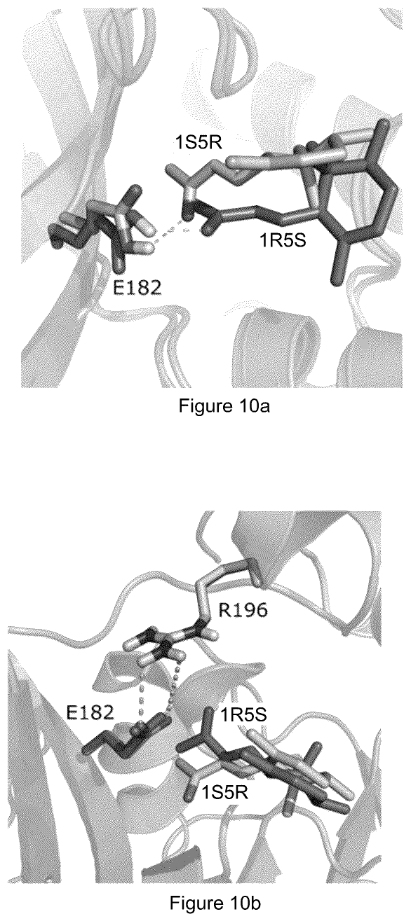

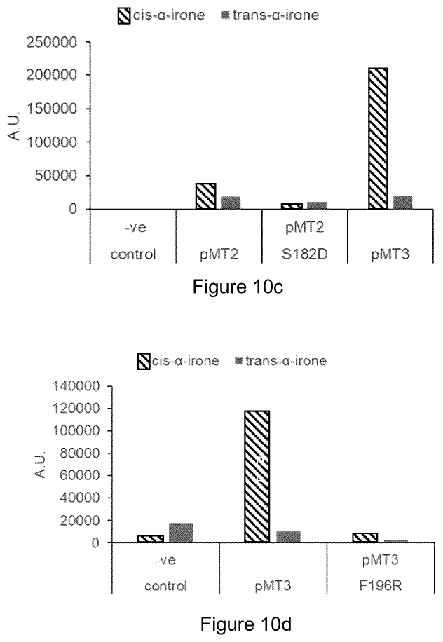

Next, site-saturation mutagenesis was performed on L180 and S182 using pMT2 as the new template (Table 7 and FIG. 20). A L180 mutation to alanine resulted in a marginal increase in cis-α-irone production. Remarkably, one mutant, S182E (pMT3, SEQ ID No. 5), significantly improved TTN by more than 1,000-fold as compared to pMT1 (FIG. 9b) and increased the cis-α-irone content to 74% (FIG. 2b). Structure and predicted pKa analysis revealed that E182 (predicted pKa=7.2) is mostly protonated under neutral pH, thus it may establish a polar contact with the ketone moiety of psi-ionone or irone. This polar interaction could be important for enzymatic activity. Indeed, mutating S182 to aspartate did not elicit the same enzymatic activity, as the distance between the carboxylate side chain and the ketone moiety probably became too long for any polar contact. Moreover, when the adjacent F196 was further mutated to arginine, creating a salt bridge between E182 and R196, the methyltransferase activity towards psi-ionone was nearly abolished (FIG. 10b).

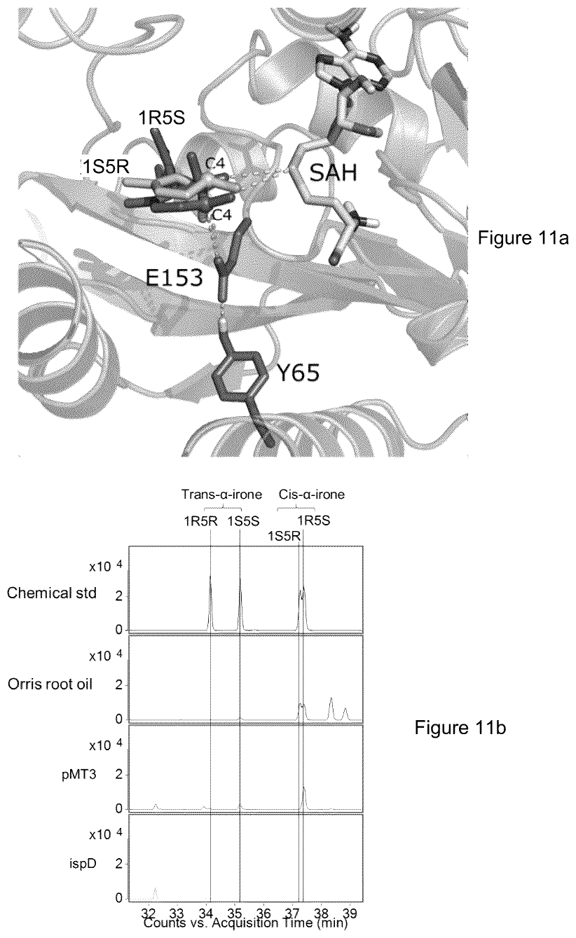

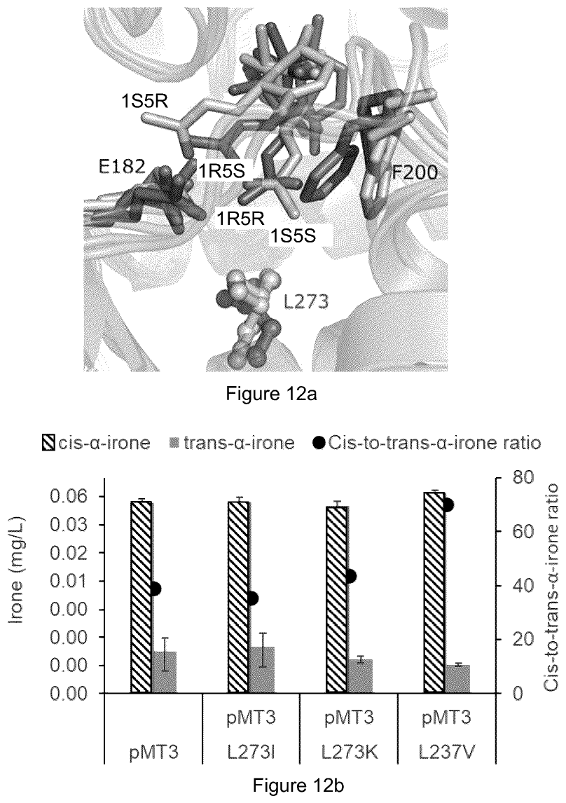

To further improve the enzymatic activity and selectivity of the promising mutant (pMT3), molecular dynamics simulations were carried out using the modelled complexes of the four isomers of α-irone and pMT1 (or pMT3) to identify amino acid residues that could be critical to improve binding of cis-α-irone or decrease binding of trans-α-irone. Visual inspection and free energy calculations were performed. Interestingly, free energy calculations showed a significant change of the free energy profile of an important residue, E153 (Table 1). Table 1 shows the free energy contributions per residue computed from MM/GBSA approaches. The free energy calculations were performed using MD simulations from pMT1/3/4 in complex with each of the four isomers of α-irone. The energy profile change of the catalytic residue E153 is highlighted by showing the unfavorable contributions in bold. pMT1 corresponds to the wild-type whereas pMT3 is the Y200F/S182E mutant and pMT4 the Y200/S182E/L273V mutant. The numbering of the residues corresponds to the X-ray structure (PDB id: 5GM2). The free energy values are given in kcal/mol and correspond to the mean from 100 snapshots extracted uniformly. Indeed, based on the proposed mechanism and crystal structure of pMT110, E153 is the key catalytic residue that extracts the proton from psi-ionone (FIG. 11a). The free energy contribution of E153 appears improved in pMT3 compared to pMT1 to favor binding to cis-isomers, mainly (1R, 5S)-cis-α-irone (1R5S), in accordance with results shown in FIG. 2b. Chiral GC analysis showed that predominantly 1R5S was produced by pMT3 reaction (FIG. 11b). Free energy calculations also confirmed the favorable contributions of the two mutated residues in pMT3, i.e. Y200F and S182E (Table 1). For all MD simulations with pMT3, Y200F and S182E formed an energetically and structurally favorable environment to interact with α-irone. This environment was structurally maintained by L273 which was proposed for the next round of mutation (FIG. 12a). Mutating L273 to lysine or valine revealed further improvement of the enzymatic selectivity; L273V (pMT4, SEQ ID No. 6) improved cis-α-irone content to ˜80% by reducing trans-α-irone production (FIG. 2b and FIG. 12b). These results agree with computational predictions for pMT4, that showed an increase of the free energy contribution of E153 toward trans-isomers compared to pMT1 and pMT3 mutants (Table 1). Combining with L180A (pMT5, SEQ ID No. 7), the enzyme expression was improved, and the cis-α-irone content was further increased to ˜90% (FIG. 9 and FIG. 2b).

TABLE 1
Free energy contributions per residue
computed from MM/GBSA approaches

| | E153 | | | Y200 | | |
|---|---|---|---|---|---|---|
| | pMT1 | pMT3 | pMT4 | pMT1 | pMT3 | pMT4 |
| cis/1R5S | 1.14 | −0.36 | −0.10 | −1.82 | −2.14 | −1.63 |
| cis/1S5R | −0.09 | −0.18 | −0.60 | −1.29 | −0.90 | −0.95 |
| trans/1R5R | −0.20 | −0.16 | 0.11 | −1.45 | −1.44 | −1.45 |
| trans/1S5S | −0.03 | 0.00 | 0.70 | −2.07 | −2.14 | −2.12 |
| | S182 | | | L273 | | |
| | pMT1 | pMT3 | pMT4 | pMT1 | pMT3 | pMT4 |
| cis/1R5S | −0.19 | −0.88 | −0.22 | −0.09 | −0.05 | −0.53 |
| cis/1S5R | −0.79 | −1.01 | −0.39 | −0.29 | −0.23 | −0.14 |
| trans/1R5R | −0.38 | −0.90 | −1.37 | −0.23 | −0.10 | −0.07 |
| trans/1S5S | −0.30 | −1.40 | −1.88 | −0.09 | −0.33 | −0.34 |

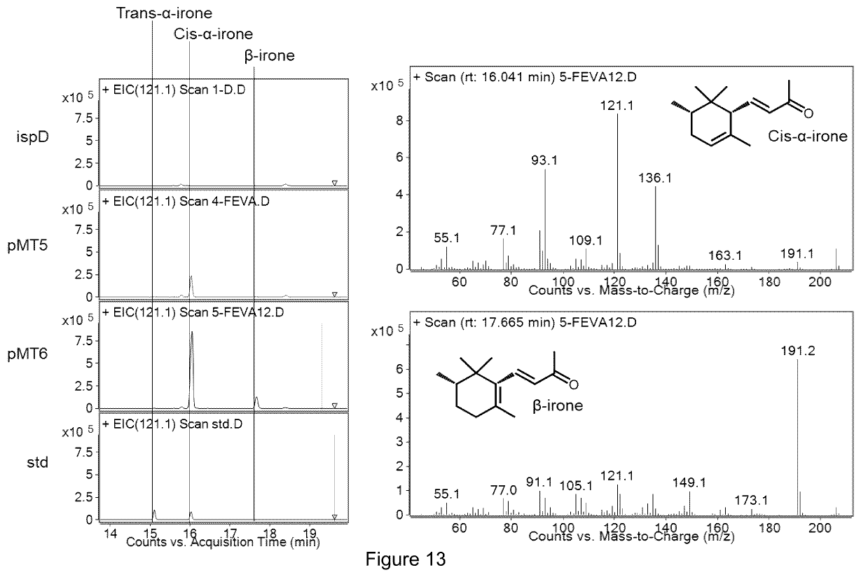

Moreover, since psi-ionone (556.3 Å2) is smaller than pMT1's natural substrate (teleocidin A1, 1129.2 Å²), A202 was mutated to bulkier amino acids (leucine, valine or phenylalanine) based on pMT5 to shrink the binding pocket, in order to increase the affinity of the enzyme for the substrate. Among the new mutants, A202L (pMT6, SEQ ID No. 8) significantly increased the cis-α-irone concentration (FIG. 2b). At the same time, we observed an additional peak corresponding to β-irone (˜30% of the irone mixture, FIG. 13). This led us to examine any mutation(s) that would shift the enzymatic selectivity from β-irone to cis-α-irone.

Due to the catalytic role of E153, the mutation of surrounding residues is expected to influence the enzyme selectivity. 18 amino acid residues were identified in the close vicinity of E153. Pooled experiments were carried out again using pMT6 as template and a E153 mutation was included as baseline activity (FIG. 8c). FIG. 8c shows the pooled-colony reactions to identify the beneficial residue(s) to mutate to improve pMT6 selectivity towards cis-α-irone. 18 residues including the control Glu153 (E153m) were subjected to site saturation mutagenesis using pMT6 as template. 18 mutant libraries and pMT6 were transformed into E. coli for pooled-colony reaction. Cis-α-irone and β-irone were quantified. The fold change was calculated by dividing the cis-α-irone or β-irone produced from one reaction with cis-α-irone or β-irone produced by the E153m reaction respectively. Similarly, β-irone to cis-α-irone ratio (β-to-cis-a-irone ratio) was also quantified.

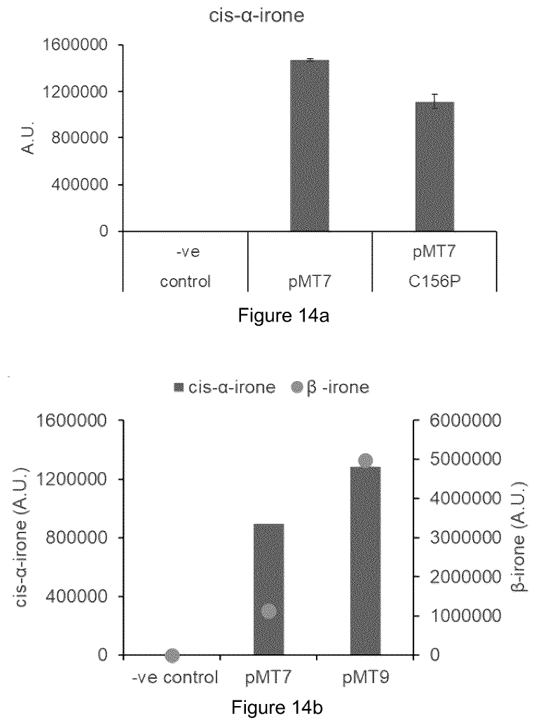

Among them, mutations of Y65 showed a drastic decrease in the β-irone production, and C156 mutations displayed the highest amount of cis-α-irone produced (FIG. 8). Site-saturation mutagenesis of Y65 led to the discovery of Y65F (pMT7, SEQ ID No. 9) that significantly increased the product selectivity towards cis-α-irone (FIG. 2b, FIG. 20 and Table 7). The cis-α-irone production and TTN of pMT7 were 2,450-fold and 2,000-fold higher as compared to pMT1, respectively (FIG. 2b and FIG. 9). The cis-α-irone content was increased to 91.4% (FIG. 2b). Structurally, this mutation removed the hydrogen bond between Y65 and E153, which enabled the catalytic residue to be closer to the C4 hydrogen of psi-ionone (FIG. 11a). We also tested site-saturation mutagenesis on C156 residue based on pMT6, and C156P (pMT8, SEQ ID No. 10) further improved the TTN of the enzyme (FIG. 9, FIG. 20 and Table 7). However, it did not improve the product selectivity, and exacerbated the protein expression (FIG. 2b and FIG. 9). Mutating C156 to proline based on pMT7 resulted in lower methyltransferase activity (FIG. 14a). Instead, C156A mutation (pMT9, SEQ ID No. 11) based on pMT7 increased both α- and β-irone production (FIG. 14b). Since pMT7 was active and selective towards cis-α-irone, pMT7 was further characterized in vitro.

Q60 was identified as another possible mutation site. Site-saturation mutagenesis of Q60 was performed and the Q60V (SEQ ID No. 21), Q60K (SEQ ID No. 22), and Q60R (SEQ ID No. 23) mutants were found to have increased cis-α-irone production in-vivo (FIG. 22a), but of these three mutants only Q60V increased the cis-α-irone production in-vitro (FIG. 22b).

Table 2 below shows the active site residues (or residues in or near the binding pocket) of pMT1, SaMT, and ScMT. The residues in pMT1 that have been mutated and tested to determine if there is improved activity is indicated in bold. Active site residues in pMT7 which are different from SaMT and ScMT are underlined. In addition, some beneficial mutations may not be found within the binding pocket of pMT1, for example Y65F.

TABLE 2
Comparison of active site residues of pMT1, SaMT, and ScMT.
Positions and residues in pMT1 that have been mutated and
tested to determine if a substitution improves the activity
of pMT1 are indicated in bold. Positions and residues in SaMT
and ScMT that are different from pMT7 are underlined.

| | | |
|---|---|---|
| pMT1 | SaMT | ScMT |

| position | residue | position | residue | position | residue |
|---|---|---|---|---|---|
| 14 | A | 13 | A | 13 | A |
| 17 | V | 16 | V | 16 | V |
| 21 | Y | 20 | Y | 21 | Y |
| 28 | Y | 27 | Y | 27 | Y |
| 32 | L | 31 | L | 31 | L |
| 35 | S | 34 | S | 34 | S |
| 36 | V | 35 | V | 35 | I |
| 37 | H | 36 | H | 36 | H |
| 38 | C | 37 | C | 37 | C |
| 83 | D | 82 | D | 82 | D |
| 85 | G | 84 | G | 84 | G |
| 86 | C | 85 | C | 85 | C |
| 87 | G | 86 | G | 86 | G |
| 91 | T | 90 | S | 90 | S |
| 106 | V | 105 | V | 105 | V |
| 107 | A | 106 | A | 106 | A |
| 108 | V | 107 | I | 107 | I |
| 109 | S | 108 | S | 108 | S |
| 112 | Q | 111 | Q | 111 | Q |
| 134 | A | 133 | A | 133 | A |
| 135 | D | 134 | D | 134 | D |
| 136 | A | 135 | A | 135 | A |
| 137 | M | 136 | Q | 136 | M |
| 152 | I | 151 | V | 151 | I |
| 153 | E | 152 | E | 152 | E |
| 154 | S | 153 | S | 153 | S |
| 155 | L | 154 | I | 154 | L |
| 156 | C | 155 | C | 155 | C |
| 157 | H | 156 | H | 156 | H |
| 158 | M | 157 | M | 157 | M |
| 160 | R | 159 | R | 159 | R |
| 180 | L | 179 | L | 179 | L |
| 181 | E | 180 | E | 180 | E |
| 182 | S | 181 | S | 181 | S |
| 196 | F | 195 | F | 195 | L |
| 200 | Y | 199 | L | 199 | L |
| 202 | A | 201 | S | 201 | A |
| 203 | N | 202 | N | 202 | N |
| 205 | P | 204 | P | 204 | P |
| 232 | L | 231 | L | 231 | L |
| 235 | T | 234 | T | 234 | T |
| 236 | M | 235 | M | 235 | M |
| 239 | F | 238 | M | 238 | L |
| 273 | L | 272 | V | 272 | T |
| 277 | T | 276 | T | 276 | T |
| 279 | F | 278 | F | 278 | F |

### In Vitro Biotransformation of Psi-Ionone to Cis-α-Irone

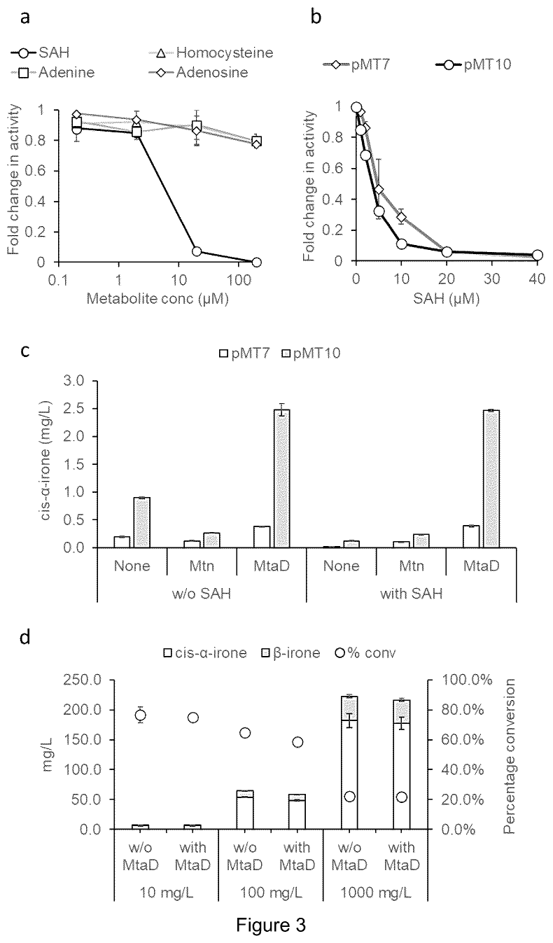

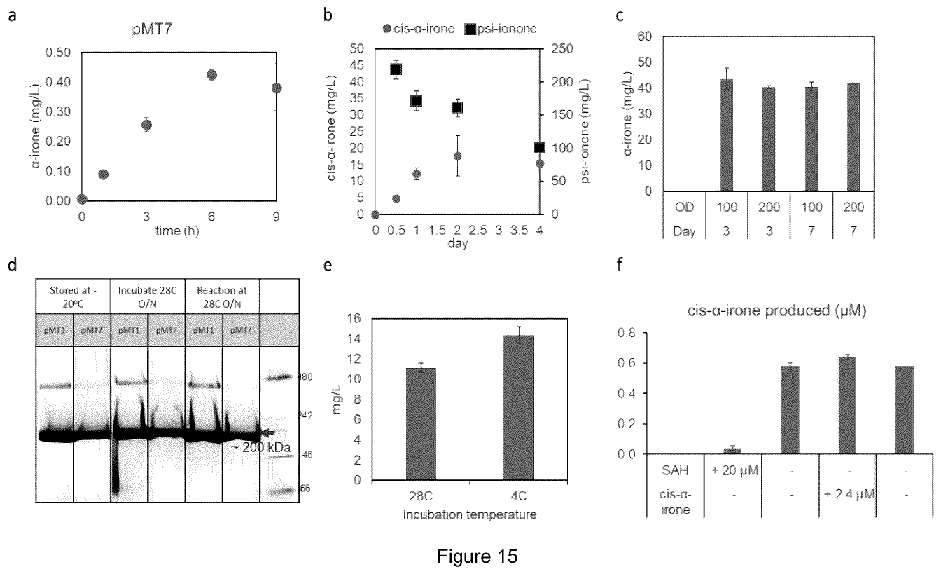

The steady-state kinetic parameters of pMT7 were determined (Table 3). The k_{cat} and Kₘ values for catalyzing psi-ionone were 0.0044 h⁻¹ and 35.2 μM, while those for SAM were 0.0052 h⁻¹ and 54.6 μM. While determining the steady-state kinetics of pMT7, it was noticed that the reaction with purified pMT7 stopped at 6 h before significant conversion had taken place (FIG. 15a). Moreover, while using cell extracts containing pMT7, the reaction reached a plateau after 1-day incubation (FIG. 15b). Increasing the amount of lysates or prolonging the incubation time did not result in any increase in irone production (FIG. 15c). Since pMT7 is an hexamer in solution¹⁰, its oligomeric state before and after the reaction was examined. As shown by the native PAGE gel, pMT7 remained intact as hexamer after incubating or reacting at 28° C. overnight (FIG. 15d). Activity was slightly reduced when the enzyme was pre-incubated at 28° C. (28 C) overnight as compared to 4° C. (4 C) overnight (FIG. 15e), suggesting pMT7 was stable under the reaction temperature. It was further tested if the enzyme was subject to feedback inhibition, as the cofactor product SAH is a potent inhibitor of methyltransferases¹². Supplementing 2 μM of SAH led to a reduction in pMT7 activity and 20 UM of SAH nearly abolished the methyltransferase activity (FIGS. 3a and b). IC₅₀ of SAH for pMT7 was 4.9 μM (Table 3). It was also tested supplementing with SAH analogues (homocysteine, adenine, adenosine) and excess of α-irone, but these did not alter the methyltransferase activity significantly (FIG. 3a and FIG. 15f).

TABLE 3
Kinetics parameters of pMT7 and pMT10.

| | | Kₘ | k_{cat} | k_{cat}/Kₘ | IC₅₀ SAH |
|---|---|---|---|---|---|
| | Substrate | (μM) | (×10³ h⁻¹) | (M⁻¹s⁻¹) | (μM) |
| pMT7 | Psi-ionone | 35.2 ± 4.9 | 4.4 ± 0.2 | 0.035 | |
| | SAM | 54.6 ± 0.1 | 5.2 ± 0.02 | 0.026 | 4.9 |
| pMT10 | Psi-ionone | 26.1 ± 0.4 | 39.8 ± 0.2 | 0.42 | |
| | SAM | 30.5 ± 1.3 | 51.4 ± 0.02 | 0.47 | 3.1 |

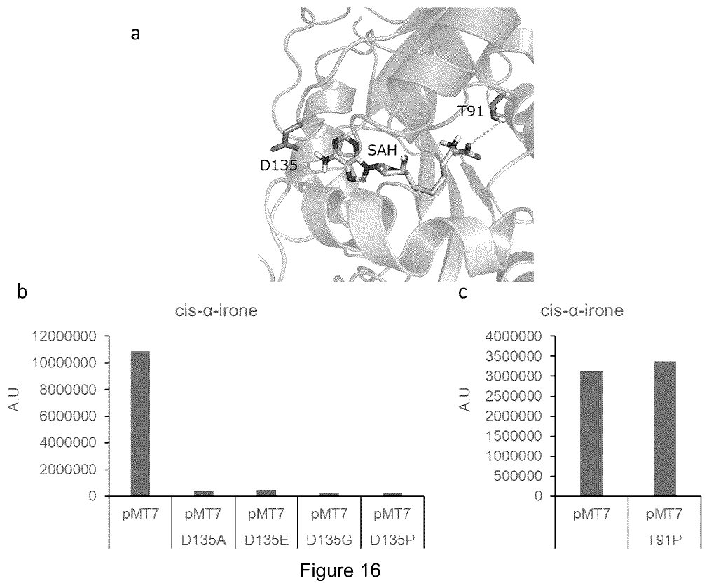

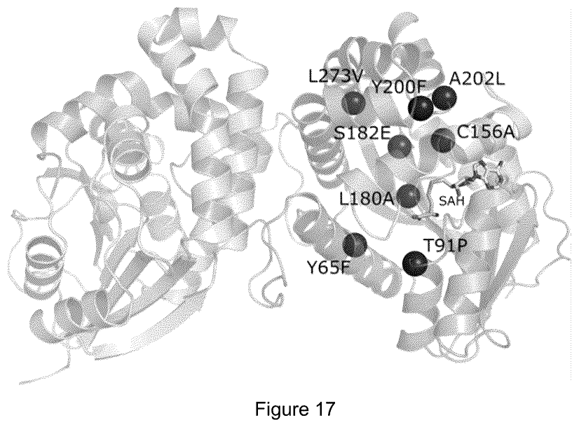

To reduce SAH inhibition, we mutated the residues (D135 and T91) that form hydrogen bonding with SAH so that the binding between SAH and the enzyme was weakened (FIG. 16a). Mutating D135 to alanine, glutamate, glycine and proline nearly abolished the enzymatic activity. Interestingly, mutating T91 to proline gave rise to marginally higher activity as compared to pMT7 (FIG. 16c). T91P with C156A was combined to create pMT10 (SEQ ID No. 12) (all the mutations are summarized in Table 8). Steady-state kinetics analysis showed that k_{cat} of pMT10 (0.051 h⁻¹) was approximately 10-fold higher than pMT7. Unexpectedly, Kₘ of SAM for pMT10 (30.5 μM) decreased as compared to pMT7. This led to increased sensitivity of pMT10 towards SAH, as shown that IC₅₀ of SAH for pMT10 decreased to 3.1 μM (Table 2).

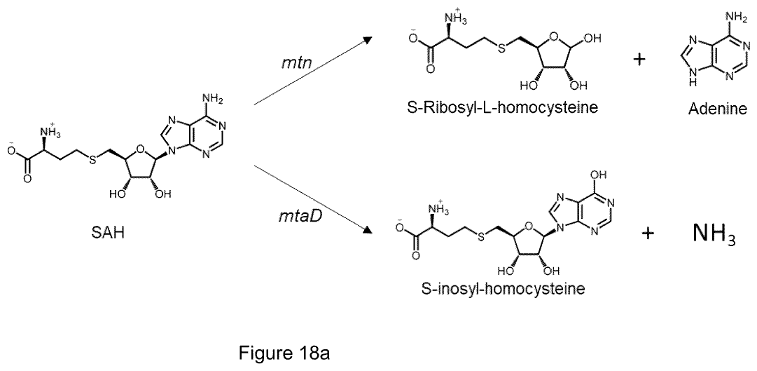

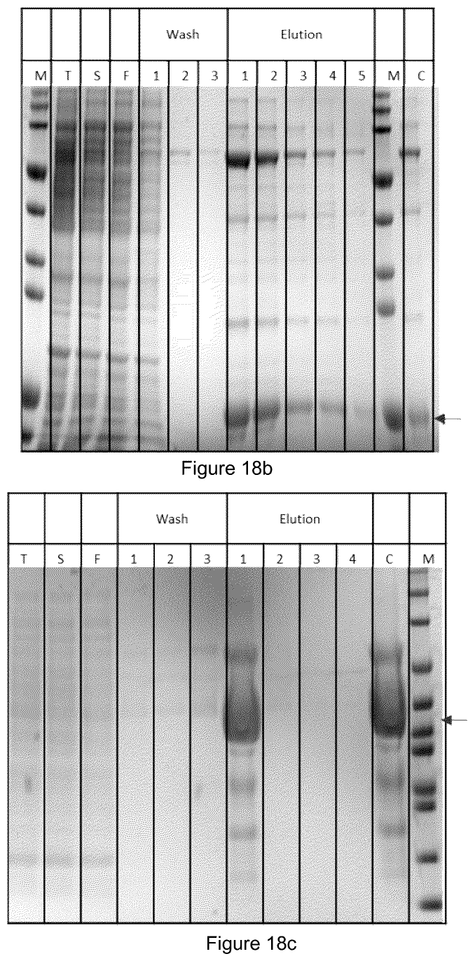

An alternative strategy to reduce SAH inhibition is to introduce auxiliary enzymes that degrade SAH12 (FIG. 18a). As shown in FIG. 3c, supplementary mtn and mtaD restored pMT7 and pMT10 activity when 20 μM SAH was introduced in the purified enzymatic reaction. However, supplementing mtn did not enhance the methyltransferase activity, probably attributed to the low purity of the purified mtn (FIG. 18b). Noteworthy, with mtaD enzyme, cis-α-irone production by pMT10 was improved by ˜2.5 fold as compared to pMT10 reaction alone (FIG. 3c). MtaD is a deaminase that modifies the adenine moiety¹³, suggesting that the amine group in adenine may play an important role in enzyme binding. This somewhat agrees with the effect of mutating D135, which abolishes the hydrogen bond interaction between pMT7 and the amine group of the adenine moiety.

With the positive effect of mtaD, in vitro biotransformation by using cell lysate containing overexpressed pMT10 and mtaD was explored. To challenge the enzyme, 10 mg/L (0.05 mM), 100 mg/L (0.5 mM) or 1000 mg/L (5 mM) of psi-ionone was supplemented into the reaction, and 6.4 mg/L (˜0.03 mM), 54 mg/L (˜0.26 mM) and 182 mg/L (˜0.88 mM) of cis-α-irone were produced, respectively after incubating the reaction at 28° C. for 3 days (FIG. 3d). It was observed that the higher the initial concentration of psi-ionone, the lower the percentage of conversion, and supplementing mtaD did not result in further improvement in cis-α-irone production. This suggests that the endogenous mtn present in E. coli extracts is sufficient to alleviate the inhibition of SAH on methyltransferase activity.

To further reduce the amount of β-irone and increase cis-α-irone production, C156 was mutated to aspartic acid instead to provide pMT11 based on pMT10. Compared to pMT10, pMT11 provided improved cis-α-irone production both by the fold change and percentage (FIG. 23). Additional mutations of N231D, and Y244A, S245A, and G267P provided pMT12 and pMT13 respectively which maintained the percentage of cis-α-irone produced while further improving the absolute amount produced (fold change) (FIG. 23). N231D was chosen to increase the stability and/or solubility of the pMT enzyme. Based on the crystal structure, a SAH molecule was observed very close to these three residues, namely Y244, S245 and G267. Y244A and S245A were chosen based on structural alignment between pMT1 and Ss.SpnF (PDB ID: 4pne). G267P was chosen based on the sequence alignment of pMT1, SaMT and ScMT. An additional mutation of E197R (pMT14) was chosen to increase the solubility/stability of the pMT enzyme, however E197R reduced the fold change of cis-α-irone to a similar level as pMT10 but with higher percentage of cis-α-irone (FIG. 23). A summary of the mutations for the numbered pMT enzymes is provided in Table 8.

FIGS. 24a and 24b show the in-vivo and in-vitro cis-α-irone production of other possible single mutations to pMT12. The in-vitro results in FIG. 24b validate the mutations and in-vivo results in FIG. 24a. The mutations in FIG. 24 include Q11H (SEQ ID No. 23), V12R (SEQ ID No. 24), A95C (SEQ ID No. 25), H123Q (SEQ ID No. 26), E127T (SEQ ID No. 27), M137T (SEQ ID No. 28), D176V (SEQ ID No. 29), D176Y (SEQ ID No. 30). P191S (SEQ ID No. 31), P191V (SEQ ID No. 32), F212L (SEQ ID No. 33), S268H (SEQ ID No. 34), AND T272A (SEQ ID No. 35). The screening results in FIGS. 24 and 25 are semi-quantitative to identify beneficial mutants that give greater than one-fold change of irone production as compared to pMT12. The mutations of Q11H (SEQ ID No. 23), D176V (SEQ ID No. 29), D176Y (SEQ ID No. 30), P191V (SEQ ID No. 32) each showed a significant increase in the in-vivo production of cis-α-irone (FIG. 24a), while the mutations of A95C (SEQ ID No. 25), P191S (SEQ ID No. 31), F212L (SEQ ID No. 33), and S268H (SEQ ID No. 34) showed a moderate increase in the in-vitro production of cis-α-irone (FIG. 24b).

FIGS. 25a to 25c (and Table 4) show the in-vivo testing results of other possible mutations that may be made to pMT12 to improve its in-vivo production of cis-α-irone. FIGS. 25a to 25c each include two dotted lines to account for a 10% error bar to show the normalized fold change of 0.9 and 1.1. The mutations of D159L (SEQ ID No. 76), D176L and A248V (SEQ ID No. 77), D176L (SEQ ID No. 78), D176V (SEQ ID No. 29), D176K (SEQ ID No. 79), D176R (SEQ ID No. 80), E190Q (SEQ ID No. 83) showed significant improvement in cis-α-irone production. Some mutations in FIGS. 25a to 25c, for example A95C, H123Q, D176V, P191V, F212L, S268H, were screened twice and showed similar qualitative results in FIG. 24a and confirmed that most of these mutations were beneficial.

TABLE 4
Fold change in in-vivo cis-α-irone production of mutations in FIGS. 25a to 25c

| SEQ ID | | Fold | SEQ ID | | Fold | SEQ ID | | Fold |
|---|---|---|---|---|---|---|---|---|
| No. | Mutation | change | No. | Mutation | change | No. | Mutation | change |
| 36 | Q11L | 0.92 | 59 | A95L | 0.92 | 81 | T185V | 1.14 |
| 37 | Q11P | 0.92 | 60 | A107S | 1.17 | 82 | T185L | 1.07 |
| 38 | Q11M | 0.90 | 61 | A107G | 0.95 | 83 | E190Q | 2.90 |
| 39 | Q11V | 0.88 | 62 | H123N | 0.91 | 84 | E190A | 1.12 |
| 40 | Q11W | 0.90 | 26 | H123Q | 0.87 | 85 | E190P, | 1.02 |
| | | | | | | | G122S | |
| 41 | V12K | 1.00 | 63 | H123S | 0.84 | 86 | P191L | 1.12 |
| 42 | V12A | 0.97 | 64 | T126S | 0.96 | 87 | P191I | 1.31 |
| 43 | V12G | 0.97 | 65 | T126E | 0.88 | 88 | P191Y | 1.29 |
| 44 | T13L | 0.97 | 66 | E127G | 0.98 | 89 | P191K | 1.26 |
| 45 | T13M | 0.97 | 67 | L129C | 1.08 | 32 | P191V | 1.26 |
| 46 | T13Q | 0.93 | 68 | L129K | 1.00 | 90 | E192S | 0.97 |
| 47 | T13A | 0.91 | 69 | L129V | 1.03 | 91 | L195I | 1.08 |
| 48 | T13G | 0.91 | 70 | M137G | 1.15 | 33 | F212L | 1.11 |
| 49 | A14M | 0.94 | 71 | M137A | 1.15 | 92 | L195V | 1.02 |
| 50 | A14R | 0.93 | 72 | M137D | 1.13 | 93 | S268K | 1.35 |
| 51 | A14G | 0.92 | 73 | M137H | 1.11 | 94 | S268Q | 1.32 |
| 52 | A14P | 0.92 | 74 | M137N | 1.11 | 34 | S268H | 1.26 |
| 53 | A14L | 0.92 | 75 | M137S | 1.11 | 95 | S268R | 1.23 |
| 54 | A14T | 0.90 | 76 | D159L | 1.55 | 96 | S268L | 1.18 |
| 55 | K94R | 0.97 | 77 | D176L, | 3.14 | 97 | A269L | 1.29 |
| | | | | A248V | | | | |
| 56 | K94V | 0.84 | 78 | D176L | 2.52 | 98 | A269G | 1.23 |
| 57 | A95I | 0.98 | 29 | D176V | 2.24 | 99 | A269R | 1.22 |
| 25 | A95C | 0.95 | 79 | D176K | 1.90 | 100 | A269Y | 1.21 |
| 58 | A95V | 0.94 | 80 | D176R | 1.80 | [symbol] | pMT12 | 1 |

### In Vivo Biotransformation of Glucose to Cis-α-Irone

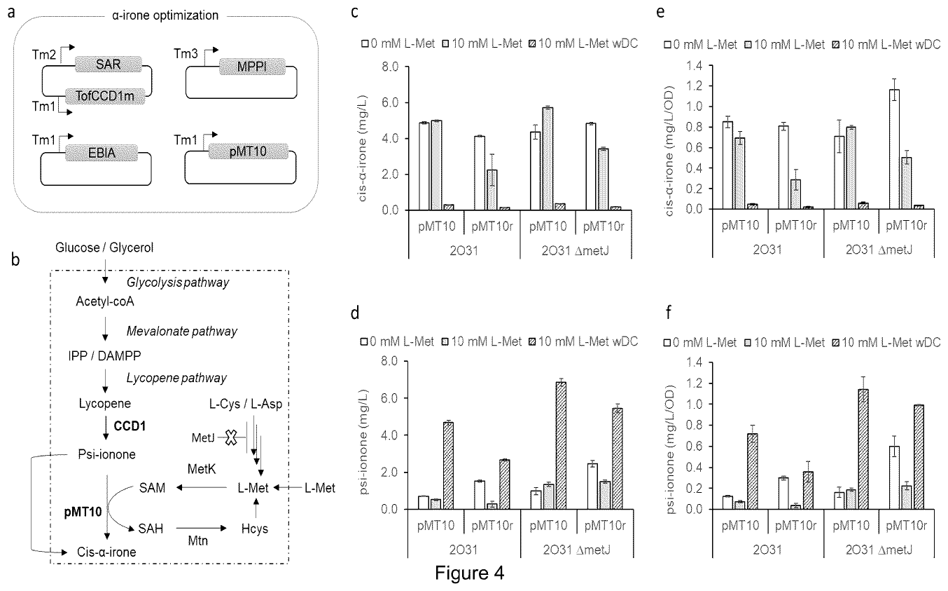

The advantage of in vivo biotransformation is that microbial cells can regenerate expensive co-factors rendering the scale-up bioprocess more cost-effective. Moreover, microbial cells possess sophisticated mechanisms to regulate SAH concentration¹⁴, so it may alleviate the inhibition of SAH to pMT enzyme. Thus, pMT was incorporated into our psi-ionone producing microbe^{7,8}, to synthesize cis-α-irone from cheap renewable carbon sources (glucose/glycerol) (FIG. 4 and FIG. 19). To achieve high productivity, the psi-ionone production was first optimized by multidimensional heuristic process (MHP)¹⁵, and strain 2O31 was identified to be the highest producer for psi-ionone (FIG. 19b). 2O31 was used as the base strain to probe cis-α-irone production in vivo (Table 5). Moreover, to improve the plasmid stability, auxotrophic strain was used with chemically defined media¹. The plasmid carrying pMT1, pMT7 or pMT10 was transformed into the strain 2O31, and produced less than 0.01, about 2 and about 3 mg/L cis-α-irone respectively in 2 days (FIG. 19d). To further optimize irone production with pMT10, three different culture conditions were tested: no methionine or dodecane added (control); 10 mM methionine added; 10 mM methionine and 20% dodecane added. In the control reaction, approximately 4.8 mg/L or 0.85 mg/L/OD cis-α-irone was produced after 3-day incubation (2O31-pMT10, FIGS. 4c and 4e). When 10 mM methionine was added to increase SAM pool, it did not increase irone titer and reduced the specific irone titer (2O31-pMT10, FIGS. 4c and e). Lastly, when both methionine and dodecane were added, psi-ionone was accumulated in the organic layer and only ˜0.3 mg/L or ˜0.05 mg/L/OD cis-α-irone was produced (2O31-pMT10, FIGS. 4c, d and e). This observation suggested that psi-ionone was secreted to the media first and subsequently taken up by the cells for methylation (FIG. 4b). The limited methylation efficiency might be due to insufficient cofactor or SAH inhibition. Hence, Metk and mtn was co-expressed to convert methionine to SAM and hydrolyze SAH (2O31-pMT10r) and the three conditions were tested again. Unexpectedly, cis-α-irone concentration was reduced under all three conditions (2O31-pMT10r, FIGS. 4c and e). In fact, the total amount of psi-ionone and cis-α-irone decreased (FIG. 4c, and d). As SAM biosynthesis is regulated by metJ in E. coli^{16,17}, the aforementioned six reactions were subsequently examined under metJ deleted genetic background (2O31, ΔmetJ). An increase in cis-α-irone titer to ˜5.7 mg/L was produced by 2O31 ΔmetJ-pMT10 strain with 10 mM methionine supplementation (FIG. 4c). An increase in specific cis-α-irone titer to ˜1.2 mg/L/OD was produced by 2O31 ΔmetJ-pMT10r strain without methionine supplementation (FIG. 4d). It is noted that psi-ionone concentration was generally higher in 2O31 ΔmetJ strain as compared to 2O31 strain, suggesting that irone concentration could be potentially increased (FIGS. 4d and 4f).

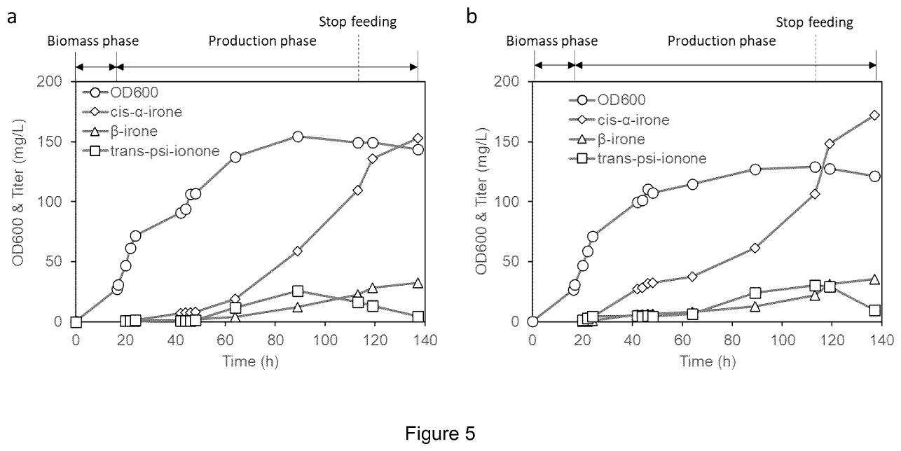

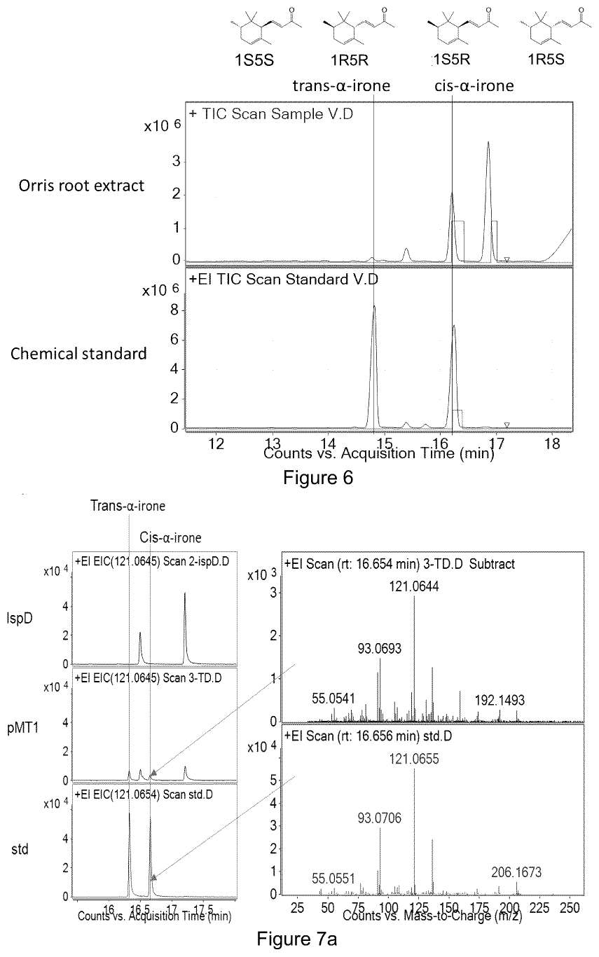

Lastly, a single-phase fed-batch process was tested in a 5 L bioreactor. Even though 2O31 ΔmetJ strain produced a higher titer of cis-α-irone, its growth seemed to be impeded during fermentation. Thus, 2O31-pMT10 strain was tested. The optimized bioprocess for α-ionone was applied⁸, in which the glucose feeding rate was set at 10 g/h and the glucose concentration was kept between 0-5 g/L. Two different dissolved oxygen (DO) levels were tested: 2% and 10%. Under these DO settings, evaporation was kept at minimum which is <0.05%. As shown in FIG. 5, cis-α-irone was produced faster at 10% DO than 2% DO on the first day post-induction. Subsequently, cis-α-irone production became faster at 2% DO than 10% DO. At 113 h, when glucose feeding was stopped, approximately 109 mg/L and 106 mg/L cis-α-irone were produced at 2% and 10% DO respectively. By continuing incubating the cultures for another 24 h, a final titer of approximately 153 mg/L and 172 mg/L cis-α-irone were produced at 2% and 10% DO respectively. Simultaneously, a decrease in psi-ionone concentration was observed during the last 24 h, indicating psi-ionone was converted to cis-α-irone. Other DO levels may also be used, for example 15%. A higher DO level may lead to more ATP/NADH generation and likely higher irone production; however, a higher aeration rate may increase the loss of irone by evaporation. Hence, the DO level may need to be adjusted accordingly for optimum production levels.

### Methylation on Structurally Similar Substrates

Using E. coli cell lysate overexpressing pMT6, a range of structurally similar terpenoids for methylation and cyclization reaction was assayed (FIG. 21a). E. coli lysate overexpressing green fluorescence protein was used as a control. SPME-GCMS analysis detected methylated products in the pMT6 reactions with linalool (FIG. 21b), geraniol (FIG. 21c) and geranylacetone (FIG. 21d). Citral was not stable and was reduced to geraniol in the presence of cell lysate. And no methylated product was detected when geranylacetate was used. Compound identity was searched against the National Institute of Standards and Technology (NIST) library. It is found that pMT6 prefers to transfer the methyl group to the hydroxy group in the alcohol terpenes (linalool and geraniol) to form methoxy ether rather than methylate the terminal alkene group. When the alcohol group was esterified as the acetate ester, pMT6 failed to methylate the substrate, geranylactate. Lastly, for the monoterpene ketone, geranylacetone, with saturated C7-C8 bond, multiple methylated products were detected based on the molecular ion. Based on the NIST prediction, it was suggested that geranylacetone was methylated and cyclized.

### Methods

### Strains, Plasmids, and Chemicals

E. coli BI21-Gold DE3 strain (Stratagene) was used in this study. CRISRP-cas9 mediated gene deletion was carried out to modify the genome of E. coli BL21 strain¹⁸. pET11a (Novagen) was used to construct the methyltransferase mutants. For in vivo psi-ionone production, the plasmids were modified p15A plasmids as previously described (Table 5) 7 and is exemplified by SEQ ID Nos. 104 to 117. The genes: pMT1 or TleD from Streptomyces blastmyceticus, SaMT from Streptomyces albireticuli, and ScMT11 from Streptomyces clavuligerus were codon optimized and synthesized by Integrated DNA Technologies. Mtn was amplified from Escherichia coli genome. Unless otherwise noted, all chemicals and reagents were obtained from Sigma-Aldrich.

TABLE 5
Strains and plasmids used herein.

| Name | Description | Reference | Remarks |
|---|---|---|---|
| E. coli BL21- | F⁻ ompT hsdS (r_{B}⁻ m_{B}⁻) dcm⁺ Tet^{r} | Stratagene | For in vitro enzyme |
| Gold (DE3) | gal λ(DE3) endA Hte | | expression |
| E. Coli | F−, endA1, supE44, thi-1, recA1, | Clontech | For plasmid |
| Stellar | relA1, gyrA96, phoA, ϕ80d lacZΔ | | construction |
| Competent | M15, Δ(lacZYA-argF) U169, Δ(mrr- | | |
| Cells | hsdRMS-mcrBC), ΔmcrA, λ- | | |
| 2O31 strain | BL21, ΔaroA, ΔaroB, ΔaroC, | This study | Auxotrophic strain. |
| | ΔserC, carrying plasmids p15A-spec- | | Base strain to |
| | Tm2-hmgS-atoB-hmgR-Tm1-OfCCD1m, p15A- | | produce psi-ionone |
| | cam-Tm3-mevK-pmk-pmd-idi, p15A-kan- | | and transform pMT |
| | Tm1-crtEBI-ispA. | | enzymes. |
| 2O31 pMT10 | 2O31 strain transformed with p15A- | This study | Strain for α-irone |
| | amp-Tm1-pMT10 | | production. |
| 2O31 pMT10r | 2O31 strain transformed with p15A- | This study | Strain for α-irone |
| | amp-Tm1-pMT10-metK-mtn | | production. |
| 2O31 ΔmetJ | 2O31 strain with metJ deletion and | This study | Strain for α-irone |
| pMT10 | transformed with p15A-amp-Tm1-pMT10 | | production. |
| 2O31 ΔmetJ | 2O31 strain with metJ deletion and | This study | Strain for α-irone |
| pMT10r | transformed with p15A-amp-Tm1-pMT10- | | production. |
| | metK-mtn | | |
| p15A-spec-Tm2- | Plasmid for overexpression of | Reference ⁷ | Module 1 and |
| hmgS-atoB- | hmgs, atoB, thmgR genes, | | module 4 |
| hmgR-Tm1- | controlled by mutated Tm2 | | |
| OfCCD1m | promoter, and modified OfCCD1, | | |
| | controlled by mutated Tm1 | | |
| | promoter. It carries spectinomycin | | |
| | resistance gene. | | |
| p15A-cam-Tm3- | Plasmid for overexpression of | Reference ⁷ | Module 2 |
| mevK-pmk-pmd- | mevk, pmk, pmd, idi genes, | | |
| idi | controlled by Tm3 promoter. | | |
| | It carries chloramphenicol | | |
| | resistance gene. | | |
| p15A-kan-Tm1- | Plasmid for overexpression of crtE, | Reference ⁷ | Module 3 |
| crtEBI-ispA | crtB, crtI and ispA genes, controlled | | |
| | by Tm1 promoter. It carries | | |
| | kanamycin resistance gene. | | |
| p15A-amp-Tm1- | Plasmid for overexpression of | This study | Module 5 |
| eGFP | eGFP gene, controlled by Tm1 | | |
| | promoter. It carries ampicillin | | |
| | resistance gene. | | |
| p15A-amp-Tm1- | Plasmid for overexpression of | This study | Module 5 |
| pMT1 | pMT1 gene, controlled by Tm1 | | |
| | promoter. It carries ampicillin | | |
| | resistance gene. | | |
| p15A-amp-Tm1- | Plasmid for overexpression of | This study | Module 5 |
| pMT7 | pMT7 gene, controlled by Tm1 | | |
| | promoter. It carries ampicillin | | |
| | resistance gene. | | |
| p15A-amp-Tm1- | Plasmid for overexpression of | This study | Module 5 |
| pMT10 | pMT10 gene, controlled by Tm1 | | |
| | promoter. It carries ampicillin | | |
| | resistance gene. | | |
| p15A-amp-Tm1- | Plasmid for overexpression of | This study | Module 5 |
| pMT10-metK-mtn | pMT10, metK, mtn genes, controlled | | |
| | by Tm1 promoter. It carries | | |
| | ampicillin resistance gene. | | |

### Cloning and Site-Directed Mutagenesis

Mutations were carried out with modified QuikChange™ protocol⁷. Briefly, overlapping primers were designed, which carried the desired mutation, to amplify the plasmid carrying the pMT gene. 10 μL of polymerase chain reaction (PCR) was carried out to amplify the plasmid by high-fidelity iproof polymerase (BioRad). Subsequently, the template plasmids were removed by treating the PCR reaction mixture directly with 0.5 unit of DpnI enzyme (New England Biolabs) at 37° C. for 3 h. Lastly, 1 μL of PCR reaction mixture was transformed into 20 μL of Stellar competent cells (Clonetech) by heat-shock transformation. The mutation was verified by sequencing.

For the pooled colony screening, the cloning steps were the same as the directed mutagenesis except degenerative primers with NNK or MNN were used to amplify the plasmid. After transformation, all the colonies were combined with 1 ml phosphate buffer saline solution (PBS) and subjected to plasmid extraction. The purified plasmid was then transformed into BL21 cells and plated on agar with ZYM auto-induction media and incubated at 20° C. for 3 days¹⁹. The colonies formed were collected in 1 ml PBS, and OD was measured. 12.5*250 μl*OD cells were taken for subsequence reaction with psi-ionone to test methyltransferase activity.

### Protein Purification

His-tag protein purification was carried out according to the manufacturer's instruction (Cube Biotech). For each enzyme, it was overexpressed in 200 ml of ZYM autoinduction media with 5 mM lactose at 20° C. for 36 hrs. Following that, cells were pelleted and resuspended with 10 ml of His-binding buffer (50 mM Tris, pH 8, 0.5M NaCl and 20 mM imidazole) supplemented with 1 mg/ml lysozyme and 3 U/ml of benzonase nuclease (Merck) and treated for 1 hour (hr) at 20° C., 300 rpm. The cells were further lysed by 2 cycles of freeze-thawing with freezing at −80° C. for 4 hrs and thawing at room temperature for 45 mins. The supernatant (soluble fraction) was obtained by centrifuging at 15,000 g for 30 mins at 4° C. The collected supernatant was then mixed with 1 ml of equilibrated Ni-NTA beads (Cube Biotech) overnight at 4° C. with continuous mixing. The next day, the beads were washed 3 times with 5 ml of His-binding buffer to remove any unbound proteins before eluting bound enzyme 5 times with 0.5 ml of His-elution buffer (50 mM Tris, pH8, 0.5M NaCl, 0.5M imidazole). The eluted protein was concentrated using spin-column with a MW cut-off of 10 kDa (Sartorius). Protein quantification and purity were determined through micro BCA assay (Pierce) and running SDS-PAGE, respectively.

### Western Blot Analysis

To quantify the protein concentration in soluble fraction of cell extracts, western blot analysis was carried out. Standard SDS-PAGE protocol was performed before transferring the proteins onto nitrocellulose membrane using iBolt2 dry transfer system (Thermofisher). The membrane was then blocked with 20 ml of 5% milk in TBST buffer for 1 hr at room temperature. Following that, it was probed with anti-6×His-tag antibody-HRP (Abcam) in 1% milk at a ratio of 1:2000 overnight at 4° C. The membrane was washed 3 times with 20 ml of TBST buffer before adding in substrate for chemiluminescence detection (Millipore) and imaging using ChemiDoc system (Bio-rad). The chemiluminescence signal was quantified using Image Lab (BioRad). Purified pMT enzyme with known concentration (2.8-90 ng/μl) was used to prepare the standard curve. The concentration of pMT enzyme in cell extract was then quantified against the standard curve.

### Purified Enzyme Reaction

Following enzyme purification, a 100 μl enzymatic reaction was setup with a final concentration of 0.5 mg/ml of enzyme, 40 mg/L psi-ionone, 0.2 mM SAM, 100 mM Tris (pH 7), 10 mM MgCl₂ and 15 mM NaCl. The reaction was done in 0.2 ml PCR strips and incubated at 28° C., 1200 rpm overnight. Alongside, a control without enzyme (with 40% glycerol only) was included as negative control. The next day, cis-α-irone was extracted with 100 μl of ethyl acetate and 50 μl organic layer taken for GCMS analysis. For enzyme characterization, similar enzymatic reaction was performed, with varying concentration of psi-ionone (2-40 mg/L) and SAM (10-100 μM), and the reaction was stopped after 2 h incubation at 28° C. to ensure the enzymatic conversion is still within linear range.

### Cell Extracts Preparation and Reaction

For each pMT mutant, it was transformed into BL21 and grown in autoinduction media with 5 mM lactose at 20° C. for 36 hrs. Then, cells were pelleted and concentrated 10 times by adding 1 ml of PBS. The OD₆₀₀ was measured and 40*600 OD*ul cells were transferred into a new 1.5 ml tube. The cells were spun down and resuspended in 600 μl of lysis buffer (1×DNase buffer, 150 mM NaCl, 0.5% glycerol, 1 mg/ml lysozyme and 20U of DNasel). The cells were incubated for 2 hrs at 20° C. with shaking at 300 rpm. Following that, 2 cycles of freeze-thaw was done to completely lyse the cells. At the end of the 2^{nd} thawing, the cell lysate was used to setup a 1 ml reaction in 2 ml GC vials. The reaction consisted of 50 mM Tris (pH7), 10 mM MgCl₂, 60 mM NaCl, 0.2 mM SAM and 10 mg/L of psi-ionone and caryophyllene each. To this, 500 μl of cell lysate was added while the remaining 100 μl was used for SDS-PAGE to analyze the total and soluble protein expression. The reaction was incubated for 2 days at 28° C., 300 rpm prior to extraction of cis-α-irone with 500 μl hexane. From this, 20 μl of organic layer was taken into 180 μl hexane for GCMS analysis.

Biotransformation of Glucose/Glycerol into Cis-α-Irone

The four plasmids were transformed into E. coli BL21 (DE3), ΔaroA, ΔaroB, ΔaroC, ΔserC or E. coli BL21 (DE3), ΔaroA, ΔaroB, ΔaroC, ΔserC, ΔmetJ strains and plated on Agar plate containing LB media (10 g, tryptone. 5 g, yeast extract. 10 g, NaCl) supplemented with appropriate antibiotics (100 mg/L ampicillin, 34 mg/L chloramphenicol, 50 mg/L kanamycin, and 100 mg/L spectinomycin). One colony was picked and inoculate into LB media with antibiotics. 1% of overnight culture was inoculated into 1 ml fresh auto-inducing chemically defined media. The chemical media contains carbon source solution: 0.5 g/L glucose, 10 g/L glycerol; inducer: 30 mM lactose and base media: 2 g/L ammonium sulfate, 4.2 g/L KH₂PO₄, 11.24 g/L K₂HPO₄, 1.7 g/L citric acid, 0.5 g/L MgSO₄, and 10 mL/L trace element solution. The trace element solution (100×) contained 0.25 g/L CoCl₂·6H₂O, 1.5 g/L MnSO₄·4H₂O, 0.15 g/L CuSO₄·2H₂O, 0.3 g/L H₃BO₃, 0.25 g/L Na₂MoO₄·2H₂O, 0.8 g/L Zn(CH₃COO)₂, 5 g/L Fe(III) citrate, and 0.84 g/L ethylenediaminetetra-acetic acid (EDTA) at pH 8.0. The culture was grown at 28° C. for 3 days and the products were extracted with 1 ml hexane for GCMS analysis. An example of a fed-batch process for α-ionone Is briefly described, overnight culture was inoculated into 2 L chemical media comprising the base media and 5 g/L glucose, at 37° C. and 30% DO. pH was controlled at 7 with base solution (14% ammonia and 0.5 M NaOH). Feed media (500 g/L glucose and 5 g/L MgSO₄) was added into bioreactor once OD₆₀₀ reached 5, at a rate of 7.15-25.75 g/h glucose for about 4-5 h until OD₆₀₀ reached ˜30-40. Subsequently, 0.1 mM IPTG was added to induce the production, and feeding rate was kept at a constant rate of 10 g/h glucose, and the temperature was reduced to 30° C. DO was adjusted to 2% or 10%. Feeding was stopped at 113 h when 2 L feeding media was finished, but the culture was further incubated for 24 h. To capture the evaporated products, the exhaust was connected to 25 ml sunflower oil.

### Gas Chromatography Mass Spectrum Analysis

For the mutant pMT activity screening, the reactants and products were analyzed by head space solid phase microextraction (HS-SPME) coupled with an Agilent 5977B gas chromatography (GC) system equipped with Agilent DB-5 ms column (30 m×250 μm×0.25 μm) and mass spectrometry (MS) with high efficiency source. The reaction mixtures were carried out in SPME vials which were incubated at 60° C. for 20 mins to allow the release of volatile compounds. The absorbent fiber (50/30 μm divinylbenzol/carboxen/polydimethyl-SPME fiber, SUPELCO) was then exposed to the headspace of the vial for 20 mins. The extracted analytes were desorbed at the GC inlet at 250° C. for 1 min and injected into GC with a split ratio of 200:1. The GC oven temperature increased from 50° C. to 140° C. at a rate of 10° C./min and held at 140° C. for 10 min. Subsequently, the temperature was increased to 320° C. at a rate of 60° C./min and held at 320° C. for 2 mins. The concentration of psi-ionone and irone were calculated by interpolating with a standard curve prepared by synthetic standards.

To determine the purified enzyme kinetics and in vivo production, the reaction mixture was extracted with 0.5× or 1× volume of ethyl acetate and the organic phase was diluted appropriately before subjected to GCMS analysis. The samples were analyzed on an Agilent Intuvo 9000 GC system equipped with Agilent DB-WAX Ultra Inert Intuvo GC column (30 m×250 μm×0.25 μm) and Agilent 5977B mass spectrometry with high efficiency source. 1 μl organic phase was injected at the split ratio 10:1 at 250° C. The oven temperature was held at 50° ° C. for 1 min and increased to 200° C. at a rate of 40° C./min and held at 200° C. for 3 min. Subsequently, the temperature increased to 230° C. at a rate of 40° C./min and held at 230° ° C. for 5 mins. The concentration of psi-ionone and irone were calculated by interpolating with a standard curve prepared by synthetic standards.

### Computational Studies

The three-dimensional structure of pMT1 in complex with the cofactor S-adenosyl-I-homocysteine with the substrate teleocidin A1 was retrieved from the PDB database (PDB id: 5GM2). One dimeric unit was used as template for modelling pMT1 (wild-type) in complex with the four α-irone isomers (1R5R and 1S5S for the trans-α-irone and 1S5R and 1R5S for the cis-α-irone). The α-irones were first built and gradually relaxed using the Avogadro software version 1.1.1²⁰. They were then manually docked into pMT1 active site using the bound substrate teleocidin A1 as template from crystallographic structure. pMT3 (Y200F, S282E) and pMT4 (Y200F, S282E, L273V) were modelled using Modeller 9.19²¹ and 3D models with lowest DOPE score were kept for further analyses.

The corresponding complexes of pMT3 or pMT4 with the four α-irones were built by superposing pMT1 complexes.

MD simulations were performed using the AMBER ff14S force-field²² for enzymes and GAFF²³ for the ligands using pmemd.CUDA of AMBER16 software²⁴. The partial charges for the ligand were computed using AM1-BCC method²⁵ from antechamber. The system was protonated using propka webserver to set the experimental pH of 7. MD simulation parameters were the same as previously described²⁶. In all stages, the sulphur of SAH was kept close to the irone isomer by setting a distance constraint lower than 5 Å and using a force constant of 25 kcal mol-1 Å-2.

The MD simulations were carried out for a total of 5 ns for all complexes. MM/PBSA calculations were performed using MMPBSA.py software and default parameters for Poisson-Boltzmann as described by Miller et al.²⁷

TABLE 6
Summary of directed evolution of pMT enzyme to produce cis-a-irone from psi-
ionone.

| | | | | Changes |
|---|---|---|---|---|
| Round | Name | Parent | Diversification strategy | made |
| 1 | pMT2 | pMT1 or TIeD from | Pooled colony screening to increase cis-α- | Y200F |
| | | Streptomyces | irone production. Site-saturation | |
| | | blastmyceticus | mutagenesis, Y200X | |
| 2 | pMT3 | pMT2 | Pooled colony screening to increase | S182E |
| | | | cis/trans-α-irone ratio. Site-saturation | |
| | | | mutagenesis, S182X | |
| 3 | pMT4 | pMT3 | Computer-aided structural analysis and | L273V |
| | | | site-saturation mutagenesis, L273X | |
| 4 | pMT5 | pMT4 | Pooled colony screening to increase cis-α- | L180A |
| | | | irone production. Site-saturation | |
| | | | mutagenesis, L180X | |
| 5 | pMT6 | pMT5 | Structural analysis to reduce the binding | A202L |
| | | | pocket, A202S, A202V, A202L, A202F | |
| 6 | pMT7 | pMT6 | Pooled colony screening to reduce B-irone | Y65F |
| | | | production. Site-saturation mutagenesis, | |
| | | | Y65X | |
| 7 | pMT8 | pMT6 | Pooled colony screening to reduce B-irone | C156P |
| | | | production. Site-saturation mutagenesis, | |
| | | | C156X | |
| 8 | pMT9 | pMT7 | Combining positive C156X mutation on top | C156A |
| | | | of pMT7, C156A, C156P | |
| 9 | pMT10 | pMT9 | Remove the hydrogen bond between SAH | T91P |
| | | | and pMT enzyme. Stability analysis by | |
| | | | Hotspot Wizard 3.0²⁹. T91P | |
| 10 | pMT11 | pMT10 | Re-screen C156 residue to reduce ß-irone | C156D |
| | | | production. Site-directed mutagenesis, | |
| | | | C156D | |
| 11 | pMT12 | pMT11 | Increase the solubility and stability of pMT | N231D |
| | | | enzyme. Site-directed mutagenesis. | |
| 12 | pMT13 | pMT12 | Reduce the allosteric interaction between | Y244A, |
| | | | pMT and SAH. Site-directed mutagenesis. | S245A, |
| | | | | G267P |
| 13 | pMT14 | pMT13 | Increase the solubility and stability of pMT | E197R |
| | | | enzyme. Site-directed mutagenesis. | |

TABLE 7
Summary of fold change in cis-α-irone produced by each mutant pMT enzyme as shown in FIG. 20

| | pMT1_R160 | pMT1_Y200 | pMT1_M236 | pMT1_E153 | pMT1_S182 | pMT2_C156 | pMT2_L180 | pMT2_S182 | pMT2_F196 |
|---|---|---|---|---|---|---|---|---|---|
| A | 1.7 | 0.2 | 0.7 | 0.1 | | 0.6 | 1.4 | 0.1 | 0.2 |
| C | 1.5 | 0.1 | 4.7 | 0.0 | | 1.0 | 2.7 | 0.1 | 0.1 |
| D | 0.0 | 0.0 | 0.0 | 0.2 | | 0.1 | 0.1 | 0.1 | 0.1 |
| E | 0.0 | 0.0 | 0.9 | 1.0 | 0.84 | 0.1 | 0.9 | 5.4 | 0.2 |
| F | 0.0 | 12.2 | 0.0 | 0.0 | | 0.5 | 0.4 | 0.1 | 1.0 |
| G | 0.0 | 0.1 | 0.0 | 0.0 | | 0.0 | 0.3 | 0.2 | 0.4 |
| H | 1.0 | 0.2 | 2.6 | 0.0 | | 0.3 | 0.2 | 0.2 | 0.3 |
| I | 1.5 | 2.0 | 0.5 | 0.0 | | 0.6 | 0.8 | 0.1 | 0.1 |
| K | 0.0 | 0.0 | 0.0 | 0.0 | | 0.0 | 0.1 | 0.0 | 0.2 |
| L | 0.9 | 0.4 | 1.1 | 0.0 | | 0.6 | 1.0 | 0.1 | 0.3 |
| M | 0.9 | 0.4 | 1.0 | 0.0 | | 0.2 | 0.8 | 0.3 | 0.5 |
| N | 0.8 | 0.0 | 3.9 | 0.0 | | 0.0 | 0.4 | 1.6 | 0.4 |
| P | 0.6 | 0.0 | 0.0 | 0.0 | | 0.5 | 0.0 | 0.1 | 0.2 |
| Q | 1.3 | 0.1 | 0.0 | 0.0 | | 0.2 | 0.4 | 1.0 | 1.0 |
| R | 1.0 | 0.0 | 0.0 | 0.0 | | 0.0 | 0.0 | 0.0 | 3.0 |
| S | 0.0 | 0.2 | 0.7 | 0.0 | 1.0 | 0.1 | 0.2 | 1.0 | 0.4 |
| T | 1.2 | 0.0 | 1.7 | 0.0 | 0.23 | 0.6 | 0.3 | 1.3 | 0.3 |
| V | 1.5 | 1.1 | 0.7 | 0.0 | | 0.3 | 0.7 | 0.2 | 0.1 |
| W | 0.7 | 2.1 | 0.2 | 0.0 | | 0.1 | 0.1 | 0.1 | 0.2 |
| Y | 1.3 | 1.0 | 0.0 | 0.0 | | 0.0 | 0.0 | 0.1 | 0.6 |

| | pMT2_V36 | pMT2_L232 | pMT2_M236 | pMT2_T277 | pMT3_E153 | pMT3_L273 | pMT4_L180 | pMT4_T277 |
|---|---|---|---|---|---|---|---|---|
| A | 0.5 | 0.0 | | 0.90 | 0.0 | 0.1 | 1.8 | 0.54 |
| C | 0.4 | 0.2 | | | 0.0 | 0.5 | 1.3 | |
| D | 0.1 | 0.0 | | | 0.0 | 0.0 | | |
| E | 0.6 | 0.2 | | | 1.0 | 0.1 | | |
| F | 0.1 | 0.1 | | | 0.0 | 0.1 | | |
| G | 0.1 | 0.5 | | | 0.0 | 0.2 | | |
| H | 0.1 | 0.9 | 0.0 | | 0.0 | 0.1 | | |
| I | 0.9 | 0.2 | | 0.31 | 0.0 | 1.5 | | |
| K | 0.0 | 0.0 | | | 0.2 | 0.6 | | |
| L | 0.8 | 1.0 | | 0.08 | 0.0 | 1.0 | 1 | |
| M | 0.3 | 0.2 | 1.0 | | 0.0 | 0.3 | | |
| N | 0.0 | 0.1 | 0.1 | | 0.0 | 0.6 | | |
| P | 0.0 | 0.0 | | | 0.0 | 0.0 | | |
| Q | 0.5 | 0.2 | | | 0.0 | 0.1 | | |
| R | 0.0 | 0.0 | | | 0.0 | 0.0 | | |
| S | 0.4 | 0.0 | | | 0.0 | 0.1 | | |
| T | 0.4 | 0.0 | | 1.0 | 0.0 | 0.1 | | 1.0 |
| V | 1.0 | 0.1 | | | 0.0 | 1.5 | | |
| W | 0.1 | 0.0 | | | 0.0 | 0.1 | | |
| Y | 0.0 | 0.0 | | | 0.0 | 0.0 | | |

| | pMT5_A102 | pMT5_A202 | pMT5_T104 | pMT6_Y65 | pMT6_C156 | pMT7_T91 |
|---|---|---|---|---|---|---|
| A | 1.0 | 1.0 | | 0.5 | 0.8 | 0.3 |
| C | | | | 0.3 | 1.0 | 0.5 |
| D | 1.1 | | | 0.0 | 0.4 | 0.0 |
| E | 1.1 | | | 0.0 | 0.1 | 0.0 |
| F | | 0.18 | | 1.4 | 0.0 | 0.1 |
| G | | | | 0.1 | 0.8 | 0.5 |
| H | | | | 0.0 | 0.0 | 0.0 |
| I | 0.7 | | 0.10 | 0.3 | 0.2 | 0.2 |
| K | 1.0 | | | 0.0 | 0.0 | 0.0 |
| L | | 4.3 | | 1.0 | 0.1 | 0.1 |
| M | | | | 0.8 | 0.5 | 0.7 |
| N | | | | 0.0 | 0.1 | 0.0 |
| P | | | | 0.0 | 1.1 | 1.7 |
| Q | | | | 0.4 | 0.4 | 0.0 |
| R | 0.9 | | | 0.0 | 0.0 | 0.0 |
| S | | 1.05 | | 0.0 | 0.7 | 0.3 |
| T | | | 1 | 0.3 | 0.3 | 1.0 |
| V | 1.0 | 1.1 | 0.29 | 0.1 | 0.4 | 0.2 |
| W | | | | 0.2 | 0.0 | 0.0 |
| Y | | | | 1.0 | 0.0 | 0.0 |

TABLE 8
Summary of modified enzymes

| Name | SEQ ID No. | Mutations |
|---|---|---|
| pMT2 | 4 | Y200F |
| pMT3 | 5 | Y200F S182E |
| pMT4 | 6 | Y200F S182E L273V |
| pMT5 | 7 | Y200F S182E L273V L180A |
| pMT6 | 8 | Y200F S182E L273V L180A A202L |
| pMT7 | 9 | Y200F S182E L273V L180A A202L Y65F |
| pMT8 | 10 | Y200F S182E L273V L180A A202L C156P |
| pMT9 | 11 | Y200F S182E L273V L180A A202L Y65F C156A |
| pMT10 | 12 | Y200F S182E L273V L180A A202L Y65F C156A T91P |
| pMT11 | 13 | Y200F S182E L273V L180A A202L Y65F C156D T91P |
| pMT12 | 14 | Y200F S182E L273V L180A A202L Y65F C156D T91P N231D |
| pMT13 | 15 | Y200F S182E L273V L180A A202L Y65F C156D T91P N231D |
| | | Y244A S245A G267P |
| pMT14 | 16 | Y200F S182E L273V L180A A202L Y65F C156D T91P N231D |
| | | Y244A S245A G267P E197R |

TABLE 9
Comparison of yield by natural extraction and biomanufacturing
methods to produce cis-a-irone. Biomanufacturing method
is 7,200-33,000-fold more efficient as compared to natural
extraction given the same land area and duration.

| Manufacturing | Extraction | Biomanufacturing |
|---|---|---|
| methods | (Native pathway) | (This work) |
| Natural? | Yes | Yes |
| Stereoisomers | 3 | 2 |
| Production | 3-6 years | 7-day fermentation |
| period | | 1 year for glucose |
| | | production |
| Yield | 30-70 mg/kg | 675 mg/kg glucose |
| | Orris root | |
| Land area/kg | 10 m² | 0.04 m² |
| raw material | | |

### Conclusion

Taken together, eight mutations were introduced to pMT1 and improved both activity and product-selectivity by greater than 11,000-fold and 700-fold respectively (Tables 4 and 6). Wild-type pMT1 converted psi-ionone to predominantly trans-α-irone, which is not olfactive. The improved mutant pMT7 converted psi-ionone to more than 90% cis-α-irone, which has the finest, iris-like notes⁵. By further modulating the SAH binding pocket residues, pMT10 with 10-fold improvement of catalytic efficiency was obtained (Table 3), and produced approximately 182 mg/L of cis-α-irone from psi-ionone. By incorporating pMT10 into metabolically engineered Escherichia coli, ˜172 mg/L cis-α-irone was produced from glucose in a fed-batch process. As compared to natural extraction, our current bioprocess is 7,200 to 33,000 fold higher in yield for the same land size and duration (Table 9), paving a way towards sustainable bioproduction of the premium perfume molecule—cis-α-irone.

### Sequences

Protein sequences (the sequences used are codon-optimized with a N-terminal 6×his-tag) and DNA sequences used herein. Other polyhistidine tags may be used as desired and at the C-terminal.

>tr|A0A1Z2L4K2|A0A1Z2L4K2_9ACTN Type
11 methyltransferase OS = Streptomyces albireticuli
OX = 1940 GN = SMD11_3565
PE = 4 SV = 1 (SaMT)-
SEQ ID No. 1
MPQESAQELKVTADEVGDWYDRFGDIYHETLGESVHCGLWFPPDE
PHPTSMDLVDLSSRAQDRYTDYLIETLDPRPGDHVLDIGCGTGRS
ALRLVQQRDARVTGVAISKEQIARADRLANEHGLTDRLTFAYADA
QALPYEDGTFDRAWAVESICHMDRAKALQEAWRVLRPGGDLMVLE
SVLTGELTAEDTAVFQVMLASNLPPTLPEFFGLVGDAGFETLELK
DLSANLAMTMNVMALVCHDRKEEFTERFGAEFMEGVVQGLPKARE
VVARKTRFFLVMLRKPLA
>tr|D5SKC5|D5SKC5_STRC2 Methyltransferase
type 11 OS = Streptomyces clavuligerus
(strain ATCC 27064/DSM 738/JCM 4710/
NBRC 13307/NCIMB 12785/NRRL 3585/
VKM Ac-602) OX = 443255
GN = SCLAV_p0882 PE = 4 SV = 1 (ScMT)-
SEQ ID No. 2
MPQELAGELRVTAAQVGAWYDQFGDIYHQTLGESIHCGLWFPPDE
PHPARVDLVSLSSEAQDRFTDYLIKTLDPHADQHVLDIGCGTGRS
ALRLSQQRGAKVTGVAISKVQIEHANRLAETHDLSDRLVFEHADA
MHLPYEDESFDSAWAIESLCHMDRAKALREAYRVLRPGGDFLLLE
SVLTNPLTEAEATSLDTMLAANTPLWLPEFFELITRAGFETLELK
DLSANLAMTMNVLELVCHDRREEFTRRFGAEFTELLMAGLPEARN
ITARKTRFFMLLLRKPPVPAN
>tr|A0A077K7L1|A0A077K7L1_9ACTN
O-methylransferase
OS = Streptomyces blastmyceticus
OX = 68180 GN = tleD PE = 1 SV = 1 (pMT1)-
SEQ ID No. 3
MPQEARTPQQQVTADEVGDWYDKFGEVYHLTLGESVHCGLWFPPD
APVPQDMELVTMSSQAQDRYTDYLIETLDPKAGQHLLDIGCGTGR
TALKAARQRGIAVTGVAVSKEQIAAANRLAAGHGLTERLTFEVAD
AMRLPYEDESFDCAWAIESLCHMDRAKALGEAWRVLKPGGDLLVL
ESVVTEELTEPETALFETLYAANVPPRLGEFFDIVSGAGFHTLSL
KDLSANLAMTMNVFALGVYSRRAEFTERFGAEFVDGLLAGLGSAQ
ETLIRKTRFFMATLRKPAV
>pMT2-
SEQ ID No. 4
MPQEARTPQQQVTADEVGDWYDKFGEVYHLTLGESVHCGLWFPPD
APVPQDMELVTMSSQAQDRYTDYLIETLDPKAGQHLLDIGCGTGR
TALKAARQRGIAVTGVAVSKEQIAAANRLAAGHGLTERLTFEVAD
AMRLPYEDESFDCAWAIESLCHMDRAKALGEAWRVLKPGGDLLVL
ESVVTEELTEPETALFETLFAANVPPRLGEFFDIVSGAGFHTLSL
KDLSANLAMTMNVFALGVYSRRAEFTERFGAEFVDGLLAGLGSAQ
ETLIRKTRFFMATLRKPAV
>pMT3-
SEQ ID No. 5
MPQEARTPQQQVTADEVGDWYDKFGEVYHLTLGESVHCGLWFPPD
APVPQDMELVTMSSQAQDRYTDYLIETLDPKAGQHLLDIGCGTGR
TALKAARQRGIAVTGVAVSKEQIAAANRLAAGHGLTERLTFEVAD
AMRLPYEDESFDCAWAIESLCHMDRAKALGEAWRVLKPGGDLLVL
EEVVTEELTEPETALFETLFAANVPPRLGEFFDIVSGAGFHTLSL
KDLSANLAMTMNVFALGVYSRRAEFTERFGAEFVDGLLAGLGSAQ
ETLIRKTRFFMATLRKPAV
>pMT4-
SEQ ID No. 6
MPQEARTPQQQVTADEVGDWYDKFGEVYHLTLGESVHCGLWFPPD
APVPQDMELVTMSSQAQDRYTDYLIETLDPKAGQHLLDIGCGTGR
TALKAARQRGIAVTGVAVSKEQIAAANRLAAGHGLTERLTFEVAD
AMRLPYEDESFDCAWAIESLCHMDRAKALGEAWRVLKPGGDLLVL
EEVVTEELTEPETALFETLFAANVPPRLGEFFDIVSGAGFHTLSL
KDLSANLAMTMNVFALGVYSRRAEFTERFGAEFVDGLLAGLGSAQ
ETVIRKTRFFMATLRKPAV
>pMT5-
SEQ ID No. 7
MPQEARTPQQQVTADEVGDWYDKFGEVYHLTLGESVHCGLWFPPD
APVPQDMELVTMSSQAQDRYTDYLIETLDPKAGQHLLDIGCGTGR
TALKAARQRGIAVTGVAVSKEQIAAANRLAAGHGLTERLTFEVAD
AMRLPYEDESFDCAWAIESLCHMDRAKALGEAWRVLKPGGDLLVA
EEVVTEELTEPETALFETLFAANVPPRLGEFFDIVSGAGFHTLSL
KDLSANLAMTMNVFALGVYSRRAEFTERFGAEFVDGLLAGLGSAQ
ETVIRKTRFFMATLRKPAV
>pMT6-
SEQ ID No. 8
MPQEARTPQQQVTADEVGDWYDKFGEVYHLTLGESVHCGLWFPPD
APVPQDMELVTMSSQAQDRYTDYLIETLDPKAGQHLLDIGCGTGR
TALKAARQRGIAVTGVAVSKEQIAAANRLAAGHGLTERLTFEVAD
AMRLPYEDESFDCAWAIESLCHMDRAKALGEAWRVLKPGGDLLVA
EEVVTEELTEPETALFETLFALNVPPRLGEFFDIVSGAGFHTLSL
KDLSANLAMTMNVFALGVYSRRAEFTERFGAEFVDGLLAGLGSAQ
ETVIRKTRFFMATLRKPAV
>pMT7-
SEQ ID No. 9
MPQEARTPQQQVTADEVGDWYDKFGEVYHLTLGESVHCGLWFPPD
APVPQDMELVTMSSQAQDRFTDYLIETLDPKAGQHLLDIGCGTGR
TALKAARQRGIAVTGVAVSKEQIAAANRLAAGHGLTERLTFEVAD
AMRLPYEDESFDCAWAIESLCHMDRAKALGEAWRVLKPGGDLLVA
EEVVTEELTEPETALFETLFALNVPPRLGEFFDIVSGAGFHTLSL
KDLSANLAMTMNVFALGVYSRRAEFTERFGAEFVDGLLAGLGSAQ
ETVIRKTRFFMATLRKPAV
>pMT8-
SEQ ID No. 10
MPQEARTPQQQVTADEVGDWYDKFGEVYHLTLGESVHCGLWFPPD
APVPQDMELVTMSSQAQDRYTDYLIETLDPKAGQHLLDIGCGTGR
TALKAARQRGIAVTGVAVSKEQIAAANRLAAGHGLTERLTFEVAD
AMRLPYEDESFDCAWAIESLPHMDRAKALGEAWRVLKPGGDLLVA
EEVVTEELTEPETALFETLFALNVPPRLGEFFDIVSGAGFHTLSL
KDLSANLAMTMNVFALGVYSRRAEFTERFGAEFVDGLLAGLGSAQ
ETVIRKTRFFMATLRKPAV
>pMT9-
SEQ ID No. 11
MPQEARTPQQQVTADEVGDWYDKFGEVYHLTLGESVHCGLWFPPD
APVPQDMELVTMSSQAQDRYTDYLIETLDPKAGQHLLDIGCGTGR
TALKAARQRGIAVTGVAVSKEQIAAANRLAAGHGLTERLTFEVAD
AMRLPYEDESFDCAWAIESLAHMDRAKALGEAWRVLKPGGDLLVA
EEVVTEELTEPETALFETLFALNVPPRLGEFFDIVSGAGFHTLSL
KDLSANLAMTMNVFALGVYSRRAEFTERFGAEFVDGLLAGLGSAQ
ETVIRKTRFFMATLRKPAV
>pMT10-
SEQ ID No. 12
MPQEARTPQQQVTADEVGDWYDKFGEVYHLTLGESVHCGLWFPPD
APVPQDMELVTMSSQAQDRFTDYLIETLDPKAGQHLLDIGCGTGR
PALKAARQRGIAVTGVAVSKEQIAAANRLAAGHGLTERLTFEVAD
AMRLPYEDESFDCAWAIESLAHMDRAKALGEAWRVLKPGGDLLVA
EEVVTEELTEPETALFETLFALNVPPRLGEFFDIVSGAGFHTLSL
KDLSANLAMTMNVFALGVYSRRAEFTERFGAEFVDGLLAGLGSAQ
ETVIRKTRFFMATLRKPAV
>pMT11-
SEQ ID No. 13
MPQEARTPQQQVTADEVGDWYDKFGEVYHLTLGESVHCGLWFPPD
APVPQDMELVTMSSQAQDRFTDYLIETLDPKAGQHLLDIGCGTGR
PALKAARQRGIAVTGVAVSKEQIAAANRLAAGHGLTERLTFEVAD
AMRLPYEDESFDCAWAIESLDHMDRAKALGEAWRVLKPGGDLLVA
EEVVTEELTEPETALFETLFALNVPPRLGEFFDIVSGAGFHTLSL
KDLSANLAMTMNVFALGVYSRRAEFTERFGAEFVDGLLAGLGSAQ
ETVIRKTRFFMATLRKPAV
>pMT12-
SEQ ID No. 14
MPQEARTPQQQVTADEVGDWYDKFGEVYHLTLGESVHCGLWFPPD
APVPQDMELVTMSSQAQDRFTDYLIETLDPKAGQHLLDIGCGTGR
PALKAARQRGIAVTGVAVSKEQIAAANRLAAGHGLTERLTFEVAD
AMRLPYEDESFDCAWAIESLDHMDRAKALGEAWRVLKPGGDLLVA
EEVVTEELTEPETALFETLFALNVPPRLGEFFDIVSGAGFHTLSL
KDLSADLAMTMNVFALGVYSRRAEFTERFGAEFVDGLLAGLGSAQ
ETVIRKTRFFMATLRKPAV
>pMT13-
SEQ ID No. 15
MPQEARTPQQQVTADEVGDWYDKFGEVYHLTLGESVHCGLWFPPD
APVPQDMELVTMSSQAQDRFTDYLIETLDPKAGQHLLDIGCGTGR
PALKAARQRGIAVTGVAVSKEQIAAANRLAAGHGLTERLTFEVAD
AMRLPYEDESFDCAWAIESLDHMDRAKALGEAWRVLKPGGDLLVA
EEVVTEELTEPETALFETLFALNVPPRLGEFFDIVSGAGFHTLSL
KDLSADLAMTMNVFALGVAARRAEFTERFGAEFVDGLLAGLPSAQ
ETVIRKTRFFMATLRKPAV
>pMT14-
SEQ ID No. 16
MPQEARTPQQQVTADEVGDWYDKFGEVYHLTLGESVHCGLWFPPD
APVPQDMELVTMSSQAQDRFTDYLIETLDPKAGQHLLDIGCGTGR
PALKAARQRGIAVTGVAVSKEQIAAANRLAAGHGLTERLTFEVAD
AMRLPYEDESFDCAWAIESLDHMDRAKALGEAWRVLKPGGDLLVA
EEVVTEELTEPETALFRTLFALNVPPRLGEFFDIVSGAGFHTLSL
KDLSADLAMTMNVFALGVAARRAEFTERFGAEFVDGLLAGLPSAQ
ETVIRKTRFFMATLRKPAV
Codon optimized sequences used to
produce the modified enzymes above
>tr|A0A1Z2L4K2|A0A1Z2L4K2_9ACTN Type 11
methyltransferase OS = Streptomyces albireticuli
OX = 1940 GN = SMD11_3565 PE = 4 SV = 1 (SaMT)-
SEQ ID No. 17
atgcatcatcatcaccatcacccgcaggagtctgcgcaggaactg
aaagttaccgcggacgaagttggggattggtacgatcgtttcggt
gacatctaccacgaaaccctcggagaaagcgtgcactgcggcctg
tggtttccgccggatgaaccacatccgactagcatggatctggta
gacctgtcttctcgcgcccaagatcgttacaccgattacctgatc
gaaaccctggatccgcgcccgggcgaccacgtgctggacattggt
tgtggcaccggtcgttctgcgctccgtctggtacaacaacgcgac
gcgcgtgttaccggtgtagctatttctaaagagcagatcgcccgt
gcggaccgcctggcgaatgaacacggtctgaccgaccgcctgacc
ttcgcatacgcagatgctcaagctctgccgtacgaagatggtacc
tttgatcgcgcttgggcggttgagtccatctgccacatggaccgt
gcgaaagctctgcaggaagcgtggcgtgttttacgccctggtgga
gatctgatggttctggaatctgtgctgaccggtgaactgaccgcg
gaagatacggctgtgttccaggtaatgctggcgtctaacctgccg
ccgactctgccggaattcttcggcctggttggtgacgcaggcttt
gaaaccctggaactgaaagacttatctgcgaacctggctatgacc
atgaacgtgatggccctggtttgccatgaccgcaaagaagaattc
accgaacgttttggcgcggaattcatggagggtgtagtacagggt
ctcccgaaggcgcgcgaagttgttgcacgtaaaacccgttttttc
ctcgtgatgctgcgcaaaccgctggcttaa
>tr|D5SKC5|D5SKC5_STRC2 Methyltransferase
type 11 OS = Streptomyces clavuligerus
(strain ATCC 27064/DSM 738/JCM 4710/
NBRC 13307/NCIMB 12785/NRRL 3585/
VKM Ac-602) OX = 443255 GN = SCLAV_p0882
PE = 4 SV = 1 (ScMT)-
SEQ ID No. 18
atgcatcatcatcaccatcacccacaggaactgggggtgaactgc
gtgtgaccgctgcccaggtaggcgcgtggtatgatcagttcggtg
acatctaccaccaaaccttaggtgagtccatccattgcggtctgt
ggttcccaccggacgaaccgcatccagctcgtgttgatctggttt
ctctgtcgagcgaagcgcaggatcgtttcaccgactaccttatta
aaacgctggacccgcacgcagaccagcacgttctggatatcggtt
gtggcactggccgctctgcactgcgtctgtcgcaacagcgtggtg
caaaggttaccggcgtagcaatttccaaagtgcagatcgaacatg
ccaatcgcctggcggaaacccacgacttgagcgatcgtctggtct
tcgaacacgcagatgctatgcatctgccgtatgaagatgaatctt
tcgattccgcttgggctatcgaatccctctgccacatggaccgcg
cgaaagcgctgcgcgaagcttaccgtgtactgcgcccgggtggcg
atttcctgctgctggagagcgttctgaccaacccgctgaccgaag
cagaagccaccagcttagatactatgctggcggcaaataccccgc
tgtggctgccagaattctttgaactgatcacccgtgctggttttg
aaactctggaactgaaagacctgagcgcgaacctggccatgacca
tgaacgttctggaactggtttgccatgatcgtcgcgaagaattta
cccgtcgttttggtgctgagtttaccgaactgttgatggctggtc
tgccagaggcgcgtaacatcaccgcgcgcaagacgcgtttcttta
tgctgctgctgcgtaagccgccggtgccggcgaactaa
>tr|A0A077K7L1|A0A077K7L1_9ACTN
O-methylransferase
OS = Streptomyces blastmyceticus
OX = 68180 GN-tleD
PE = 1 SV = 1 (pMT1)-
SEQ ID No. 19
atgcatcatcatcaccatcacgtcccgcaggaagcccgtaccccg
cagcagcaggttaccgccgatgaagtcggcgattggtacgataaa
ttcggcgaagtgtaccatctgactctgggtgaaagcgtgcattgc
ggtctgtggttcccgccggacgccccggttccgcaggacatggag
ctggttaccatgtcctctcaggcgcaggatcgttacacggattat
ctgattgaaaccctggatccgaaagcgggtcagcatctgttagac
atcggctgtggtaccggtcgcaccgctctgaaagccgcacgccag
cgcggtatcgcggtgaccggtgtagcagttagtaaagaacagatt
gctgcagcgaaccgcctggcggcaggtcacggtctgactgagcgt
ctgaccttcgaagtagccgacgctatgcgtctgccgtacgaagac
gaatcgttcgactgtgcttgggcgatcgagtcactgtgccacatg
gatcgtgcaaaggctcttggtgaagcttggcgtgtcttgaaaccg
ggtggtgacctgctggtactggaatccgtcgtaactgaagaactg
actgaaccggaaaccgcactgttcgaaacgctgtacgccgcgaat
gttccgccgcgtctgggtgaattctttgatatcgtatctggtgcg
ggtttccacaccctgagcttaaaagacctgtccgcaaacctggcc
atgactatgaacgttttcgcactgggtgtgtattctcgtcgtgcc
gaatttaccgaacgcttcggcgcggaattcgttgacggcctgctg
gccggtctgggctcggcgcaggaaaccctcattcgcaaaacccgt
ttctttatggctactctgcgcaagccggcggtctaa

SEQ ID Numbers 4 to 16 and 20 to 100 are mutations introduced to SEQ ID No. 3 (pMT1) and the codon optimized sequence may be derived from SEQ ID No. 19. SEQ ID No. 101 to 114 set out example of sequences that may be used in the plasmid for in vivo production of cis-α-irone in the following order respectively: HmgS, AtoB, tHmgR, MevK, PMK, PMD, Idi, crtE, crtB, Crtl, ispA, TOfCCD1m, MetK, and mtn.

### REFERENCES REFERRED TO AND INCORPORATED HEREIN IN ITS ENTIRETY

(1) Shukal, S.; Chen, X.; Zhang, C. Systematic Engineering for High-Yield Production of Viridiflorol and Amorphadiene in Auxotrophic Escherichia Coli. Metab. Eng. 2019, 55, 170-178. https://doi.org/10.1016/j.ymben.2019.07.007.
(2) Gil, G.; Petit, J. L.; Seris, J.-L. Process for Obtaining Irone by Enzymatic Route. U.S. Pat. No. 5,100,790A, Mar. 31, 1992.
(3) Brenna, E.; Fuganti, C.; Fronza, G.; Malpezzi, L.; Righetti, A.; Serra, S. Enzyme-Mediated Preparation of (+)- and (−)-Cis-α-Irone and (+)- and (−)-Trans-α-Irone. Helv. Chim. Acta 1999, 82 (12), 2246-2259. https://doi.org/10.1002/(SICI)1522-2675(19991215)82:12<2246::AID-HLCA2246>3.0.CO; 2-3.
(4) König, W. A.; Hochmuth, D. H. Enantioselective Gas Chromatography in Flavor and Fragrance Analysis: Strategies for the Identification of Known and Unknown Plant Volatiles. J. Chromatogr. Sci. 2004, 42 (8), 423-439. https://doi.org/10.1093/chromsci/42.8.423.
(5) Brenna, E.; Fuganti, C.; Serra, S. Applications of Biocatalysis in Fragrance Chemistry: The Enantiomers of α-, β-, and γ-Irones. Chem. Soc. Rev. 2008, 37 (11), 2443-2451. https://doi.org/10.1039/B801557K.
(6) Marner, F.-J.; Gladtke, D.; Jaenicke, L. Studies on the Biosynthesis of Iridals and Cycloiridals. Helv. Chim. Acta 1988, 71 (5), 1331-1338. https://doi.org/10.1002/hlca. 19880710544.
(7) Chen, X.; Shukal, S.; Zhang, C. Integrating Enzyme and Metabolic Engineering Tools for Enhanced α-Ionone Production. J. Agric. Food Chem. 2019. https://doi.org/10.1021/acs.jafc.9b00860.
(8) Zhang, C.; Chen, X.; Lindley, N. D.; Too, H.-P. A “Plug-n-Play” Modular Metabolic System for the Production of Apocarotenoids. Biotechnol. Bioeng. 2018, 115 (1), 174-183. https://doi.org/10.1002/bit.26462.
(9) Awakawa, T.; Zhang, L.; Wakimoto, T.; Hoshino, S.; Mori, T.; Ito, T.; Ishikawa, J.; Tanner, M. E.; Abe, I. A Methyltransferase Initiates Terpene Cyclization in Teleocidin B Biosynthesis. J. Am. Chem. Soc. 2014, 136 (28), 9910-9913. https://doi.org/10.1021/ja505224r.
(10) Yu, F.; Li, M.; Xu, C.; Sun, B.; Zhou, H.; Wang, Z.; Xu, Q.; Xie, M.; Zuo, G.; Huang, P.; Guo, H.; Wang, Q.; He, J. Crystal Structure and Enantioselectivity of Terpene Cyclization in SAM-Dependent Methyltransferase TleD. Biochem. J. 2016, 473 (23), 4385-4397. https://doi.org/10.1042/BCJ20160695.
(11) NCBI Resource Coordinators. Database Resources of the National Center for Biotechnology Information. Nucleic Acids Res. 2018, 46 (D1), D8-D13. https://doi.org/10.1093/nar/gkx1095.
(12) Zhang, C.; Sultan, S. A.; T, R.; Chen, X. Biotechnological Applications of S-Adenosyl-Methionine-Dependent Methyltransferases for Natural Products Biosynthesis and Diversification. Bioresour. Bioprocess. 2021, 8 (1), 72. https://doi.org/10.1186/s40643-021-00425-y.
(13) Hermann, J. C.; Marti-Arbona, R.; Fedorov, A. A.; Fedorov, E.; Almo, S. C.; Shoichet, B. K.; Raushel, F. M. Structure-Based Activity Prediction for an Enzyme of Unknown Function. Nature 2007, 448 (7155), 775-779. https://doi.org/10.1038/nature05981.
(14) Wang, J. X.; Lee, E. R.; Morales, D. R.; Lim, J.; Breaker, R. R. Riboswitches That Sense S-Adenosylhomocysteine and Activate Genes Involved in Coenzyme Recycling. Mol. Cell 2008, 29 (6), 691-702. https://doi.org/10.1016/j.molcel.2008.01.012.
(15) Zhang, C.; Seow, V. Y.; Chen, X.; Too, H.-P. Multidimensional Heuristic Process for High-Yield Production of Astaxanthin and Fragrance Molecules in Escherichia Coli. Nat. Commun. 2018, 9 (1), 1-12. https://doi.org/10.1038/s41467-018-04211-x.
(16) Cress, B. F.; Leitz, Q. D.; Kim, D. C.; Amore, T. D.; Suzuki, J. Y.; Linhardt, R. J.; Koffas, M. A. G. CRISPRi-Mediated Metabolic Engineering of E. Coli for O-Methylated Anthocyanin Production. Microb. Cell Factories 2017, 16 (1), 1-14. https://doi.org/10.1186/s12934-016-0623-3.
(17) Kunjapur, A. M.; Hyun, J. C.; Prather, K. L. J. Deregulation of S-Adenosylmethionine Biosynthesis and Regeneration Improves Methylation in the E. Coli de Novo Vanillin Biosynthesis Pathway. Microb. Cell Factories 2016, 15 (1), 1-17. https://doi.org/10.1186/s12934-016-0459-x.
(18) Shukal, S.; Lim, X. H.; Zhang, C.; Chen, X. Metabolic Engineering of Escherichia Coli BL21 Strain Using Simplified CRISPR-Cas9 and Asymmetric Homolog Arms Recombineering. Sep. 28, 2021. https://doi.org/10.21203/rs.3.rs-889623/v1.
(19) Studier, F. W. Protein Production by Auto-Induction in High-Density Shaking Cultures. Protein Expr. Purif. 2005, 41 (1), 207-234. https://doi.org/10.1016/j.pep.2005.01.016.
(20) Hanwell, M. D.; Curtis, D. E.; Lonie, D. C.; Vandermeersch, T.; Zurek, E.; Hutchison, G. R. Avogadro: An Advanced Semantic Chemical Editor, Visualization, and Analysis Platform. J. Cheminformatics 2012, 4 (1), 17. https://doi.org/10.1186/1758-2946-4-17.
(21) Webb, B.; Sali, A. Comparative Protein Structure Modeling Using MODELLER. Curr. Protoc. Bioinforma. 2016, 54, 5.6.1-5.6.37. https://doi.org/10.1002/cpbi.3.
(22) Maier, J. A.; Martinez, C.; Kasavajhala, K.; Wickstrom, L.; Hauser, K. E.; Simmerling, C. Ff14SB: Improving the Accuracy of Protein Side Chain and Backbone Parameters from Ff99SB. J. Chem. Theory Comput. 2015, 11 (8), 3696-3713. https://doi.org/10.1021/acs.jctc.5b00255.
(23) Development and testing of a general amber force field—Wang—2004—Journal of Computational Chemistry Wiley Online Library https://onlinelibrary.wiley.com/doi/10.1002/jcc.20035 (accessed 2022-01-16).
(24) Salomon-Ferrer, R.; Gotz, A. W.; Poole, D.; Le Grand, S.; Walker, R. C. Routine Microsecond Molecular Dynamics Simulations with AMBER on GPUs. 2. Explicit Solvent Particle Mesh Ewald. J. Chem. Theory Comput. 2013, 9 (9), 3878-3888. https://doi.org/10.1021/ct400314y.
(25) Fast, efficient generation of high-quality atomic charges. AM1-BCC model: Il. Parameterization and validation—Jakalian—2002—Journal of Computational Chemistry Wiley Online Library https://onlinelibrary.wiley.com/doi/abs/10.1002/jcc.10128 (accessed 2022 Jan. 16).
(26) Voss, M.; Xiang, C.; Esque, J.; Nobili, A.; Menke, M. J.; André, I.; Höhne, M.; Bornscheuer, U. T. Creation of (R)-Amine Transaminase Activity within an α-Amino Acid Transaminase Scaffold. ACS Chem. Biol. 2020, 15 (2), 416-424. https://doi.org/10.1021/acschembio.9b00888.
(27) MMPBSA.py: An Efficient Program for End-State Free Energy Calculations|Journal of Chemical Theory and Computation https://pubs.acs.org/doi/10.1021/ct300418h (accessed 2022 Jan. 16).
(28) Gerlt, J. A.; Bouvier, J. T.; Davidson, D. B.; Imker, H. J.; Sadkhin, B.; Slater, D. R.; Whalen, K. L. Enzyme Function Initiative-Enzyme Similarity Tool (EFI-EST): A Web Tool for Generating Protein Sequence Similarity Networks. Biochim. Biophys. Acta BBA—Proteins Proteomics 2015, 1854 (8), 1019-1037. https://doi.org/10.1016/j.bbapap.2015.04.015.
(29) Sumbalova, L., Stourac, J., Martinek, T., Bednar, D. & Damborsky, J. HotSpot Wizard 3.0: web server for automated design of mutations and smart libraries based on sequence input information. Nucleic Acids Res 46, W356-W362 (2018).

1. A method of producing cis-α-irone, the method comprises providing an enzyme capable of converting psi-ionone to cis-α-irone; and contacting the enzyme and psi-ionone under suitable conditions to produce cis-α-irone.

2. The method according to claim 1 wherein the enzyme is selected from the group consisting of a modified enzyme, SEQ ID No. 1, and SEQ ID No. 3, the modified enzyme comprising a first substitution of base SEQ ID No. 3 at a position selected from the group consisting of position 200, position 180, position 160, and position 236,
wherein if the first substitution is at position 200, the first substitution is selected from the group consisting of phenylalanine, isoleucine, leucine, valine, and tryptophan,
wherein if the first substitution is at position 180, the first substitution is selected from the group consisting of alanine, cysteine, glutamic acid, isoleucine, methionine, and valine,
wherein if the first substitution is at position 160, the first substitution is selected from the group consisting of alanine, cysteine, histidine, isoleucine, leucine, methionine, asparagine, glutamine, threonine, valine, and tyrosine,
wherein if the first substitution is at position 236, the first substitution is selected from the group consisting of cysteine, glutamic acid, histidine, isoleucine, leucine, asparagine, serine, threonine, and valine.

3. The method according to claim 2, the modified enzyme comprising a second substitution selected from the group consisting of position 182 and position 180, wherein if the second substitution is at position 182, the second substitution is selected from the group consisting of glutamic acid, threonine, asparagine and glutamine, wherein the second substitution may be selected at position 180 if only the first substitution is not at position 180, and if the second substitution is at position 180, the second substitution is selected from the group consisting of alanine, cysteine.

4. The method according to any of claims 2 to 3, the modified enzyme comprising a third substitution at position 273, wherein the third substitution is selected from the group consisting of valine, isoleucine, and lysine.

5. The method according to any one of claims 2 to 4, the modified enzyme comprising a fourth substitution at position 180, wherein the first substitution is not at position 180 and the second substitution if present is not at position 180, and the fourth substitution is alanine or cysteine.

6. The method according to any one of claims 2 to 5, the modified enzyme comprising a fifth substitution at position 202 to a bulkier amino acid.

7. The method according to claim 6 wherein the fifth substitution is selected from the group consisting of leucine, valine, and phenylalanine.

8. The method according to any one of claims 2 to 7, the modified enzyme comprising a sixth substitution at position 65, wherein the sixth substitution is selected from the group consisting of phenylalanine, leucine, and methionine.

9. The method according to any one of claims 2 to 8, the modified enzyme comprising a seventh substitution at position 156, wherein the seventh substitution is selected from the group consisting of aspartic acid, alanine, proline, glycine, and serine.

10. The method according to any one of claims 2 to 9, the modified enzyme comprising an eighth substitution at position 91, wherein the eighth substitution is proline.

11. The method according to any one of claims 2 to 10, the modified enzyme comprising a ninth substitution at position 231, wherein the ninth substitution is aspartic acid.

12. The method according to any one of claims 2 to 11, the modified enzyme comprising a tenth substitution selected from the group consisting of alanine at position 244, alanine at position 245, and proline at position 267.

13. The method according to any one of claims 2 to 12, the modified enzyme comprising an eleventh substitution at position 197, wherein the eleventh substitution is arginine.

14. The method according to any one of claims 2 to 13, the modified enzyme comprising a twelfth substitution at position 60, wherein the twelfth substitution is selected from the group consisting of valine, lysine, and arginine.

15. The method according to any one of claims 2 to 13, the modified enzyme comprising a thirteenth substitution selected from the group consisting of:
(i) the thirteenth substitution is at position 11 and selected from histidine, leucine, proline, methionine, valine, and tryptophan;
(ii) the thirteenth substitution is at position 12 and selected from lysine, alanine glycine, and arginine;
(iii) the thirteenth substitution is at position 13 and selected from leucine, methionine, glutamine, alanine, and glycine;
(iv) the thirteenth substitution is at position 14 and selected from methionine, arginine, glycine, proline, leucine, and threonine;
(v) the thirteenth substitution is at position 94 and selected from arginine, and valine;
(vi) the thirteenth substitution is at position 95 and selected from isoleucine, cysteine, valine, and leucine;
(vii) the thirteenth substitution is at position 107 and selected from serine and glycine;
(viii) the thirteenth substitution is at position 123 and selected from asparagine, glutamine, and serine;
(ix) the thirteenth substitution is at position 126 and selected from serine and glutamic acid;
(x) the thirteenth substitution is at position 127 and selected from glycine and threonine;
(xi) the thirteenth substitution is at position 129 and selected from cysteine, lysine, and valine;
(xii) the thirteenth substitution is at position 137 and selected from glycine, alanine, aspartic acid, histidine, asparagine, serine, and threonine;
(xiii) the thirteenth substitution is at position 159 and is leucine;
(xiv) the thirteenth substitution is at position 176 and selected from leucine, valine, lysine, arginine and tyrosine, and optionally a fourteenth substitution of valine at position 248;
(xv) the thirteenth substitution is at position 185 and selected from valine and leucine;
(xvi) the thirteenth substitution is at position 190 and selected from glutamine, alanine and proline, and optionally a fifteenth substitution of serine at position 122;
(xvii) the thirteenth substitution is at position 191 and selected from serine, valine, leucine, isoleucine, tyrosine, and lysine;
(xviii) the thirteenth substitution is at position 192 and selected from serine;
(xix) the thirteenth substitution is at position 195 and selected from isoleucine and valine;
(xx) the thirteenth substitution is at position 212 and is leucine;
(xxi) the thirteenth substitution is at position 268 and selected from lysine, glutamine, histidine, arginine, and leucine;
(xxii) the thirteenth substitution is at position 269 and selected from leucine, glycine, arginine, and tryptophan; and
(xxiii) the thirteenth substitution is at position 272 and is alanine.

16. The method according to claim 2 wherein the modified enzyme comprises a sequence selected from the group consisting of SEQ ID No. 15, SEQ ID No. 14, SEQ ID No. 13, SEQ ID No. 16, SEQ ID No. 12, SEQ ID No. 11, SEQ ID No. 10, SEQ ID No. 9, SEQ ID No. 8, SEQ ID No. 7, SEQ ID No. 6, SEQ ID No. 5, SEQ ID No. 4, and SEQ ID No. 20 to 100.

17. The method according to any one of claims 1 to 16, the modified enzyme comprising a polyhistidine-tag.

18. The method according to any one of claims 1 to 17 wherein contacting the modified enzyme and psi-ionone is done in the presence of an auxiliary enzyme that removes or recycles SAH.

19. The method according to claim 18 wherein the auxiliary enzyme is selected from the group consisting of S-adenosylmethionine synthase (MetK), adenosylhomocysteinase (SAH1), 5′-methylthioadenosine/S-adenosylhomocysteine nucleosidase (mtn), 5-methylthioadenosine/S-adenosylhomocysteine deaminase (mtaD), and halide methyl transferase (HMT).

20. The method according to any one of claims 1 to 19 wherein psi-ionone has a maximum concentration of 5 mM.

21. The method according to any one of claims 1 to 20 wherein the modified enzyme is provided as a lysate of a cell expressing the modified enzyme or a host cell comprising a plurality of nucleic acid sequences to encode at least one host cell enzyme and a first nucleic acid sequence encoding the modified enzyme, wherein the plurality of enzymes are produced by the host cell to assist in converting glucose or glycerol to psi-ionone.

22. The method according to claim 21 wherein the host cell enzyme includes at least one of the following: HMG-COA synthase (HmgS), Acetoacetyl-CoA thiolase (AtoB), truncated HMG-COA reductase (tHmgR) or HMG-COA reductase, mevalonate kinase (MevK), phosphomevalonate kinase (PMK), mevalonate pyrophosphate decarboxylase (PMD), IPP isomerase (Idi), GGPP synthase (CrtE), phytoene synthase (CrtB), phytoene desaturase (Crtl), FPP synthase (IspA), and modified OfCCD1 enzyme or OfCCD1 enzyme.

23. The method according to any one of claims 21 to 22, wherein at least one of the following conditions are fulfilled:
(i) the host cell further comprises a second nucleic acid sequence to encode SAM cycle enzymes;
(ii) a third nucleic acid sequence encoding a transcription regulator metJ is absent in the host cell;
(iii) the host cell further comprises at least one T7 promoter sequence;
(iv) the host cell is Escherichia coli.

24. The method according to any one of claims 21 to 23 wherein the modified enzyme is provided by the host cell and the suitable conditions include a dissolved oxygen content of 30% or less, preferably 1% to 15%, more preferably 2% to 10%.

25. The method according to any one of claims 1 to 24 wherein at least 0.1 μg/L of cis-α-irone is produced or a minimum cis-to-trans α-irone ratio of 2 to 5.

26. A modified enzyme comprising a first substitution of base SEQ ID No. 3 at a position selected from the group consisting of position 200, position 180, position 160, and position 236,
wherein if the first substitution is at position 200, the first substitution is selected from the group consisting of phenylalanine, isoleucine, leucine, valine, and tryptophan,
wherein if the first substitution is at position 180, the first substitution is selected from the group consisting of alanine, cysteine, glutamic acid, isoleucine, methionine, and valine,
wherein if the first substitution is at position 160, the first substitution is selected from the group consisting of alanine, cysteine, histidine, isoleucine, leucine, methionine, asparagine, glutamine, threonine, valine, and tyrosine,
wherein if the first substitution at position 236 is selected, the first substitution is selected from the group consisting of cysteine, glutamic acid, histidine, isoleucine, leucine, asparagine, serine, threonine, and valine.

27. The modified enzyme according to claim 26 comprising a second substitution selected from the group consisting of position 182 and position 180, wherein if the second substitution is at position 182, the second substitution is selected from the group consisting of glutamic acid, threonine, asparagine and glutamine, wherein the second substitution may be selected at position 180 only if the first substitution is not at position 180, and if the second substitution is at position 180, the second substitution is selected from the group consisting of alanine, cysteine.

28. The modified enzyme according to any one of claims 26 to 27 comprising a third substitution at position 273, wherein the third substitution is selected from the group consisting of valine, isoleucine, and lysine.

29. The modified enzyme according to any one of claims 26 to 28 comprising a fourth substitution at position 180, wherein the first substitution is not at position 180 and the second substitution if present is not at position 180, and the fourth substitution is alanine or cysteine.

30. The modified enzyme according to any one of claims 26 to 29 comprising a fifth substitution at position 202 to a bulkier amino acid.

31. The modified enzyme according to claim 30 wherein the fifth substitution is selected from the group consisting of leucine, valine, and phenylalanine.

32. The modified enzyme according to any one of claims 26 to 31 comprising a sixth substitution at position 65, wherein the sixth substitution is selected from the group consisting of phenylalanine, leucine, and methionine.

33. The modified enzyme according to any one of claims 26 to 32 comprising a seventh substitution at position 156, wherein the seventh substitution is selected from the group consisting of aspartic acid, alanine, proline, glycine, and serine.

34. The modified enzyme according to any one of claims 26 to 33 comprising an eighth substitution at position 91, wherein the eighth substitution is proline.

35. The modified enzyme according to any one of claims 26 to 34, the modified enzyme comprising a ninth substitution at position 231, wherein the ninth substitution is aspartic acid.

36. The modified enzyme according to any one of claims 26 to 35, the modified enzyme comprising a tenth substitution selected from the group consisting of alanine at position 244, alanine at position 245, and proline at position 267.

37. The modified enzyme according to any one of claims 26 to 36, the modified enzyme comprising an eleventh substitution at position 197, wherein the eleventh substitution is arginine.

38. The modified enzyme according to any one of claims 26 to 37, the modified enzyme comprising a twelfth substitution at position 60, wherein the twelfth substitution is selected from the group consisting of valine, lysine, and arginine.

39. The modified enzyme according to any one of claims 26 to 38, the modified enzyme comprising a thirteenth substitution selected from the group consisting of:
(i) the thirteenth substitution is at position 11 and selected from histidine, leucine, proline, methionine, valine, and tryptophan;
(ii) the thirteenth substitution is at position 12 and selected from lysine, alanine, glycine, and arginine;
(iii) the thirteenth substitution is at position 13 and selected from leucine, methionine, glutamine, alanine, and glycine;
(iv) the thirteenth substitution is at position 14 and selected from methionine, arginine, glycine, proline, leucine, and threonine;
(v) the thirteenth substitution is at position 94 and selected from arginine, and valine;
(vi) the thirteenth substitution is at position 95 and selected from isoleucine, cysteine, valine, and leucine;
(vii) the thirteenth substitution is at position 107 and selected from serine and glycine;
(viii) the thirteenth substitution is at position 123 and selected from asparagine, glutamine, and serine;
(ix) the thirteenth substitution is at position 126 and selected from serine and glutamic acid;
(x) the thirteenth substitution is at position 127 and selected from glycine and threonine;
(xi) the thirteenth substitution is at position 129 and selected from cysteine, lysine, and valine;
(xii) the thirteenth substitution is at position 137 and selected from glycine, alanine, aspartic acid, histidine, asparagine, serine, and threonine;
(xiii) the thirteenth substitution is at position 159 and is leucine;
(xiv) the thirteenth substitution is at position 176 and selected from leucine, valine, lysine, arginine, and tyrosine, and optionally a fourteenth substitution of valine at position 248;
(xv) the thirteenth substitution is at position 185 and selected from valine and leucine;
(xvi) the thirteenth substitution is at position 190 and selected from glutamine, alanine and proline, and optionally a fifteenth substitution of serine at position 122;
(xvii) the thirteenth substitution is at position 191 and selected from serine, valine, leucine, isoleucine, tyrosine, and lysine;
(xviii) the thirteenth substitution is at position 192 and selected from serine;
(xix) the thirteenth substitution is at position 195 and selected from isoleucine, and valine;
(xx) the thirteenth substitution is at position 212 and is leucine;
(xxi) the thirteenth substitution is at position 268 and selected from lysine, glutamine, histidine, arginine, and leucine;
(xxii) the thirteenth substitution is at position 269 and selected from leucine, glycine, arginine, and tryptophan; and
(xxiii) the thirteenth substitution is at position 272 and is alanine.

40. The modified enzyme according to claim 26 wherein the modified enzyme comprises a sequence selected from the group consisting of SEQ ID No. 15, SEQ ID No. 14, SEQ ID No. 13, SEQ ID No. 16, SEQ ID No. 12, SEQ ID No. 11, SEQ ID No. 10, SEQ ID No. 9, SEQ ID No. 8, SEQ ID No. 7, SEQ ID No. 6, SEQ ID No. 5, SEQ ID No. 4, and SEQ ID No. 20 to 100.

41. The modified enzyme according to any of claims 26 to 40 further comprising a polyhistidine-tag.

42. A host cell comprising a plurality of nucleic acid sequences to encode enzymes to allow the host cell to convert glucose or glycerol to psi-ionone and a first nucleic acid sequence encoding the modified enzyme according to any of claims 26 to 41.

43. The host cell according to claim 42 wherein the enzymes include at least one of the following: HMG-COA synthase (HmgS), Acetoacetyl-CoA thiolase (AtoB), truncated HMG-CoA reductase (tHmgR) or HMG-COA reductase, mevalonate kinase (MevK), phosphomevalonate kinase (PMK), mevalonate pyrophosphate decarboxylase (PMD), IPP isomerase (Idi), GGPP synthase (CrtE), phytoene synthase (CrtB), phytoene desaturase (Crtl), FPP synthase (IspA), and modified OfCCD1 enzyme or OfCCD1 enzyme.

44. The host cell according to any of claims 42 to 43 wherein at least one of the following conditions is fulfilled:
(i) further comprising a second nucleic acid sequence to encode SAM cycle enzymes;
(ii) a third nucleic acid sequence encoding a transcription regulator metJ is absent;
(iii) comprising at least one T7 promoter sequence;
(iv) the host cell is Escherichia coli

45. A method of methylating a hydroxyl group, the method comprising providing an enzyme selected from the group consisting of the modified enzyme according to any one of claims 26 to 41 (preferably SEQ ID No. 8), SEQ ID No. 1, and SEQ ID No. 3, or the host cell according to any one of claims 42 to 44; and contacting the enzyme or the host cell and a hydroxyl group under suitable conditions to methylate the hydroxyl group.

46. The method according to claim 45 wherein the hydroxyl group is an allylic hydroxyl group.
